SEQUENCE LISTING (11 sequences)

SEQ ID NO: 1 - DNA, 1509 bp, Unknown
  Akkermansia species GGCC_0220 16S rRNA gene
aaaggaggtgatccagccgcaggttcccctacggctaccttgttacgacttcatcccagttaccagtctcaccttaggaccctgcctccttgcggttggcttcagatacttcgggtgcgaccggcttccatgatgtgacgggcggtgtgtacaagacccgggaacgtattcacggcgccgtagctgatgcgccattactagcgattccggcttcgtgtaggcgggttgcagcctacagtccgaactgggcccagtttttaggatttcctccgcctcgcggcttcggccccctctgtactgggcattgtagtacgtgtgcagccctgggcataagggccatactgacctgacgtcgtccccaccttcctcccagttgatctgggcagtctcgccagagtccccacctttacgtgctggtaactggcaacaggggttgcgctcgttgctggacttaaccaaacatctcacgacacgagctgacgacggccatgcagcacctgtgtaacgcctccgaagagtcgcatgctttcacatgttgttcattacatgtcaagcccaggtaaggttcttcgcgttgcatcgaattaagccacatactccaccgcttgtgcgggtccccgtcaatttctttgagttttaatcttgcgaccgtactccccaggcggcacgcttaacgcgttagctccggcacgcagggggtcgattccccgcacaccaagcgtgcaccgtttactgccaggactacaggggtatctaatccctttcgctcccctggccttcgtgcctcagcgtcagttaatgtccaggaacccgccttcgccacgagtgttcctctcgatatctacgcatttcactgctacaccgagaattccggttccccctccattactctagtctcgcagtatcatgtgcagtccgggggttgagcccccgcctttcacacacgacttacgaaaccgcctacgcacgctttacgcccagtgattccgaacaacgcttgagacctctgtattaccgcggctgctggcacagagttagccgtctcttcctcttgtggtactatctttttaatttgctcccacatgacaggggtttacaatccgaagaccttcattcccccacgcggcgtcgcaccatcagggtttcccccattgtgaatgattctcgactgctgccacccgtaggtgtctggaccgtgtctcagttccagtgtggccggacatcctctcagaccggctacccgtcatcgccttggtgagccgttacctcaccaactaactaataggacgcgagcccatccccaagcgcattgctgctttaatcttgcgattttatgcggtattaatcccagtttcccggggctatcccactcttgggggcaggttactcacgtgttactcacccgtgcgccactagagaattattagcaagctagcaattctctcgttcgacttgcatgtcttatccacgccgccagcgttcgttctgagccagaatcaaactctccat

SEQ ID NO: 2 - DNA, 1523 bp, Unknown
  Bacteroides uniformis GGCC_0301 16S rRNA gene
aaggaggtgttccagccgcaccttccggtacggctaccttgttacgacttagccccaatcaccagttttaccctagggcgctccttgcggttacgcacttcaggtacccccggctttcatggcttgacgggcggtgtgtacaaggcccgggaacgtattcaccgcgccgtggctgatgcgcgattactagcgaatccagcttcatggagtcgggttgcagactccaatccgaactgagagaggctttcgggattagcatcacgtcgccgtgtagctgccttctgtaccccccattgtaacacgtgtgtagccccggacgtaagggccgtgctgatttgacgtcatccccaccttcctcacatcttacgacggcagtctcgacagagtccccagcataacctgatggtaactatcgataagggttgcgctcgttatggcacttaagccgacacctcacggcacgagctgacgacaaccatgcagcaccttcacaactgccttgcggctgacatgtctccacatcattcagttgcaattcaagcccgggtaaggttcctcgcgtatcatcgaattaaaccacatgttcctccgcttgtgcgggcccccgtcaattcctttgagtttcaccgttgccggcgtactccccaggtggaatacttaacgctttcgcttggccgcttactgtatatcgcaaacagcgagtattcatcgtttactgtgtggactaccagggtatctaatcctgtttgatacccacactttcgagcatcagcgtcagttacagtccagcaggctgccttcgcaatcggagttcttcgtgatatctaagcatttcaccgctacaccacgaattccgcctgcctctactgtactcaagacacccagtatcaactgcaattttacggttgagccgcaaactttcacaactgacttaagcgtccgcctacgctccctttaaacccaataaatccggataacgctcggatcctccgtattaccgcggctgctggcacggagttagccgatccttattcatacggtacatacaaaaaggcacacgtgcctcactttattcccgtataaaagaagtttacaacccatagggcagtcatccttcacgctacttggctggttcagactctcgtccattgaccaatattcctcactgctgcctcccgtaggagtttggaccgtgtctcagttccaatgtgggggaccttcctctcagaacccctatccatcgaaggcttggtgggccgttaccccgccaacaacctaatggaacgcatccccatcgatgaccgaaattctttaatagttccaccatgcggaagaactatgccatcgggtattaatctttctttcgaaaggctatccccgagtcatcggcaggttggatacgtgttactcacccgtgcgccggtcgccatcaaacttagcaagctaagttcatgctgcccctcgacttgcatgtgttaagcctgtagctagcgttcatcctgagccaggatcaaactcttcat

SEQ ID NO: 3 - DNA, 1525 bp, Unknown
  Bacteroides xylanisolvens GGCC_0124 16S rRNA gene
aaggaggtgttccagccgcaccttccggtacggctaccttgttacgacttagccccaattaccagttttaccctaggacgctccttgcggttacgtacttcaggtacccccggctttcatggcttgacgggcggtgtgtacaaggcccgggaacgtattcaccgcgccatggctgatgcgcgattactagcgaatccagcttcacgaagtcgggttgcagacttcgatccgaactgagagaggtttttgggattagcatcctgtcgccaggtagctgccttctgtaccccccattgtaacacgtgtgtagccccggacgtaagggccgtgctgatttgacgtcatccccaccttcctcacatcttacgacggcagtctctctagagtcctcagcatgacctgttagtaactaaagataagggttgcgctcgttatggcacttaagccgacacctcacggcacgagctgacgacaaccatgcagcaccttcacatttgccttgcggctaacctgtttccagattattcaaatgcaatttaagcccgggtaaggttcctcgcgtatcatcgaattaaaccacatgttcctccgcttgtgcgggcccccgtcaattcctttgagtttcaccgttgccggcgtactccccaggtggaatacttaatgctttcgcttggccgcttactgtatatcgcaaacagcgagtattcatcgtttactgtgtggactaccagggtatctaatcctgtttgatacccacactttcgagcatcagtgtcagttgcagtctagtgagctgccttcgcaatcggagttcttcgtgatatctaagcatttcaccgctacaccacgaattccgcccacctctactgtactcaagactgccagtttcaactgcaattttacggttgagccgcaaactttcacaactgacttaacaatccacctacgctccctttaaacccaataaatccggataacgctcggatcctccgtattaccgcggctgctggcacggagttagccgatccttattcatatggtacatacaaaattccacacgtggaatactttattcccatataaaagaagtttacaacccatagggcagtcatccttcacgctacttggctggttcagactctcgtccattgaccaatattcctcactgctgcctcccgtaggagtttggaccgtgtctcagttccaatgtgggggaccttcctctcagaacccctatccatcgtagtcttggtgggccgttaccccgccaacaaactaatggaacgcatccccatcgataaccgaaattctttaatagtaaaaccatgcggttttaatataccatcggatattaatctttctttcgaaaggctatccccgagttatcggcaggttggatacgtgttactcacccgtgcgccggtcgccatctttagtttgcaagcaaactaaaatgctgcccctcgacttgcatgtgttaagcctgtagctagcgttcatcctgagccaggatcaaactcttcat

SEQ ID NO: 4 - DNA, 947 bp, Unknown
  Barnesiella species GGCC_0306 16S rRNA gene
tgatgcgcgattactagcgatccagcttcatggagtcgagttgcagactccaatccgaactgagacaggctttcgagattcgctccacattgctgtgtggctgccctctgtacctgccattgtaacacgtgtgtcgccccggacgtaagggccgtgctgatttgacgtcatccccaccttcctcgcagcttgcgctggcagtctcgacagagtcctcagctttacctgttagtaactgtcgataggggttgcgctcgttatggcacttaagccgacacctcacggcacgagctgacgacaaccatgcagcacctcgacacagaccattgctggagacagactttcatctgctgtcctgtgtcgtttgagcccgggtaaggttcctcgcgtatcatcgaattaaaccacatgttcctccgcttgtgcgggcccccgtcaattcctttgagtttcaccgttgccggcgtactccccaggtggaatacttaacgctttcgctgtaccgcttacattgtatcgcaaacagttagtattcatcgtttactgcgtggactaccagggtatctaatcctgttcgatacccacgctttcgtgcctcagcgtcagttgtatcccagcaggctgccttcgcaatcggagttctgcgtgatatctatgcatttcaccgctacaccacgcattccgcctacctcatttacactctagaacgccagtttcaacggcagtgtccatgttgagcatggagatttcaccgctgacttggaaattccgcctacgcaccctttaaacccaataaatccggataacgcttgcatcctccgtattaccgcggctgctggcacggagttagccgatgctttttcttcggatacttgcaatacactacacgtagtgcactttactctccgataaaagaagtttacaacctgtaaggccgtcttccttca

SEQ ID NO: 5 - DNA, 1523 bp, Unknown
  Bitterella massiliensis GGCC_0305 16S rRNA gene
aaaggaggtgatccagccgcaccttccgatacggctaccttgttacgacttcaccccaatcaccagccccaccttcggcagcgtcccccttgcggttagactactgacttcgggtgttaccggctctcatggtgtgacgggcggtgtgtacaaggcccgggaacgtattcaccgcggcatgctgatccgcgattactagcaattccgacttcatgcaggcgagttgcagcctgcaatccgaactgagaccgcttttatagattcgctccacctcgcggtttcgctgctcattgttagcggccattgtagtacgtgtgtagcccaggtcataaagggcatgatgatttgacgtcatccccaccttcctccgttttgtcaacggcagtcccgttagagtgctcttgcgtagcaactaacagtaagggttgcgctcgttgcgggacttaacccaacatctcacgacacgagctgacgacaaccatgcaccacctgtcttaccgtccccgaagggaactcccaatctcttgggatagcgatagatgtcaagacctggtaaggtttttcgcgttgcttcgaattaaaccacatactccactgcttgtgcgggcccccgtcaattcctttgagtttcaaccttgcggtcgtactccccaggtggattacttattgtgttaactccggcacggagggggttatccccccacacctagtaatcatcgtttacggcatggactaccagggtatctaatcctgtttgctccccatgctttcgagcctcagcgtcagtaaaagcccagcaggccgccttcgccactggtgttcctcccgatatctacgcattccaccgctacaccgggaattccgcctgcctctacttcactcaagaaccacagttttgcgcgcagttcaggggttgagcccctgcatttcacacgcaacttatgatcccgcctacactccctttacacccagtaattccggacaacgctcgccatctacgtattaccgcggctgctggcacgtagttagccatggcttcctcctgtggtaccgtcattatcgtcccacaagacagaggtttacaatccgaaaaccttcttccctcacgcggcgtcgctgcatcagggttccccccattgtgcaatattccccactgctgcctcccgtaggagtctgggccgtgtctcagtcccaatgtggccgttcaacctctcagtccggctactgatcgtcgccttggtgggcctttaccccgccaactagctaatcagacgcgagcccatctcagagcggatcgctcctttcttgatcaggggatgtcccccaaccaccgtatgcggtattagcagtcgtttccaactgttgtcccccactccgaggcaggttgctcacgtgttactcacccgtccgccactaacaacctctctcttcctagcaagctattctaaaagaggtgtccgttcgacttgcatgtgttaggcgcgccgccagcgttcgtcctgagccaggatcaaactctttat

SEQ ID NO: 6 - DNA, 1330 bp, Unknown
  Clostridium butyricum GGCC_0151 16S rRNA gene
ctgattcgcgattactagcaactccagcttcatgtaggcgagtttcagcctacaatccgaactgagatcggttttatagttttgctcactctcgcgaggttgcatctcattgtaccgaccattgtagcacgtgtgtagccctagacataaggggcatgatgatttgacgtcatccccaccttcctcccggttaacccgggcagtctcgctagagtgctcaactaaatggtagcaactaacaataagggttgcgctcgttgcgggacttaacccaacatctcacgacacgagctgacgacaaccatgcaccacctgtcttcctgccaccgaagtggcttcctccattacagagtaattcaggagatgtcaagtctaggtaaggttcttcgcgttgcttcgaattaaaccacatgctccgctgcttgtgcgggcccccgtcaattcctttgagttttaatcttgcgaccgtactccccaggcggaatacttaatgcgttagcggcggcacagaggtcatgacaacccctacacctagtattcatcgtttacggcgtggactaccagggtatctaatcctgtttgctccccacgctttcgagcctcagtgtcagttacagtccagaaaggcgccttcgccactggtattcttcctaatctctacgcatttcaccgctacactaggaattctcctttcctctcctgcactctagatatccagtttggaatgcagcacccaggttaagcccgggtatttcacatcccacttaaatatccacctacgctccctttacgcccagtaaatccggacaacgcttgccacctacgtattaccgcggctgctggcacgtagttagccgtggcttcctccttaggtaccgtcattatcgtccctaaagacagagctttacaatccgaagaccgtcatcactcacgcggcgttgctgcatcagggtttcccccattgtgcaatattccccactgctgcctcccgtaggagtctgggccgtgtctcagtcccaatgtggccgatcaccctctcaggtcggctacgcatcgtcgccttggtgagccgttacctcaccaactagctaatgcgacgcgggtccatctcatagcggattactcctttaattgctgtaccatgcggtactacaatcttatgcggtattaatcttcctttcgaaaggctattcccctctatgaggcaggttacccacgtgttactcacccgtccgccgctaatccactcccgaaggagcttcatcgctcgacttgcatgtgttaagcacgccgccagcgttcgtcctgagccaggatcaaactctcaata

SEQ ID NO: 7 - DNA, 1532 bp, Unknown
  Clostridium scindens GGCC_0168 16S rRNA gene
aaaggaggtgatccagccgcaccttccgatacggctaccttgttacgacttcaccccagttatcggtcccaccttcgacggctccctcccttacgggttgggtcaccggcttcgggcgttactgactcccatggtgtgacgggcggtgtgtacaagacccgggaacgtattcaccgcgacattctgattcgcgattactagcgattccagcttcatgtagtcgagttgcagactacaatccgaactgagacgttatttttgggatttgcccaccctcgcgggttcgcctccctttgtttacgccattgtagcacgtgtgtagccctggtcataaggggcatgatgatttgacgtcatccccaccttcctccaggttctccctggcagtctctccagagtgcccatccataatgctggctactgaagataggggttgcgctcgttgcgggacttaacccaacatctcacgacacgagctgacgacaaccatgcaccacctgtcaccgatgttccgaagaaagagcgcgttacgcgctttggcatcgggatgtcaagatcaggtaaggttcttcgcgttgcttcgaattaaaccacatgctccaccgcttgtgcgggtccccgtcaattcctttgagtttcattcttgcgaacgtactccccaggtggactacttattgcgtttgctgcggcaccgaatggccttgccacccgacacctagtagtcatcgtttacggcgtggactaccagggtatctaatcctgtttgctccccacgctttcgagcctcaacgtcagtcatcgtccagcaggccgccttcgccactggtgttcctcctaatatctacgcatttcaccgctacactaggaattccgcctgcctctccgacactccagccacgcagttccaaatgcagtcccggggttgagccccgggctttcacatctggcttgcatcgccgtctacgctccctttacacccagtaaatccggataacgcttgccccctacgtattaccgcggctgctggcacgtagttagccggggcttcttagtcaggtaccgtcatcttcttccctgctgatagaagtttacataccgaaatacttcatccttcacgcggcgtcgctgcatcagggtttcccccattgtgcaatattccccactgctgcctcccgtaggagtctgggccgtgtctcagtcccaatgtggccggtcaccctctcaggtcggctactgatcgtcggcttggtgggccgttaccccgccaactacctaatcagacgcgggcccatcttgcaccgccggggctttggccgccgcgccatgcggcgcttcggtcttatgcggtattagcagccatttctggctgttatcccccagtgcaaggcaggttgcccacgcgttactcacccgtccgccactcagtcgcaaggctcctcgttccgaagaagtcggggccaggcgcttcgttcgacttgcatgtgttaggcacgccgccagcgttcatcctgagccaggatcaaactctcgtt

SEQ ID NO: 8 - DNA, 1527 bp, Unknown
  Clostridium symbiosum GGCC_0272 16S rRNA gene
aaaggaggtgatccagccgcaccttccgatacggctaccttgttacgacttcaccccagttatcggtcccgccttcggcagctccctccttgcggttgggtcactgacttcgggcgttactgactcccatggtgtgacgggcggtgtgtacaagacccgggaacgtattcaccgcgacattctgattcgcgattactagcgattccagcttcgtgcaggcgagttgcagcctgcagtccgaactgagacgttatttttgagatttgctccacctcgcggtcttgcttctctttgtttacgccattgtagcacgtgtgtagcccagatcataaggggcatgatgatttgacgtcatccccaccttcctccaggttatccctggcagtctcccaagagttcccggccgaaccgctggctacttagaataagggttgcgctcgttgcgggacttaacccaacatctcacgacacgagctgacgacaaccatgcaccacctgtcttctccgccccgaagggaaggggacgttactcccccgtcggatcgatgtcaagacctggtaaggttcttcgcgttgcttcgaattaaaccacatgctccaccgcttgtgcgggtccccgtcaattcctttgagtttcattcttgcgaacgtactccccaggtggaatacttactgcgtttgcgacggcaccgaagagctttgctccccaacacctagtattcatcgtttacggcgtggactaccagggtatctaatcctgtttgctccccacgctttcgagcctcaacgtcagttatcgtccagtaagtcgccttcgccactggtgttcctcctaatatctacgcatttcaccgctacactaggaattccacttacctctccgacactccagttaaacagtttccaaagcagtcccgcagttgagccgcgggctttcacttcagacttgctttaccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcttcactcacgcggcgtcgctgcatcaggctttcgcccattgtgcaatattccccactgctgcctcccgtaggagtttgggccgtgtctcagtcccaatgtggccggtcaccctctcaggtcggctactgatcgtcgccttggtaagccgttaccttaccaactagctaatcagacgcgggtccatcttgtaccaccggagtttttcacactgtatcatgcgatactgtgcgcttatgcggtattagcagtcatttctaactgttgtcccccagtacaaggcaggttacccacgcgttactcacccgtccgccactcagtcaattcaacttccatccgaaaacttccgttaaatcgcttcgttcgacttgcatgtgttaggcacgccgccagcgttcatcctgagccaggatcaaactctcat

SEQ ID NO: 9 - DNA, 1522 bp, Unknown
  Eubacterium limosum GGCC_0197 16S rRNA gene
aaaggaggtgatccagccgcaccttccgatacggctaccttgttacgacttcaccccaattactagccccaccttcgactgctgagtccttgcggttctctcacaggcttcgggtgttgccaactctcgtggtgtgacgggcggtgtgtacaagacccgggaacgcattcaccgcggcattctgatccgcgattactagcaactccaacttcatgcaggcgagttgcagcctgcaatccgaactgggatctgttttaagggatttgcttcacctcgcggcttcgcggccctctgttcagaccattgtagcacgtgtgtagcccaggtcataaggggcatgatgatttgacgtcatccccaccttcctccgtattgtctacggcagtccctctagagtgcccaactgaatgctggcaactaaaggcaggggttgcgctcgttgcgggacttaacccaacatctcacgacacgagctgacgacaaccatgcaccacctgtctctctgttcccgaaggaaacttcctatctctaggctcgtcagaggatgtcaagacctggtaaggttcttcgcgttgcttcgaattaaaccacatgctccgctgcttgtgcgggtccccgtcaattcctttgagtttcaaccttgcggtcgtactccccaggcggaatgcttattgtgttaactgcggcactgagtttccccaacacctagcattcatcgtttacggcgtggactaccagggtatctaatcctgttcgctccccacgctttcgcacctcagcgtcagtatttgtccagcaagccgccttcgccactggtgttcctcctaatatctacgcatttcaccgctacactaggaattccacttgcctctccaatactcaagtctaccagtttccaatgcacttcaccggttgagccggtacctttcacatcagacttaatagaccgcctacgcgccctttacgcccagtcattccggacaacgcttgtcccctacgtattaccgcggctgctggcacgtagttagccgggacttcctcattgggtaccgtcatgttttcttccccaataacagagctttacgatccgaaaaccttcttcactcacgcggtattgctgcgtcagggttgcccccattgcgcaatattccccactgctgcctcccgtaggagtctggaccgtgtctcagttccagtgtgaccgttcgccctctcagaccggttacccatcgtcgccttggtgggctgttatctcaccaactagctaatgggacgcgggtccatcctatggcaccggagttttcatgatcttgccatgcgacaaaaccataatataaggctttactcccagtttcccgaggctattcctttccatagggcaggttacccacgcgttactcacccgttcgccactttccagttctaatttcacccgaaggatcattcaaaaccttctcgttcgacttgcatgtgttaagcataccgccagcgttcgtcctgagccaggatcaaactctcaata

SEQ ID NO: 10 - DNA, 951 bp, Unknown
  Extibacter species GGCC_0201 16S rRNA gene
ctgattcgcgattactagcgattccagcttcatgtagtcgggttgcagactacaatccgaactgggacgttatttttgggattcgcttgccctcgcggggtcgcctccctctgtttacgccattgtagcacgtgtgtagccctgatcataaggggcatgatgatttgacgtcatccccaccttcctccaggttctccctggcagtctctctagagtgcccagcctcacctgctggctactaaagataggggttgcgctcgttgcgggacttaacccaacatctcacgacacgagctgacgacaaccatgcaccacctgtctcccctgtcccgtaggaaaggacacgttactgtccggtcagggggatgtcaagaccaggtaaggttcttcgcgttgcttcgaattaaaccacatgctccaccgcttgtgcgggtccccgtcaattcctttgagtttcattcttgcgaacgtactccccaggtggattacttaatgcgttggctgcggcaccgggaagctttgcttcccgacacctagtaatcatcgtttacggcgtggactaccagggtatctaatcctgtttgctccccacgctttcgagcctcaacgtcagtcatcgtccagcaagccgccttcgccactggtgttcctcctaatatctacgcatttcaccgctacactaggaattccacttgcctctccgacactctagctccacagtttccaaagcagtcccggggttgagccccgggctttcactccagacttgcagagccgtctacgctccctttacacccagtaaatccggataacgcttgccccctacgtattaccgcggctgctggcacgtagttagccggggcttcttagtcaggtaccgtcatcttcttccctgctgatagaagtttacataccgaaatacttcatccttcacgcggcgtcgctgcatca

SEQ ID NO: 11 - DNA, 1530 bp, Unknown
  Intestinimonas butyriciproducens GGCC_0179 16S rRNA gene
aaaggaggtgatccagccgcaccttccgatacggctaccttgttacgacttcaccccaattatcgaacccaccttcggccgcgcccccattgctgttaggctacggacttcgggtgttcccgactctcatggtgtgacgggcggtgtgtacaaggcccgggaacgtattcaccgcggcatgctgatccgcgattactagcgattccgacttcatacaggcgagtttcagcctgcaatccgaactgggacggcttttagggatttgctccacctcgcggctttgcttccctctgttgaccgccattgtagtacgtgtgtggcccaggacataaggggcatgatgatttgacgtcgtccccaccttcctccgttttgtcaacggcagtctcgctagagtgctcttgcgtagcaactaacaataagggttgcgctcgttgcgggacttaacccaacatctcacgacacgagctgacgacaaccatgcaccacctgtctctactttccccgaagggcaccttatgcatctctgcttcgttagtaggatgtcaagtcctggtaaggttcttcgcgttgcttcgaattaaaccacatactccaccgcttgtgcgggcccccgtcaattcctttgagtttcaaccttgcgatcgtactccccaggtgggatacttattgtgttaactgcggcacggagggggtcagaccccccacacctagtatccatcgtttacggcgtggactaccagggtatctaatcctgtttgctccccacgctttcgcgcctcagcgtcagttaatgtccagcaggccgccttcgccactggtgttcctccctatatctacgcatttcaccgctacacagggaattccgcctgcctctccatcactcaagaagtacagtttcaaatgcaggctatgggttgagcccatagttttcacatctgacttgcactcccgcctacacgccctttacacccagtaaatccggataacgcttgccacctacgtattaccgcggctgctggcacgtagttagccgtggcttattcgtcaggtaccgtcacttgcttcgtccctgacaaaagaagtttacaacccgagggccttcttccttcacgcggcgttgctgggtcaggctttcgcccattgcccaatattccccactgctgcctcccgtaggagtctgggccgtgtctcagtcccaatgtggccggccaacctctcagtccggctactgatcgtcgccttggtgagccattacctcaccaactagctaatcagacgcgagtccatctcagagcgataaatctttggtatccagaaccatgcgatccagatacatcatgcggtattagcagccgtttccggctgttattccccactccaaggcaggttactcacgcgttactcacccgtccgccactaagcattccctttcgttgtccgaaaactccgtcaggggtgctccgttcgacttgcatgtgttaagcacgccgccagcgttcatcctgagccaggatcaaactctcaata

(19) United States
(12) Patent Application Publication
(10) Pub. No.: US 2023/0381247 A1
(43) Pub. Date: Nov. 30, 2023

(54) MICROBIAL-BASED COMPOSITIONS FOR SYSTEMIC INFLAMMATION CONTROL

(71) Applicant: Gusto Global, LLC, Charlotte, NC (US)

(72) Inventors: Daniel van der Lelie, Chapel Hill, NC (US); Safiyh Taghavi, Chapel Hill, NC (US)

(21) Appl. No.: 18/032,745

(22) PCT Filed: Oct. 14, 2021
(86) PCT No.: PCT/US2021/054944

§ 371 Date: Apr. 19, 2023

Related U.S. Application Data
(60) Provisional application No. 63/094,655, filed on Oct. 21, 2020.

(51) Int. Cl. A61K 35/742; C12N 1/20; A61K 45/06; A61P 29/00

(52) U.S. Cl. CPC A61K 35/742; C12N 1/20; A61K 45/06; A61P 29/00; C12R 2001/145

(57) ABSTRACT

Compositions containing live cultures of 11 separately isolated bacterial strains (the bacterial consortium containing all 11 strains designated GUT-108) are provided for treating conditions of systemic inflammation including, to name a few, Inflammatory Bowel Diseases, type-2 diabetes, metabolic syndrome and obesity, and aging-related conditions of chronic inflammation. In a mouse model of chronic immune-mediated colitis, therapeutic application of GUT-108 reversed the established inflammation in the cecum. In addition, the treatment with GUT-108 unexpectedly resulted in restoration of a healthy gut microbiome in the mice containing an abundance of beneficial Clostridium strains not part of GUT-108, and a decrease in abundance of the applied pathogenic Enterobacteriaceae bacteria from 5% to below 0.5%. An eight-fold decrease in the levels of pathogenic Clostridium perfringens was also observed after treatment with GUT-108, indicating the efficacy of GUT-108 in treating inflammation caused by pathogenic species of the Enterobacteriaceae or pathogenic species of Clostridium.

### CROSS-REFERENCE TO RELATED APPLICATIONS

This application claims the benefit of priority of U.S. provisional patent application no. 63/094,655, filed on Oct. 21, 2020, the disclosure of which is incorporated herein by this reference in its entirety.

### TECHNICAL FIELD

The presently disclosed subject matter relates to microbial-based compositions and methods for overcoming conditions whose pathogenesis is characterized by dysbiosis-mediated chronic intestinal inflammation and increased mucosal permeability.

### BACKGROUND

Conditions of chronic inflammation of the gastrointestinal tract have been linked to a range of conditions, such as Inflammatory Bowel Diseases (IBD) like ulcerative colitis and Crohn's disease, Pouchitis, Irritable Bowel Syndrome (IBS), graft versus host disease, Parkinson's disease, ALS, multiple sclerosis, systemic lupus erythematosus, Ankylosing Spondylitis, asthma, food allergies, fatty liver, Primary Sclerosing Cholangitis as a comorbidity from IBD, hepatic encephalopathy, type-2 diabetes, metabolic syndrome and obesity, Plaque Psoriasis, Psoriatic Arthritis, and aging related conditions of chronic inflammation, each of which have been shown to have an underlying microbially-mediated inflammatory component.

Chronic intestinal inflammation can be induced by multiple exogenous and endogenous signals and mediated by multiple immune and nonimmune cells. As reviewed by De Souza and Flocchi (2016) and Sartor and Wu (2017), the epithelial translocation of exogenous substances, including dietary antigens, pathogenic microorganisms, xenobiotics including antibiotics, or a combination of them, can trigger an initial response mediated primarily by immune cells that initiates mucosal inflammation of the gastrointestinal tract. This primary inflammatory response induces a variety of processes, which trigger a secondary inflammatory response, eventually resulting in a self-sustaining cycle of chronic inflammation of the gastrointestinal tract with increased mucosal permeability, tissue damage, cell death and leaky gut symptoms, as well as dysbiosis of the gut microbiome. The dysbiotic gut microbiome creates an ideal environment for the establishment of (opportunistic) pathogens, which in turn will also contribute to the severity of the inflammation and its clinical symptoms.

An example of such condition is IBD, which encompasses two main clinical disorders: Crohn's disease and Ulcerative Colitis. Drug treatments for IBD primarily focus on inflammation control through anti-inflammatory and immunosuppressive therapies. Examples of some of the most successful biological drugs for treatment of IBD include corticosteroids for early stages of disease onset, and a range of drugs for treating advanced stages of the disease such as REMICADE (Infliximab; manufactured and sold by Johnson & Johnson subsidiary Janssen Biotech), HUMIRA (Adalimumab; manufactured and sold by AbbVie), Stelara (ustekinumab; manufactured and sold by Johnson & Johnson subsidiary Janssen Biotech), and ENTYVIO (vedolizumab; developed by Millennium Pharmaceuticals, Inc. and sold by Takeda), which target the body's immune response through key pathways by neutralizing a single cytokine or molecule and induce immunosuppression to control the inflammatory process. However, these and other immune modulating drugs only work on select patient cohorts and have multiple side effects, including the increased risk for serious and potentially life-threatening infections and neoplastic safety concerns. In addition, none of these drugs addresses the upstream conditions that contribute to the chronic inflammation, including increased mucosal permeability, the leaky gut and the dysbiotic pro-inflammatory gut microbiome.

To treat IBD associated infections and septic complications such as abscesses, antibiotics, particularly ciprofloxacin and metronidazole, are commonly prescribed; however, treatment with antibiotics is often unsuccessful (Gevers et al, 2017; Sartor and Wu, 2017). Furthermore, the application of broad-spectrum antibiotics can be detrimental to a healthy gut microbiome by non-selectively eliminating bacterial populations, including protective species that prevent overgrowth of opportunistic pathogens such as Clostridium difficile. As such, broad spectrum antibiotics may further contribute to the dysbiotic status of the gut microbiome in IBD patients, with additional negative consequences on the regulation of the innate immune response and the conditions of chronic inflammation.

As an alternative to drugs, microbiome inspired therapeutics are being developed for the treatment of IBD and other conditions linked to chronic inflammation of the gastrointestinal tract. The focus of this work has been on the use of strains belonging to the Clostridium clusters IV and XIVa. Strains of Clostridium cluster IV and XIVa were found to be successful in decreasing inflammation and necrosis in rodent IBD models (Sokol et al, 2008; Eeckhaut et al, 2009). Van Immerseel et al (2010) suggested the use of Clostridial cluster IV and XIVa strains as preventive and therapeutic probiotics for IBD. Using germ-free (GF) mice inoculated with healthy human fecal material pretreated with chloroform to enrich for spore-forming bacteria, Honda et al developed a 17-strain consortium for IBD treatment (see, e.g., PCT application no. WO2011151941A1—Composition having activity of inducing proliferation or accumulation of regulatory T cells; Furusawa et al, 2013; Honda et al, 2015). This consortium of bacterial strains comprised of Clostridium cluster IV, XIVa and XVIII strains, induced the recruitment/accumulation of regulatory T cells (Treg cells) in the colon. These Treg cells suppressed proliferation of effector T cells, which in turn lowered the inflammation response by the immune system in the gut (Atarashi et al, 2013). However, these approaches have several disadvantages. First, with enrichment-based approaches, the outcome is defined by the stool sample used in the enrichment, with different samples resulting in different consortia. In addition, undesirable strains or strains harboring undesirable functions associated with safety risks including virulence factors and transferable antibiotic resistance functions can be present. Finally, other bacterial species besides spore-forming Clostridium bacteria are required for optimal engraftment and therapeutic performance in the dysbiotic gut environment of patients with intestinal inflammation.

Another commonly used approach for discovery of microbiome-based therapeutics has been to compare the microbiomes of healthy subjects and patients suffering from a specific condition to identify microorganisms lacking or under-represented in the patients. This information is then used to propose a therapeutic formulation to replenish the microorganisms that are lacking or under-represented. This approach has been used for conditions where a role of the microbiome has been implied, including immune dysregulation such as IBD (Atarashi et al, 2013; Nrushima et al, 2014; Halfvarson et al, 2017), food allergies (Canani et al, 2016) and asthma (Canani et al, 2017), neurological disorders such as Alzheimer's Disease (Bhattacharjee and Lukiw, 2013), Autism Spectrum Disorder (Li and Zhou, 2016; Vuong and Hsiao, 2017), dementia (Moos et al, 2016), peri-natal/post-partum depression (Rogers et al, 2016), oncology related conditions such as colon cancer (and other GI cancers) (Schwabe and Jobin, 2013; Sun and Kato, 2016), metabolic disorders (Boulange et al, 2016) including Type-1 and Type-2 diabetes (Paun and Danska, 2016), and recurrent infections with Clostridium difficile (Seekatz, 2016). However, there is currently no FDA approved microbiome-based therapeutic available for the treatment of any of these conditions, and the role of the compositional make-up of the gut microbiome and the role of individual strains within a particular species of gut bacteria in the positive outcome of inflammatory disorders remains unclear.

There is a lack of approved microbiome-based therapeutics for conditions involving chronic inflammation and a lack of clarity around how to design or obtain effective microbiome-based therapeutics. In addition, common therapeutics for conditions involving chronic inflammation (e.g., corticosteroids, antibiotics, immunomodulators, REMICADE, HUMIRA, ENTYVIO, STELARA, TALTZ, XELJANZ, and biosimilars such as RENFLEXIS (Infliximab-abda; Samsung and marketing partner Merck) suffer from serious side effects including dysbiosis of the gut microbiome, risk of serious and potentially life-threatening infection, and increased risks of malignancies. Furthermore, interference with the immune system in subjects having an inflammatory disorder with an underlying microbiome component will not result in sustainable remission unless gut microbiome dysbiosis is addressed.

Thus, there remains an unmet need for improved compositions and methods to treat conditions involving chronic inflammation. The presently disclosed subject matter provides such improved compositions and methods that do not suffer from these negative side effects.

### SUMMARY

In one embodiment of the present disclosure, a composition is provided for use in a method of benefiting the health of an animal or a human including a biologically pure culture of each of: Akkermansia species—like strain GGCC_0220, Bacteroides uniformis—like strain GGCC_0301, Bacteroides xylanisolvens—like strain GGCC_0124, Barnesiella species—like strain GGCC_0306, Bitterella massiliensis—like strain GGCC_0305, Clostridium butyricum—like strain GGCC_0151, Clostridium scindens—like strain GGCC_0168, Clostridium symbiosum—like strain GGCC_0272, Eubacterium callanderi—like strain GGCC_0197, Extibacter species—like strain GGCC_0201 or Intestinimonas butyriciproducens—like strain GGCC_0179.

The bacterial consortium comprising all 11 of the above bacterial strains is designated “GUT-108”.

In one embodiment of the present disclosure, a composition for use in a method of benefiting the health of an animal or a human is provided including a biologically pure culture of two or more of: Akkermansia species—like strain GGCC_0220, Bacteroides uniformis—like strain GGCC_0301, Bacteroides xylanisolvens—like strain GGCC_0124, Barnesiella species—like strain GGCC_0306, Bitterella massiliensis—like strain GGCC_0305, Clostridium butyricum—like strain GGCC_0151, Clostridium scindens—like strain GGCC_0168, Clostridium symbiosum—like strain GGCC_0272, Eubacterium callanderi—like strain GGCC_0197, Extibacter species—like strain GGCC_0201 or Intestinimonas butyriciproducens—like strain GGCC_0179.

In one embodiment of the present disclosure, a composition for use in a method of benefiting the health of an animal or a human is provided comprising a biologically pure culture of two or more of a bacterium having 97% sequence identity to the 16S rRNA gene sequence of: Akkermansia species—like strain GGCC_0220 (SEQ ID NO: 1), Bacteroides uniformis—like strain GGCC_0301 (SEQ ID NO: 2), Bacteroides xylanisolvens—like strain GGCC_0124 (SEQ ID NO: 3), Barnesiella species—like strain GGCC_0306 (SEQ ID NO: 4), Bitterella massiliensis—like strain GGCC_0305 (SEQ ID NO: 5), Clostridium butyricum—like strain GGCC_0151 (SEQ ID NO: 6), Clostridium scindens— like strain GGCC_0168 (SEQ ID NO: 7), Clostridium symbiosum—like strain GGCC_0272 (SEQ ID NO: 8), Eubacterium callanderi—like strain GGCC_0197 (SEQ ID NO: 9), Extibacter species—like strain GGCC_0201 (SEQ ID NO: 10) or Intestinimonas butyriciproducens—like strain GGCC_0179 (SEQ ID NO: 11).

In one embodiment of the present disclosure, a composition for use in a method of benefiting the health of an animal or a human is provided comprising two or more of:
a) a biologically pure culture of a bacterium having 97% sequence identity to the 16S rRNA gene sequence of Akkermansia species—like strain GGCC_0220 (SEQ ID NO: 1), said bacterium comprising genetic material encoding for synthesis of propionate, synthesis of 4-amino-butyrate (gamma-aminobutyric acid; GABA), and synthesis of indole or indole-containing secondary metabolites;
b) a biologically pure culture of a bacterium having 97% sequence identity to the 16S rRNA gene sequence of Bacteroides uniformis—like strain GGCC_0301 (SEQ ID NO: 2), said bacterium comprising genetic material encoding for synthesis of propionate, synthesis of GABA, and synthesis of indole or indole-containing secondary metabolites;
c) a biologically pure culture of a bacterium having 97% sequence identity to the 16S rRNA gene sequence of Bacteroides xylanisolvens—like strain GGCC_0124 (SEQ ID NO: 3), said bacterium comprising genetic material encoding for synthesis of propionate, synthesis of GABA, and synthesis of indole or indole-containing secondary metabolites;
d) a biologically pure culture of a bacterium having 99% sequence identity to the 16S rRNA gene sequence of Barnesiella species—like strain GGCC_0306 (SEQ ID NO: 4), said bacterium comprising genetic material encoding for synthesis of propionate, and synthesis of GABA;
e) a biologically pure culture of a bacterium having 97% sequence identity to the 16S rRNA gene sequence of Bitterella massiliensis— like strain GGCC_0305 (SEQ ID NO: 5), said bacterium comprising genetic material encoding for 3α-hydroxy steroid dehydrogenase and 3β-hydroxy steroid dehydrogenase;
f) a biologically pure culture of a bacterium 97% sequence identity to the 16S rRNA gene sequence of Clostridium butyricum— like strain GGCC_0151 (SEQ ID NO: 6), said bacterium comprising genetic material encoding for synthesis of butyrate;
g) a biologically pure culture of a bacterium having 97% sequence identity to the 16S rRNA gene sequence of Clostridium scindens—like strain GGCC_0168 (SEQ ID NO: 7), said bacterium comprising genetic material encoding for bile acid 7-alpha-dehydratase activity;
h) a biologically pure culture of a bacterium having 97% sequence identity to the 16S rRNA gene sequence of Clostridium symbiosum—like strain GGCC_0272 (SEQ ID NO: 8), said bacterium comprising genetic material encoding for synthesis of butyrate
i) a biologically pure culture of a bacterium having 97% sequence identity to the 16S rRNA gene sequence of Eubacterium callanderi—like strain GGCC_0197 (SEQ ID NO: 9), said bacterium comprising genetic material encoding for synthesis of butyrate, synthesis of GABA, and bile acid 7-alpha-dehydratase activity;
j) a biologically pure culture of a bacterium having 97% sequence identity to the 16S rRNA gene sequence of Extibacter species—like strain GGCC_0201 (SEQ ID NO: 10), said bacterium comprising genetic material encoding for bile acid 7-alpha-dehydratase activity; and
k) a biologically pure culture of a bacterium having 97% sequence identity to the 16S rRNA gene sequence of Intestinimonas butyriciproducens—like strain GGCC_0179 (SEQ ID NO: 11) said bacterium comprising genetic material encoding for synthesis of butyrate.

The compositions of the present disclosure can include three, four, five, six, seven, eight, nine, ten, or eleven of the biologically pure cultures of the strains of GUT-108.

The compositions of the present disclosure can include one or more strains having 95%, 96%, 97%, 98%, or 99% sequence identity to the 16S rRNA gene sequence of any one of the strains of GUT-108.

The compositions of the present disclosure can include one or more strains having 95%, 96%, 97%, 98%, or 99% sequence identity to the 16S rRNA gene sequence of any one of the strains of GUT-108, wherein each of the strains also encodes for the functionalities designated in TABLE 1.

In one embodiment of the present disclosure, a composition for use in a method of benefiting the health of an animal or a human is provided comprising a biologically pure culture of each of: Akkermansia species—like strain GGCC_0220, Bacteroides uniformis—like strain GGCC_0301, Bacteroides xylanisolvens—like strain GGCC_0124, Clostridium butyricum—like strain GGCC_0151, Clostridium scindens—like strain GGCC_0168, Clostridium symbiosum—like strain GGCC_0272, Eubacterium callanderi—like strain GGCC_0197, Extibacter species—like strain GGCC_0201 or Intestinimonas butyriciproducens—like strain GGCC_0179.

In one embodiment of the present disclosure, a composition for use in a method of benefiting the health of an animal or a human is provided comprising a biologically pure culture of each of a bacterium having 97% sequence identity to the 16S rRNA gene sequence of: Akkermansia species—like strain GGCC_0220 (SEQ ID NO: 1), Bacteroides uniformis—like strain GGCC_0301 (SEQ ID NO: 2), Bacteroides xylanisolvens—like strain GGCC_0124 (SEQ ID NO: 3), Clostridium butyricum—like strain GGCC_0151 (SEQ ID NO: 6), Clostridium scindens— like strain GGCC_0168 (SEQ ID NO: 7), Clostridium symbiosum—like strain GGCC_0272 (SEQ ID NO: 8), Eubacterium callanderi—like strain GGCC_0197 (SEQ ID NO: 9), Extibacter species—like strain GGCC_0201 (SEQ ID NO: 10) or Intestinimonas butyriciproducens—like strain GGCC_0179 (SEQ ID NO: 11).

In one embodiment of the present disclosure, a composition for use in a method of benefiting the health of an animal or a human is provided comprising each of:
a) a biologically pure culture of a bacterium having 97% sequence identity to the 16S rRNA gene sequence of Akkermansia species—like strain GGCC_0220 (SEQ ID NO: 1), said bacterium comprising genetic material encoding for synthesis of propionate, synthesis of 4-amino-butyrate (gamma-aminobutyric acid; GABA), and synthesis of indole or indole-containing secondary metabolites;
b) a biologically pure culture of a bacterium having 97% sequence identity to the 16S rRNA gene sequence of Bacteroides uniformis—like strain GGCC_0301 (SEQ ID NO: 2), said bacterium comprising genetic material encoding for synthesis of propionate, synthesis of GABA, and synthesis of indole or indole-containing secondary metabolites;
c) a biologically pure culture of a bacterium having 97% sequence identity to the 16S rRNA gene sequence of Bacteroides xylanisolvens—like strain GGCC_0124 (SEQ ID NO: 3), said bacterium comprising genetic material encoding for synthesis of propionate, synthesis of GABA, and synthesis of indole or indole-containing secondary metabolites;
d) a biologically pure culture of a bacterium 97% sequence identity to the 16S rRNA gene sequence of Clostridium butyricum—like strain GGCC_0151 (SEQ ID NO: 6), said bacterium comprising genetic material encoding for synthesis of butyrate;
e) a biologically pure culture of a bacterium having 97% sequence identity to the 16S rRNA gene sequence of Clostridium scindens—like strain GGCC_0168 (SEQ ID NO: 7), said bacterium comprising genetic material encoding for bile acid 7-alpha-dehydratase activity;
f) a biologically pure culture of a bacterium having 97% sequence identity to the 16S rRNA gene sequence of Clostridium symbiosum—like strain GGCC_0272 (SEQ ID NO: 8), said bacterium comprising genetic material encoding for synthesis of butyrate;
g) a biologically pure culture of a bacterium having 97% sequence identity to the 16S rRNA gene sequence of Eubacterium callanderi—like strain GGCC_0197 (SEQ ID NO: 9), said bacterium comprising genetic material encoding for synthesis of butyrate, synthesis of GABA, and bile acid 7-alpha-dehydratase activity;
h) a biologically pure culture of a bacterium having 97% sequence identity to the 16S rRNA gene sequence of Extibacter species—like strain GGCC_0201 (SEQ ID NO: 10), said bacterium comprising genetic material encoding for bile acid 7-alpha-dehydratase activity; and
i) a biologically pure culture of a bacterium having 97% sequence identity to the 16S rRNA gene sequence of Intestinimonas butyriciproducens—like strain GGCC_0179 (SEQ ID NO: 11) said bacterium comprising genetic material encoding for synthesis of butyrate.

The compositions of the present disclosure may be formulated as pharmaceutical compositions. The compositions of the present disclosure may include one or more pharmaceutically acceptable carriers. The compositions of the present disclosure may be formulated for delivery to the intestine. In the compositions of the present disclosure, the bacteria may be in the form of spores or vegetative cells or a combination thereof. In the compositions of the present disclosure, the bacteria may be in the form of freeze-dried spores or vegetative cells or a combination thereof.

In one embodiment, a method is provided for treating or preventing an inflammatory disorder in a subject or for improving the microbiome of a subject, the method including administering to the subject a composition of the present disclosure, wherein the administering results in one or both of a decrease in inflammation or an improvement in the health of the subject's microbiome.

In one embodiment, a method is provided for treating or preventing an inflammatory disorder in a subject or for improving the microbiome of a subject, the method including administering to the subject a composition comprising a biologically pure culture of two or more of the strains of the GUT-108 consortium.

In the methods of the present disclosure for treating or preventing an inflammatory disorder in a subject or for improving the microbiome of a subject, the compositions can include one or more strains having 95%, 96%, 97%, 98%, or 99% sequence identity to the 16S rRNA gene sequence of any one of the strains of GUT-108.

In the methods of the present disclosure for treating or preventing an inflammatory disorder in a subject or for improving the microbiome of a subject, the compositions can include one or more strains having 95%, 96%, 97%, 98%, or 99% sequence identity to the 16S rRNA gene sequence of any one of the strains of GUT-108, wherein each of the strains also encodes for the functionalities designated in TABLE 1.

In the methods of the present disclosure, the inflammatory disorder can include ulcerative colitis, Crohn's disease, Ankylosing Spondylitis, Plaque Psoriasis, Psoriatic Arthritis or Pouchitis.

In the methods of the present disclosure, the inflammatory disorder can include Alzheimer's and Non-Alzheimer's disease related dementia, Parkinson's disease, multiple sclerosis, Type-2 diabetes, obesity, hepatic steatosis, or aging related cardiovascular or neurologic conditions.

In the methods of the present disclosure, the inflammatory disorder can include infection or overpopulation with one or a combination of pathogenic members of the Enterobacteriaceae or pathogenic species of Clostridium.

In the methods of the present disclosure for treating inflammation, the composition can be administered in combination with one or both of a corticosteroid and one or a combination of the drugs listed in Table 3.

In the methods of the present disclosure, the composition can be administered in combination with one or both of a food supplement or a prebiotic.

In one embodiment, a method is provided for monitoring systemic inflammation in a subject, including: measuring a level of one or a combination of a secondary metabolite biomarker in a stool sample of a subject, wherein the biomarker is selected from the group consisting of propionate, indole and its derivatives indole propionate (IPA) and indole acetate (IAA), and secondary bile acids deoxycholic acid (DCA) and lithocholic acid (LCA), wherein an increased level of the one or a combination of biomarkers relative to a control is an indication of a decrease in systemic inflammation in the subject.

### BRIEF DESCRIPTION OF THE DRAWINGS

The foregoing aspects and other features of the invention are explained in the following description, taken in connection with the accompanying drawings.

FIG. 1A is a schematic showing the effect on the gut microbiome community composition, of gnotobiotic mice inoculated with GUT-108 for 2 weeks. Il10⁺/^{eGFP} VertX reporter mice were inoculated via gavage with GUT-108 (2×10⁺⁷ cfu/strain), and after 2 weeks community composition of the gut microbiome was determined on cecal contents using qPCR with species specific primers. The average community composition (circle diagrams) for five animals per treatment are presented. The lower detection limit for qPCR was >0.0001%.

FIG. 1B is a schematic showing the effect on the gut microbiome community composition, of gnotobiotic mice inoculated with GUT-108 for 2 weeks. Il10^{-/-} mice were inoculated via gavage with GUT-108 (2×10⁺⁷ cfu/strain), and after 2 weeks community composition of the gut microbiome was determined on cecal contents using qPCR with species specific primers. The average community composition (circle diagrams) for five animals per treatment are presented. The lower detection limit for qPCR was >0.0001%.

FIG. 2A is a representative histology image of cecum tissue from an ex-germ-free Il10^{-/-} mouse model of IBD treated with PBS as a control. Bar in upper right corner indicates 100 μm. The experiment was repeated independently with similar results for each animal in the PBS (N=8) group.

FIG. 2B is a representative histology image of cecum tissue from an ex-germ-free Il10^{-/-} mouse model of IBD treated with GUT-108 showing that GUT-108 did not induce colitis in the susceptible mice. Bar in upper right corner indicates 100 μm. The experiment was repeated independently with similar results for each animal in the GUT-108 (N=11) group.

FIG. 3A is a graph showing metabolite analysis of fecal material from Il10^{-/-} mice inoculated with GUT-108 to confirm the successful restoration of bacterial functions for the synthesis of acetate. Two-Way ANOVA with Sidak's multiple comparison test. Bar indicates mean+/−SE. ***P<0.001, ****P<0.0005, NS: not significant. N=8/group or N=11/group, for PBS and GUT-108 treatment, respectively.

FIG. 3B is a graph showing metabolite analysis of fecal material from Il10^{-/-} mice inoculated with GUT-108 to confirm the successful restoration of bacterial functions for the synthesis of propionate as described in FIG. 3A.

FIG. 3C is a graph showing metabolite analysis of fecal material from Il10^{-/-} mice inoculated with GUT-108 to confirm the successful restoration of bacterial functions for the synthesis of butyrate, as described in FIG. 3A.

FIG. 3D is a graph showing metabolite analysis of fecal material from Il10^{-/-} mice inoculated with GUT-108 to confirm the successful restoration of bacterial functions for the synthesis of IAA as described in FIG. 3A.

FIG. 4 is a graph showing the differences in cell counts per 10⁶ lamina propria cells of various IL10 synthesizing immune cell types induced by application of GUT-108 in germ-free Il10⁺/^{GFP} mice. Colonic lamina propria cell were isolated from mice that had received gavage with either PBS or GUT-108 and quantified by flow cytometry after treatment with PMA/Ionomycin re-stimulation, and surface/intracellular staining. GUT-108 refers to mice that two weeks after inoculation with human stool were treated with GUT-108 in a therapeutic protocol; PBS refers to mice that two weeks after inoculation with human stool received PBS as a placebo control.

FIG. 5A is a graph showing the differences in the percentages of IL10+ CD4+ T cells induced by application of GUT-108 compared to induction by application of PBS buffer. Colonic lamina propria cells were isolated from mice that had received gavage with PBS or GUT-108 followed by PMA/Ionomycin re-stimulation, and surface/intracellular staining. Individual mouse results are represented by circles. GUT-108 refers to Il10⁺/^{eGFP} mice treated with GUT-108; PBS refers to Il10⁺/^{eGFP} mice treated with PBS buffer. Dots represent individual mouse results. Bars indicate mean. Mann-Whitney unpaired two-tailed t test. Mean+/−SE. *P<0.05; **P<0.01; ***P<0.005; ****P<0.001. N=8 for PBS, N=11 for GUT-108.

FIG. 5B is a graph showing the differences in the percentages of IL10+ B cells induced by application of GUT-108 compared to induction by application of PBS buffer as described in FIG. 5A.

FIG. 5C is a graph showing the differences in the percentages of IL10+ dendritic cells (DC) induced by application of GUT-108 compared to induction by application of PBS buffer as described in FIG. 5A.

FIG. 5D is a graph showing the differences in the percentages of IL10+ macrophages (Mf) induced by application of GUT-108 compared to induction by application of PBS buffer as described in FIG. 5A.

FIG. 5E is a graph showing the differences in the percentages of IL10+ RoRγT⁺ FoxP3⁺ CD4+ Treg cells induced by application of GUT-108 compared to induction by application of PBS buffer as described in FIG. 5A.

FIG. 5F is a graph showing the differences in the percentages of IL10+ FoxP3⁺ CD4+ Treg cells induced by application of GUT-108 compared to induction by application of PBS buffer as described in FIG. 5A.

FIG. 5G is a graph showing the differences in the peak surfaces, representative for the number of cells, obtained after fluorescence assisted cell sorting of IL10+ RoRγT⁺ FoxP3⁺ CD4+ Treg cells from a mouse induced by application of GUT-108 compared to induction by application of PBS buffer as described in FIG. 5A. The results from FIG. 5G are reported in FIG. 5E.

FIG. 5H is a graph showing the differences in the percentages of IL-10⁺ FoxP3^{neg} CD4+ Treg cells induced by application of GUT-108 compared to induction by application of PBS buffer as described in FIG. 5A.

FIG. 6 shows bacterial species whose abundance significantly changed in the gut microbiome of Il10^{-/-} mice inoculated with human fecal material to induce colitis plus therapeutic application of GUT-108 compared to control mice inoculated with human fecal material plus PBS. Two weeks before gavage with GUT-108 or PBS buffer, experimental colitis was induced by inoculating the mice with a human stool previously verified to induce aggressive colitis in gnotobiotic Il10^{-/-} mice. Differences in relative species abundance between the two treatments is expressed on a Log 10 scale. Strains with decreased or increased relative abundance are indicated in grey. GUT-108 strains are indicated in black. *p<0.05 value using the two-sided Benjamini Hochberg corrected Wilcoxon test.

FIG. 7 is a graph showing serial Lipocalin-2 levels as indication of gut inflammation at weeks 2 (W2), 3 (W3), and 4 (W4) after inoculation with human stool to induce colitis in an ex-germ-free Il10^{-/-} mouse model of IBD. Lipocalin 2 levels were determined by ELISA. “Hu+GUT-108” refers to mice that two weeks after inoculation with human stool (W2) were treated with GUT-108 in a therapeutic protocol; “Hu+PBS” refers to mice that two weeks after inoculation with human stool (W2) received PBS as a placebo control. Dots indicate individual mice data. Bar indicates mean. Mann-Whitney unpaired two-tailed t test. Mean±SE, *P<0.05, ***P<0.001, NS indicates not significant. N=11/group.

FIG. 8A is an image showing stool consistency as indication of gut inflammation after inoculation with human stool to induce colitis in an ex-germ-free Il10^{-/-} mouse model of IBD. Hu+GUT-108 refers to mice that two weeks after inoculation with human stool were treated with GUT-108 in a therapeutic protocol; Hu+PBS refers to mice that two weeks after inoculation with human stool received PBS as a placebo control. Stool consistency was scored according to Gut Pathogens 2011; 3:16, as follows: Score 0: normal; Score 1: loose stool; Score 2: loose/some diarrhea; Score 3: diarrhea; and Score 4: severe watery diarrhea.

FIG. 8B is a graph showing stool consistency as indication of gut inflammation in the mouse model of IBD scored as shown in FIG. 8A at weeks 2 (W2), 3 (W3), and 4 (W4) after inoculation with human stool to induce colitis. Mean+/−SE. Mann-Whitney unpaired two-tailed t test. *P<0.05, NS indicates not significant. Stool consistency scores for both treatments were normal (scored as “0”) for week 2.

FIG. 9A is a representative distal colonic photomicrographs of H&E-stained tissue showing the severity of gut inflammation of the colonic tissues in an ex-germ-free Il10^{-/-} mouse model of IBD, in a mouse that received PBS as a placebo two weeks after inoculation with human stool to induce colitis. Bar in upper right corner indicates 100 μm. The experiment was repeated independently with similar results for each animal in the PBS (N=11) group.

FIG. 9B is a representative distal colonic photomicrographs of H&E-stained tissue showing the effect of GUT-108 in reducing the severity of inflammation of the colonic tissues in an ex-germ-free Il10^{-/-} mouse model of IBD, in a mouse treated with GUT-108 two weeks after inoculation with human stool to induce colitis. Bar in upper right corner indicates 100 μm. The experiment was repeated independently with similar results for each animal in the GUT-108 (N=11) group.

FIG. 10 is a graph showing the effect of therapeutic application of GUT-108 on the severity of gut inflammation in Il10^{-/-} mice. Blinded histologic scoring was used to assess the level of inflammation for different parts of the intestine, including the distal ileum (Ile), cecum (Ce), proximal- (Pc) and distal-(Dc) colon, rectum (Re), and combined (Ce+Pc+Dc+Re). Hu+GUT-108 refers to mice that two weeks after inoculation with human stool were treated with GUT-108 in a therapeutic protocol; Hu+PBS refers to mice that two weeks after inoculation with human stool received PBS as a placebo control. Dot represents individual mouse result. Bar indicates mean. Mann-Whitney unpaired two-tailed t test. *P<***P<0.001.

FIG. 11A is a graph showing levels of short chain fatty acid propionic acid in the Il10^{-/-} knock-out mice. Hu+GUT-108 refers to Il10^{-/-} mice that two weeks after inoculation with human stool were treated with GUT-108 in a therapeutic protocol; Hu+PBS refers to Il10^{-/-} mice that two weeks after inoculation with human stool received PBS as a placebo control. Two-way ANOVA and adjusted P values were calculated by the multiple comparisons test. Mean+/−SE. *P<0.05, NS indicates not significant.

FIG. 11B is a graph showing levels of short chain fatty acid butyric acid in the Il10^{-/-} knock-out mice. Hu+GUT-108 refers to Il10^{-/-} mice that two weeks after inoculation with human stool were treated with GUT-108 in a therapeutic protocol; Hu+PBS refers to Il10^{-/-} mice that two weeks after inoculation with human stool received PBS as a placebo control. Two-way ANOVA and adjusted P values were calculated by the multiple comparisons test. Mean+/−SE. NS indicates not significant.

FIG. 12A is a graph showing levels of indole in the Il10^{-/-} knock-out mice humanized with a fecal transplant. Hu+GUT-108 refers to mice that two weeks after inoculation with human stool were treated with GUT-108 in a therapeutic protocol; Hu+PBS refers to mice that two weeks after inoculation with human stool received PBS as a placebo control. Two-way ANOVA and adjusted P values were calculated by the multiple comparisons test. Mean+/−SE. **P<0.01, NS indicates not significant.

FIG. 12B is a graph showing levels of indole derivative indole propionate (IPA) in the Il10^{-/-} knock-out mice humanized with a fecal transplant. Hu+GUT-108 refers to mice that two weeks after inoculation with human stool were treated with GUT-108 in a therapeutic protocol; Hu+PBS refers to mice that two weeks after inoculation with human stool received PBS as a placebo control. Two-way ANOVA and adjusted P values were calculated by the multiple comparisons test. Mean+/−SE. **P<NS indicates not significant.

FIG. 12C is a graph showing levels of indole derivative indole acetate (IAA) in the Il10^{-/-} knock-out mice humanized with a fecal transplant. Hu+GUT-108 refers to mice that two weeks after inoculation with human stool were treated with GUT-108 in a therapeutic protocol; Hu+PBS refers to mice that two weeks after inoculation with human stool received PBS as a placebo control. Two-way ANOVA and adjusted P values were calculated by the multiple comparisons test. Mean+/−SE. **P<NS indicates not significant.

FIG. 13A is a graph showing levels of secondary bile acid deoxycholic acid (DCA) in the IL10 knock-out mice humanized with a fecal transplant. Hu+GUT-108 refers to mice that two weeks after inoculation with human stool were treated with GUT-108 in a therapeutic protocol; Hu+PBS refers to mice that two weeks after inoculation with human stool received PBS as a placebo control. Two-way ANOVA and adjusted P values were calculated by the multiple comparisons test. Mean+/−SE. ***P<0.001, NS indicates not significant.

FIG. 13B is a graph showing levels of secondary bile acid lithocholic acid (LCA) in the IL10 knock-out mice humanized with a fecal transplant. Hu+GUT-108 refers to mice that two weeks after inoculation with human stool were treated with GUT-108 in a therapeutic protocol; Hu+PBS refers to mice that two weeks after inoculation with human stool received PBS as a placebo control. Two-way ANOVA and adjusted P values were calculated by the multiple comparisons test. Mean+/−SE. ***P<0.001, NS indicates not significant.

FIG. 14A is a graph showing synthesis of IL-12p40 by gut cell cultures of Il10^{-/-} knock-out mice humanized with a fecal transplant. IL-12p40 synthesis was determined by ELISA after 20 hours. GUT-108 refers to mice that two weeks after inoculation with human stool were treated with GUT-108 in a therapeutic protocol; PBS refers to mice that two weeks after inoculation with human stool received PBS as a placebo control. Dots indicate individual mice data. Mean+/−SE. Unpaired two-tailed t test. **P<0.01. N=11/group.

FIG. 14B is a graph showing synthesis of IFN-γ by gut cell cultures of Il10^{-/-} knock-out mice humanized with a fecal transplant. IFN-γ synthesis was determined by ELISA after 20 hours. Dots indicate individual mice data. Mean+/−SE. Unpaired two-tailed t test. N=11/group.

FIG. 15A is a graph showing the effect of GUT-108 on the level of expression of the biosynthesis genes for the pro-inflammatory cytokine IL-1β in gut tissue of Il10^{-/-} knock-out mice humanized with a fecal transplant as measured by Q-RP-PCR. Colonic lamina propria cell were isolated from mice that had either been inoculated with human donor stool or human donor stool plus GUT-108 in a therapeutic protocol, and total messenger RNA (mRNA) was isolated. Subsequently, mRNA was converted into cDNA, which was subsequently used in a Quantitative PCR (RT-Q-PCR) protocol to estimate levels of gene expression using gene specific primers. Hu+GUT-108 refers to mice that two weeks after inoculation with human stool were treated with GUT-108 in a therapeutic protocol; Hu+PBS refers to mice that two weeks after inoculation with human stool received PBS as a placebo control. Differences in gene expression were presented as fold change (FC) of gene expression in the Hu+GUT-108 group compared to the Hu+PBS placebo control group. Bar indicates mean+/−SE. Two-way ANOVA and adjusted P value were calculated by the multiple comparisons test. *P<0.05. N=11/group.

FIG. 15B is a graph showing the effect of GUT-108 on the level of expression of the biosynthesis genes for the pro-inflammatory cytokine IL-12b in gut tissue of Il10^{-/-} knock-out mice humanized with a fecal transplant as described in FIG. 15A.

FIG. 15C is a graph showing the effect of GUT-108 on the level of expression of the biosynthesis genes for the pro-inflammatory cytokine IL-17α in gut tissue of Il10^{-/-} knock-out mice humanized with a fecal transplant as described in FIG. 15A.

FIG. 15D is a graph showing the effect of GUT-108 on the level of expression of the biosynthesis genes for the pro-inflammatory cytokine IFNγ in gut tissue of Il10^{-/-} knock-out mice humanized with a fecal transplant as described in FIG. 15A.

FIG. 15E is a graph showing the effect of GUT-108 on the level of expression of the biosynthesis genes for the pro-inflammatory cytokine IL-13 in gut tissue of Il10^{-/-} knock-out mice humanized with a fecal transplant as described in FIG. 15A.

FIG. 15F is a graph showing the effect of GUT-108 on the level of expression of the biosynthesis genes for the pro-inflammatory cytokine TNFα in gut tissue of Il10^{-/-} knock-out mice humanized with a fecal transplant as described in FIG. 15A.

FIG. 15G is a graph showing the effect of GUT-108 on the level of expression of the biosynthesis genes for the homeostatic cytokine IL-15 in gut tissue of Il10^{-/-} knock-out mice humanized with a fecal transplant as described in FIG. 15A.

FIG. 16A is a graph showing the effect of GUT-108 on the level of expression of the aryl hydrocarbon receptor (Ahr) gene in gut tissue of Il10^{-/-} knock-out mice humanized with a fecal transplant as measured by Q-RP-PCR. Colonic lamina propria cell were isolated from Il10^{-/-} mice that had either been inoculated with human donor stool or human donor stool plus GUT-108 in a therapeutic protocol, and total messenger RNA (mRNA) was isolated. Subsequently, mRNA was converted into cDNA, which was subsequently used in a Quantitative PCR (RT-Q-PCR) protocol to estimate levels of gene expression using gene specific primers. Expression levels were determined by RT-Q-PCR and presented as fold change (FC) of expression after therapeutic application of GUT-108 compared to expression after PBS treatment. Hu+GUT-108 refers to mice that two weeks after inoculation with human stool were treated with GUT-108 in a therapeutic protocol; Hu+PBS refers to mice that two weeks after inoculation with human stool received PBS as a placebo control. Bar indicates mean+/−SEM. Two-way ANOVA and adjusted P value were calculated by the multiple comparisons test. *P<N=11/group.

FIG. 16B is a graph showing the effect of GUT-108 on the level of expression of the aryl hydrocarbon receptor repressor (AhrR) gene in gut tissue of Il10^{-/-} knock-out mice humanized with a fecal transplant as described in FIG. 16A.

FIG. 16C is a graph showing the effect of GUT-108 on the level of expression of the biosynthesis gene for the Cytochrome P450 Family 1 Subfamily A Member 1 (Cyp1A1) gene in gut tissue of Il10^{-/-} knock-out mice humanized with a fecal transplant as described in FIG. 16A.

FIG. 16D is a graph showing the effect of GUT-108 on the level of expression of the defensin DefCR1 gene in gut tissue of Il10^{-/-} knock-out mice humanized with a fecal transplant as described in FIG. 16A.

FIG. 16E is a graph showing the effect of GUT-108 on the level of expression of the defensin DefA gene in gut tissue of Il10^{-/-} knock-out mice humanized with a fecal transplant as described in FIG. 16A.

FIG. 16F is a graph showing the effect of GUT-108 on the level of expression of the aldehyde dehydrogenase Aldh1A1 gene in gut tissue of Il10^{-/-} knock-out mice humanized with a fecal transplant as described in FIG. 16A.

FIG. 16G is a graph showing the effect of GUT-108 on the level of expression of the aldehyde dehydrogenase Aldh1A2 gene in gut tissue of Il10^{-/-} knock-out mice humanized with a fecal transplant as described in FIG. 16A.

FIG. 17A is a graph showing the effect of GUT-108 on the level of pro-inflammatory IFN-γ+ synthesizing CD4+ T cells in gut tissue of Il10^{-/-} knock-out mice humanized with a fecal transplant. Colonic lamina propria cell were isolated from mice that had either been inoculated with human donor stool or human donor stool plus GUT-108 in a therapeutic protocol and quantified by flow cytometry after treatment with PMA/Ionomycin re-stimulation, and surface/intracellular staining. Each dot represents an individual mouse result. GUT-108 refers to mice that two weeks after inoculation with human stool were treated with GUT-108 in a therapeutic protocol; PBS refers to mice that two weeks after inoculation with human stool received PBS as a placebo control. Bar indicates mean. Mann-Whitney unpaired two tailed t test. *P<0.05. % in total CD4⁺ T cells (singlet-Live/Dead^{neg}CD45⁺CD3⁺TCRβ⁺CD4⁺).

FIG. 17B is a graph showing the effect of GUT-108 on the level of pro-inflammatory IFN-γ+ IL-17α+ synthesizing CD4+ T cells in gut tissue of Il10^{-/-} knock-out mice humanized with a fecal transplant as described in FIG. 17A.

FIG. 17C is a graph showing the effect of GUT-108 on the level of pro-inflammatory IL-17α+ synthesizing CD4+ T cells in gut tissue of Il10^{-/-} knock-out mice humanized with a fecal transplant as described in FIG. 17A.

FIG. 18A is a graph showing the effect of GUT-108 on the level of regulatory FOXP3+ T cells in colonic tissue of Il10^{-/-} knock-out mice humanized with a fecal transplant. Colonic lamina propria cells were isolated from mice that had either been inoculated with human donor stool or human donor stool plus GUT-108 in a therapeutic protocol and quantified by flow cytometry after treatment with PMA/lonomycin re-stimulation, and surface/intracellular staining. Each dot represents an individual mouse result. GUT-108 refers to mice that two weeks after inoculation with human stool were treated with GUT-108 in a therapeutic protocol; PBS refers to mice that two weeks after inoculation with human stool received PBS as a placebo control. Bar indicates mean. Mann-Whitney unpaired two tailed t test. *P<0.05. % in total CD4⁺ T cells (singlet-Live/Dead^{neg}CD45⁺CD3⁺TCRβ⁺CD4⁺).

FIG. 18B is a graph showing the effect of GUT-108 on the level of regulatory FOXP3+RORγt+CD4+ T cells in colonic tissue of Il10^{-/-} knock-out mice humanized with a fecal transplant as described in FIG. 18A.

### DETAILED DESCRIPTION

The presently disclosed subject matter now will be described more fully hereinafter. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the descriptions provided herein. Therefore, it is to be understood that the presently disclosed subject matter is not to be limited to the specific embodiments disclosed and that modifications and other embodiments are intended to be included within the scope of the appended claims.

Following long-standing patent law convention, the terms “a,” “an,” and “the” refer to “one or more” when used in this application, including the claims. Thus, for example, reference to “a subject” includes a plurality of subjects, unless the context clearly is to the contrary, and so forth.

Throughout this specification and the claims, the terms “comprise,” “comprises,” and “comprising” are used in a non-exclusive sense, except where the context requires otherwise. Likewise, the terms “having” and “including” and their grammatical variants are intended to be non-limiting, such that recitation of items in a list is not to the exclusion of other like items that can be substituted or added to the listed items.

For the purposes of this specification and claims, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range. In addition, as used herein, the term “about”, when referring to a value can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed compositions and methods. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

Throughout this specification and the claims, the term “subject” includes humans and animals and can be used interchangeably with the term “human” and the term “patient”.

As used herein, the phrase “a biologically pure culture of” includes one or a combination of spores and vegetative cells of the biologically pure fermentation culture of the bacterial strain. By “biologically pure” is meant essentially biologically pure as it is understood in the art. In addition, the phrases “biologically pure culture of a bacterium” and “bacterial strain” and “microbial strain” and “microbe” are herein used interchangeably for the purposes of the specification and claims and include a mutant of the microbial or bacterial strain having all the identifying characteristics thereof.

As used herein for the purposes of the specification and claims, the phrase “a method of treating inflammation in a subject” includes a method of treating inflammation in subjects at risk for an inflammatory disorder (e.g., preventing inflammation) and a method of treating inflammation in subjects having an inflammatory disorder.

As used herein for the purposes of the specification and claims, the phrase “having genetic material encoding for” includes genetic material that is indicated as a putative gene based on bioinformatic methodologies.

In one embodiment, a therapeutic microbial consortium is provided that is designed to provide key functionalities that are lacking or underrepresented in the dysbiotic gut microbiome of subjects having an inflammatory disorder. These functionalities can address both inflammation and infection control. The rationally designed 11-strain consortium provided herein is referred to as GUT-108. The advantageous features of GUT-108 include that its members create a network of metabolic dependencies designed to promote engraftment and stability of the consortium in the hostile dysbiotic environment of intestinal inflammation.

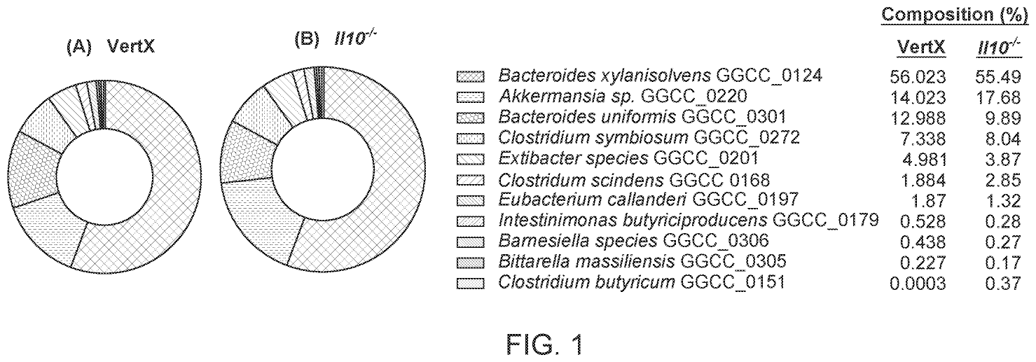

GUT 108, whose design using newly isolated and previously unidentified strains is presented in EXAMPLE 1, has been tested in a clinically relevant mouse model where chronic immune-mediated colitis was induced by inoculating the mice with a human stool as described in EXAMPLE 2 herein and illustrated in FIGS. 1-18. In addition to reducing the induced inflammation in the cecum, GUT-108 had a surprisingly beneficial effect on the composition of the mouse gut microbiome. Specifically, a healthier gut microbiome was restored in the GUT-108 treated mice, and the healthier microbiome had an abundance of beneficial Clostridium strains that are not present in GUT-108. In addition, the abundance of pathogenic Enterobacteriaceae bacteria introduced into the mouse gut to induce the disease state, decreased from 5% to below 0.5% after treatment with GUT-108. Further, an eight-fold decrease in the levels of the pathogenic Clostridium perfringens was also observed after treatment with GUT-108. Without being limited to any one mechanism of action, the mouse model results described herein indicate that the GUT-108 strains may be exerting these protective effects by producing metabolites that control pathogenic bacteria and provide an environment supportive of beneficial Clostridium species, promote mucosal healing and regulatory immune responses, inhibit inflammatory innate, Th1, and Th17 cytokines, and induce lamina propria regulatory T cells, B cells, and dendritic cells. These results indicate that GUT-108 can be useful in a method of treating infection and/or inflammation in a subject.

The microbial therapeutic consortium provided herein includes bacterial strains having the following functionalities that, without being limited to any one mechanism of action, are: butyrate synthesis and propionate synthesis for stimulation of Tregs, modulation of the immune response (e.g. affecting synthesis of the interleukins IL-1, IL-6, IL-12, IL13 and IL-17, and IFNγ and TNFα to lower the (pre) inflammatory condition; or higher synthesis levels of anti-inflammatory IL-10 and the homeostatic cytokine IL-15), and modulation of bowel movements (clinically relevant at a minimum to IBD patients that experience diarrhea); synthesis of indole and its derivatives to tighten the epithelial cell junctions and activate the Ahr pathway, which is critical to avoid excessive pro-inflammatory cytokine expression, septic shock, and to protect the mucosa during inflammation; GABA synthesis for further inhibition of (autoimmune) inflammation; functions involved in the deconjugation and conversion of bile salts into secondary bile acids which have therapeutic properties to restore intestinal epithelial stem cell function, increase colonic RORγ+ Treg cell counts that ameliorate host susceptibility to colitis, stimulate differentiation to Tregs and inhibit Th17 cells, and provide antagonistic activities against pathogens; siderophore synthesis and/or uptake of heterologous siderophore to efficiently compete with (opportunistic) pathogens for the essential nutrient iron; and synthesis of antagonistic molecules, including bacteriocins.

Inoculation with the 11 strain GUT-108 consortium increased gene expression levels including the defensins DefCR1 and DefA, whose altered production is suggested to be integrally involved in IBD pathogenesis; and the retinoic acid pathway that involves the aldehyde dehydrogenases Aldh1A1 and Aldh1A2, and which is critical in regulating Wnt/β-catenin signaling. Studies indicate that reduced activity of the retinoic acid pathway relate to tumor development and cellular migration. Several cellular pathways, including the Wnt/β-catenin signaling pathway, are related to cancer metastasis, and many reports have suggested that exaggerated Wnt signaling can lead to cancer initiation and progression in a wide range of human tissues, including colon cancer. Therefore, in one embodiment, administration of GUT-108 can lower the risk of colorectal cancer in IBD patients.

In the rational design of a microbial therapeutic consortium as described herein, the key functionalities described above are distributed over the various members of the consortium, redundancies in functionalities are included, and the consortium includes bacteria other than those belonging to the spore forming members of Clostridium clusters IV and XIVa, in order to cover several key functionalities including the synthesis of propionate and indole that members of these Clostridium clusters are largely lacking (see EXAMPLE 1). In addition to the key functionalities, complementary auxotrophies are also distributed over the various members of the consortium, which provides a network of metabolic inter-dependencies between the strains. As with the key functionalities, redundancy has also been built into the auxotrophies for the strains in the consortium. In one embodiment, the bacterial consortia of the present disclosure include complementary auxotrophies to provide metabolic interdependency.

A consortium of 11 strains containing the key functionalities listed in the previous paragraph and complementary auxotrophies for metabolic interdependency is provided and referred to as GUT-108. The strains and key functionalities are shown in Table 1 and the auxotrophies providing the network of metabolic inter-dependencies are shown in Table 2. The designated species of each of the newly isolated strains is the closest relationship based on sequence homology to 16S rRNA gene sequence. Specifically, Akkermansia species—like strain GGCC_0220 (SEQ ID NO: 1), Bacteroides uniformis—like strain GGCC_0301 (SEQ ID NO: 2), Bacteroides xylanisolvens—like strain GGCC_0124 (SEQ ID NO: 3), Barnesiella species—like strain GGCC_0306 (SEQ ID NO: 4), Bitterella massiliensis—like strain GGCC_0305 (SEQ ID NO: 5), Clostridium butyricum—like strain GGCC_0151 (SEQ ID NO: 6), Clostridium scindens— like strain GGCC_0168 (SEQ ID NO: 7), Clostridium symbiosum—like strain GGCC_0272 (SEQ ID NO: 8), Eubacterium callanderi—like strain GGCC_0197 (SEQ ID NO: 9), Extibacter species—like strain GGCC_0201 (SEQ ID NO: 10), and Intestinimonas butyriciproducens—like strain GGCC_0179 (SEQ ID NO: 11).

The rational design of GUT-108 included building in a high level of metabolic interdependency between the individual member strains to promote stability of the consortium in the hostile dysbiotic environment of intestinal inflammation. To achieve this high level of metabolic interdependency, an auxotrophy analysis was performed as described in EXAMPLE 1. The auxotrophy profile of GUT-108 shown in Table 2 reveals the substantial differences in auxotrophy between the individual strains.

In one embodiment, compositions including the GUT-108 bacterial strains disclosed herein are provided for the treatment of subjects at risk for or having an inflammatory disorder.

In one embodiment, a method is provided that includes administering a composition of the present disclosure to a subject to prevent or to decrease inflammation. The inflammation can be a result of one or more disorders including Inflammatory Bowel Diseases (IBD) like ulcerative colitis and Crohn's disease, Pouchitis, Irritable Bowel Syndrome (IBS), graft versus host disease, Parkinson's disease, ALS, multiple sclerosis, systemic lupus erythematosus, Ankylosing Spondylitis, asthma, food allergies, fatty liver, Primary Sclerosing Cholangitis as comorbidity for IBD, hepatic encephalopathy, type-2 diabetes, metabolic syndrome and obesity, Plaque Psoriasis, Psoriatic Arthritis, and aging related conditions of chronic inflammation.

The design process purposely introduced redundancies in functionalities to increase the chances of establishment of the consortium or a subset of strains thereof under a broad range of conditions, thus addressing different degrees of gut microbiome dysbiosis. Based on classification (EzBioCloud database (https://help.ezbiocloud .net/taxonomy-of-clostridium-cluster-xiva-iv/) and Bacterio.net (http://www.bacterio.net/-classifphyla.html)) for the assignment of species belonging to the families of the Lachnospiraceae and the Ruminococcaceae to specific Clostridium clusters, only Clostridium symbiosum—like strain GGCC_0272 (spore forming) and Bitterella massiliensis—like strain GGCC_0305 (non-spore forming; Durand et al, 2017) are considered to belong to the Clostridium cluster XIVa. Based on annotation and modeling, a putative Yersiniabactin synthesis operon is identified for Clostridium symbiosum—like strain GGCC_0272, and this strain is provided for having the functionality of producing its own siderophore.

Based on annotation and modeling, the function of uptake of the heterologously produced siderophore aerobactin is identified for Bacteroides uniformis—like strain GGCC_0301, Bacteroides xylanisolvens—like strain GGCC_0124, and Barnesiella species—like strain GGCC_0306, and uptake of the heterologously produced siderophore enterobactin is identified for Barnesiella species—like strain GGCC_0306, Bitterella massiliensis—like strain GGCC_0305, Clostridium butyricum—like strain GGCC_0151, Clostridium symbiosum—like strain GGCC_0272, Eubacterium callanderi—like strain GGCC_0197, Extibacter species—like strain GGCC_0201, and Intestinimonas butyriciproducens—like strain GGCC_0179. These strains are provided for having the functionality of uptake of heterologous siderophores. The presence of heterologous siderophore uptake systems can allow for strains to compete against opportunistic pathogenic bacteria (including enterohemorrhagic, enteroaggregative, and adherent invasive Escherichia coli and Klebsiella species) that can thrive in the dysbiotic gut environment associated with IBD and other conditions of chronic inflammation.

Based on annotation and modeling, strains of GUT-108 for inflammation control are identified as having a range of activities to deconjugate and subsequently modify bile salts into secondary bile acids (see Table 1). Clostridium scindens species—like strain GGCC_0168 was identified as having putative bile acid 7-alpha-dehydratase activity as part of its bai operon. Unexpectedly, strains Eubacterium callanderi—like strain GGCC_0197 and Extibacter species—like strain GGCC_0201 are also identified as having putative bile acid 7-alpha-dehydratase activity. This activity has not been previously reported for strains of Eubacterium callanderi or Extibacter species. For Extibacter species—like strain GGCC_0201, the putative 7-alpha-dehydratase is part of a bai operon with additional genes putatively encoding a bile acid-coenzyme A ligase, a NADH oxidase, a L-carnitine dehydratase/bile acid-inducible protein F, a bile acid transporter, and a 7-beta-dihydroxy-3-oxo-5-beta-cholanoyl-CoA 4-oxidoreductase. The bile acid 7-alpha-dehydratase activity catalyzes a critical step in the synthesis of lithocholic acid and deoxycholic acid. Thus, deoxycholic acid synthesis may play an important role in the control of pathogenic Clostridium bacteria, including Clostridium perfringens and Clostridium difficile that can thrive in a dysbiotic gut environment associated with conditions of chronic and severe inflammation.

Based on annotation and modeling, strains of GUT-108 are identified as having key functionalities for bacteriocin and lantibiotic synthesis and resistance as shown in Table 1.

Based on annotation and modeling, Akkermansia species—like strain GGCC_0220, Bacteroides uniformis—like strain GGCC_0301, and Bacteroides xylanisolvens—like strain GGCC_0124 are identified as having functionality for synthesis of indole (i.e., a putative Tryptophanase (EC 4.1.99.1) gene that catalyzes the breakdown of tryptophan into indole, pyruvate and NH₃).

Based on annotation and modeling, Akkermansia species—like strain GGCC_0220, Bacteroides uniformis—like strain GGCC_0301, Bacteroides xylanisolvens—like strain GGCC_0124, and Barnesiella species—like strain GGCC_0306 are identified as having functionality for synthesis of short chain fatty acid, propionate (see Table 1). Clostridium butyricum—like strain GGCC_0151, Clostridium symbiosum—like strain GGCC_0272, and Eubacterium callanderi—like strain GGCC_0197 are identified as having functionality for synthesis of short chain fatty acid, butyrate (see Table 1).

Based on annotation and modeling, Akkermansia species—like strain GGCC_0220, Bacteroides uniformis—like strain GGCC_0301, Bacteroides xylanisolvens—like strain GGCC_0124, Barnesiella species—like strain GGCC_0306, and Eubacterium callanderi—like strain GGCC_0197 are identified as having a functionality for catalyzing the breakdown of glutamate into GABA and CO₂, (i.e., synthesis of GABA) (see Table 1).

TABLE 1
Summary of key functionalities identified for the members of the eleven-strain rationally designed consortium
for IBD control, referred to as the GUT-108 consortium. CGH: choloylglycine hydrolase; LCD: L-cantinine
hydratase; 3-oxo-5α: 3-oxo-5-alpha-steroid-4-dehydrogenase; 3α-HSD: 3α-hydroxy steroid dehydrogenase;
3β-HSD: 3β-hydroxy steroid dehydrogenase; 7α-HSD: 7α-hydroxy steroid dehydrogenase; 3α-CHD:
3α-hydroxycholate dehydrogenase; 3α-DH: 3α-hydroxy bile acid-CoA-ester-3 dehydrogenase; 7α-DH:
7α-dehydratase; 7β-DH: 7β-dehydratase; SBS: sodium-bile acid symporter system. The EzBioCloud database
(https://help.ezbiocloud.net/taxonomy-of-clostridium-cluster-xiva-iv/) and Bacterio.net (http://www.bacterio.net/-
classifphyla.html) was used for the assignment of species belonging to the families of the Lachnospiraceae
and the Ruminococcaceae to specific Clostridium clusters.

| | | | | | | Clostridium | |
|---|---|---|---|---|---|---|---|
| Strain ID | Butyrate | Propionate | GABA | Indole | Bile Acid | cluster | antiSMASH and Comments |
| Akkermansia | | + | + | + | SBS | N.A. | DJGMFOLP_00714: |
| species | | | | | | | Dihydroneopterin aldolase, |
| GGCC_0220 | | | | | | | with possible role in the |
| | | | | | | | biosynthesis of the |
| | | | | | | | neurotransmitter serotonin (5- |
| | | | | | | | hydroxytryptamine, 5-HT), |
| | | | | | | | melatonin, dopamine, |
| | | | | | | | norepinephrine (noradrenaline), |
| | | | | | | | epinephrine (adrenaline); |
| | | | | | | | DJGMFOLP_00716: phytoene |
| | | | | | | | synthase; phytoene is a 40- |
| | | | | | | | carbon intermediate in the |
| | | | | | | | biosynthesis of carotenoids; |
| | | | | | | | DJGMFOLP_01670: Squalene |
| | | | | | | | synthase |
| Bacteroides | | + | + | + | CGH | N.A. | GIHOKGLK_00696: resorcinol |
| uniformis | | | | | 3-oxo-5-α | | biosynthesis like - antiseptic |
| GGCC_0301 | | | | | SBS | | and disinfectant in topical |
| | | | | | | | pharmaceutical products in the |
| | | | | | | | treatment of skin disorders and |
| | | | | | | | infections |
| Bacteroides | | + | + | + | 7-α-HSD | N.A. | Propionate and succinate |
| xylanisolvens | | | | | CGH | | producer; |
| GGCC_0124 | | | | | 3-oxo-5-α | | BLEPDCHD_01336: resorcinol |
| | | | | | | | biosynthesis like - antiseptic |
| | | | | | | | and disinfectant in topical |
| | | | | | | | pharmaceutical products in the |
| | | | | | | | treatment of skin disorders and |
| | | | | | | | infections; |
| | | | | | | | BLEPDCHD_01621: NRPS - |
| | | | | | | | polyketide synthase; |
| | | | | | | | BLEPDCHD_03897-90307: Aryl |
| | | | | | | | polyene biosynthesis for |
| | | | | | | | antioxidative carotenoid-like |
| | | | | | | | compound |
| Barnesiella | | + | + | | CGH | N.A. | Based on 16S rRNA gene |
| species | | | | | 3-oxo-5-α | | sequence, closest related to |
| GGCC_0306 | | | | | | | Barnesiella viscericola strain C46 |
| | | | | | | | (93% identity) |
| | | | | | | | No antiSMASH results |
| Bitterella | | | | | 3-α-HSD | XIVa | Non-spore forming (Durand et |
| massiliensis | | | | | 3-β-HSD | | al, 2017); |
| GGCC_0305 | | | | | | | Key for bile acid conversion via |
| | | | | | | | 3α-hydroxy steroid |
| | | | | | | | dehydrogenase and 3β-hydroxy |
| | | | | | | | steroid dehydrogenase |
| | | | | | | | activities; |
| | | | | | | | KIFOIBJG_00615-00616: |
| | | | | | | | Phenazine biosynthesis phzAB; |
| | | | | | | | KIFOIBJG_02615: putative beta- |
| | | | | | | | lactone synthesis |
| Clostridium | + | | | | 7-α-HSD | I | JOINCCCK_01237-01248: |
| butyricum | | | | | CGH | | NRPS - polyketide synthase |
| GGCC_0151 | | | | | Taurine | | closest related to Circularin A |
| | | | | | uptake | | (antimicrobial); |
| | | | | | | | JOINCCCK_01886-01892: |
| | | | | | | | Lassopeptide - a wide range of |
| | | | | | | | interesting biological activities |
| | | | | | | | are known for these |
| | | | | | | | compounds, including |
| | | | | | | | antimicrobial, enzyme |
| | | | | | | | inhibitory, and receptor |
| | | | | | | | antagonistic activities. |
| Clostridium | | | | | 7-α-DH, | Clostr. G21 | Key for bile acid conversion via |
| scindens | | | | | 3-α-DH, | | 7α-dehydratase activity and 3α- |
| GGCC_0168 | | | | | 7-α-HSD | | hydroxy bile acid-CoA-ester-3 |
| | | | | | CGH | | dehydrogenase; |
| | | | | | LCD | | OPHKCAJD_00380-381: |
| | | | | | | | Sactipeptide synthesis module; |
| | | | | | | | These peptides (e.g. subtilosin |
| | | | | | | | A) can have antimicrobial |
| | | | | | | | activity; |
| | | | | | | | OPHKCAJD_01868-01872: |
| | | | | | | | NRPS operon; |
| | | | | | | | OPHKCAJD_02406: Bacteriocin |
| | | | | | | | (Lactococcin 972 - like) |
| Clostridium | + | | | | 7-α-HSD, | XIVa; | FHPFMLLL_00026-00029: |
| symbiosum | | | | | CGH | Clostr. G24 | Yersiniabactin synthesis operon |
| GGCC_0272 | | | | | LCD | | (siderophore); |
| | | | | | Taurine | | FHPFMLLL_04833-04834: |
| | | | | | uptake | | Sactipeptide synthesis module; |
| | | | | | | | These peptides (e.g. subtilosin |
| | | | | | | | A) can have antimicrobial |
| | | | | | | | activity. |
| Eubacterium | + | | + | | 7-α-DH | XV | FDEBBACK_00621-00625: |
| callanderi | | | | | 7-α-HSD | | Linocin M18 like bacteriocin |
| GGCC_0197 | | | | | CGH | | synthesis and resistance operon; |
| | | | | | SBS | | FDEBBACK_01439-01440: |
| | | | | | | | Sactipeptide synthesis module; |
| | | | | | | | These peptides (e.g. subtilosin |
| | | | | | | | A) can have antimicrobial |
| | | | | | | | activity; |
| | | | | | | | FDEBBACK_02238-02242: |
| | | | | | | | terpene biosynthesis, putatively |
| | | | | | | | phytoene synthase. Phytoene is |
| | | | | | | | a 40-carbon intermediate in the |
| | | | | | | | biosynthesis of carotenoids; |
| | | | | | | | FDEBBACK_03367: NRPS - |
| | | | | | | | polyketide synthase; |
| | | | | | | | FDEBBACK_03748-03749: |
| | | | | | | | Maritimacin or Linocin M18 - |
| | | | | | | | like bacteriocin synthesis |
| Extibacter | | | | | 7α/β -DH | Extibacter | Based on 16S rRNA gene |
| species | | | | | LCD | | sequence, closest related to |
| GGCC_0201 | | | | | | | Extibacter muris strain 40cc-B- |
| | | | | | | | 5824-ARE (96% identity); |
| | | | | | | | Key for bile acid conversion via |
| | | | | | | | 7-α-DH activity |
| | | | | | | | BNAPOCOJ_01147, located in a |
| | | | | | | | bile acid conversion operon: |
| | | | | | | | bacteriocin; |
| | | | | | | | BNAPOCOJ_01819-01820: |
| | | | | | | | lantipeptde synthesis (LanM |
| | | | | | | | plus precursor peptide); |
| | | | | | | | BNAPOCOJ_01924-01926: |
| | | | | | | | Sactipeptide synthesis module; |
| | | | | | | | These peptides (e.g. subtilosin |
| | | | | | | | A) can have antimicrobial |
| | | | | | | | activity; |
| | | | | | | | BNAPOCOJ_02838-02839: |
| | | | | | | | lantipeptde synthesis (LanM |
| | | | | | | | plus precursor peptide) |
| Intestinimonas | + | | | | LCD | unassigned | JGHDODPO_03775: Linocin M18 |
| butyriciproducens | | | | | | | like bacteriocin like; |
| GCC_0179 | | | | | | | Putative Lassopeptide located |
| | | | | | | | on contig 49 (Pos: 2352-2468) |

TABLE 2
Summary of the auxotrophies identified in silio based on incomplete metabolic pathways for the members of the eleven-
strain rationally designed consortium for IBD control, referred to as the GUT-108 consortium. Auxotrophies were identified
for key amino acids, vitamins and co-factors. AA: auxotrophic; +: able to synthesize the compound. Amino acids
are represented by their 3-letter code. Co-factors and vitamins are represented by their 3-letter code. SAM: S-adenosyl-
methionine; Fol: folate; PAN: pantoate; Nia: niacin; Thi: thiamine; Rib: riboflavin; Cbl: cobalamin.

| | Species | Sper | Arg | Pro | Gly | Ser | Thr | Ala | Asp | Asn | Glu | Trp | Tyr | Phe |
|---|---|---|---|---|---|---|---|---|---|---|---|---|---|---|
| Bacteroides | GGCC_0124 | AA | + | + | + | + | + | + | + | + | + | + | + | + |
| xylanisolvens | | | | | | | | | | | | | | |
| Clostridium | GGCC_0151 | + | + | + | + | + | + | + | + | + | + | + | + | + |
| butyricum | | | | | | | | | | | | | | |
| Clostridium | GGCC_0168 | AA | + | + | + | + | + | + | + | + | + | AA | + | + |
| scindens | | | | | | | | | | | | | | |
| Intestinimonas | GGCC_0179 | AA | + | + | + | + | + | + | + | + | + | + | + | + |
| butyriciproducens | | | | | | | | | | | | | | |
| Eubacterium | GGCC_0197 | AA | + | + | + | + | + | + | + | + | + | + | + | + |
| callanderi | GGCC_0201 | AA | + | + | + | + | + | + | + | + | + | AA | + | + |
| Extibacter sp. | GGCC_0220 | AA | + | + | + | + | + | + | + | AA | + | + | + | + |
| Akkermansia sp. | AA | + | + | + | + | + | + | + | AA | + | + | + | + | + |
| Clostridium | GGCC_0272 | + | + | + | + | + | + | + | + | + | + | + | + | + |
| symbiosum | | | | | | | | | | | | | | |
| Bacteroides | GGCC_0301 | AA | + | + | + | + | + | + | + | + | + | + | + | + |
| uniformis | | | | | | | | | | | | | | |
| Bitterella | GGCC_0305 | AA | AA | + | + | + | + | + | + | + | + | AA | + | AA |
| massiliensis | | | | | | | | | | | | | | |
| Barnesiella sp. | GGCC_0306 | AA | + | + | + | + | + | + | + | + | + | + | + | + |

| | Val | Iso | Leu | His | Lys | Cys | Met | SAM | Fol | Gln | PAN | Nia | Thi | Rib | |
|---|---|---|---|---|---|---|---|---|---|---|---|---|---|---|---|
| Bacteroides | + | + | + | + | + | + | + | + | + | + | + | + | + | + | |
| xylanisolvens | | | | | | | | | | | | | | | |
| Clostridium | + | + | + | + | + | + | + | + | + | + | + | + | + | + | |
| butyricum | | | | | | | | | | | | | | | |
| Clostridium | + | + | + | + | + | + | + | + | + | + | AA | + | + | AA | |
| scindens | | | | | | | | | | | | | | | |
| Intestinimonas | + | + | + | + | + | + | + | + | AA | + | + | AA | AA | AA | |
| butyriciproducens | | | | | | | | | | | | | | | |
| Eubacterium | + | + | + | + | + | + | + | + | + | + | + | + | + | + | + |
| callanderi | + | + | + | + | + | + | + | + | AA | + | + | + | + | AA | + |
| Extibacter sp. | + | + | + | + | + | + | + | + | + | + | + | + | + | + | + |
| Akkermansia sp. | + | + | + | + | + | + | + | + | + | + | + | + | + | + | + |
| Clostridium | + | + | + | AA | + | + | + | + | + | + | AA | + | + | + | AA |
| symbiosum | | | | | | | | | | | | | | | |
| Bacteroides | + | + | + | + | + | + | + | + | + | + | + | + | + | + | AA |
| uniformis | | | | | | | | | | | | | | | |
| Bitterella | + | + | AA | AA | + | + | + | + | AA | + | AA | + | + | AA | AA |
| massiliensis | | | | | | | | | | | | | | | |
| Barnesiella sp. | + | + | + | + | + | + | + | + | + | + | + | + | + | + | AA |

The strain of Akkermansia species—like strain GGCC_0220 was deposited on 14 Oct. 2020 under the terms of the Budapest Treaty on the International Recognition of the Deposit of Microorganisms for the Purposes of Patent Procedure at the American Type Culture Collection (ATCC) in Manassas, Virginia, USA and bears the Patent Accession No. PTA-126870. This strain of Akkermansia was determined to be a new Akkermania-like species based on whole genome DNA average nucleotide identity (ANI). Specifically, this strain was determined to be distinct from strains of the species Akkermansia muciniphila based on having approximately 10% difference in whole genome DNA sequence compared to the Akkermansia muciniphila type strain ATCC BAA-835 as determined based on ANI. It is generally accepted in the field that ANI values around 95% correspond to the 70% DNA—DNA hybridization cut-off value, which is widely used to delineate archaeal and bacterial species (Arahal, 2014. Chapter 6—Whole-Genome Analyses: Average Nucleotide Identity. Methods in Microbiology 41: 103-122). In addition, whole genome sequence analysis of this strain reveals that it has additional properties that make it phenotypically and functionally distinct from the type strain Akkermansia muciniphila ATCC BAA-835. These properties include the synthesis of heme from cobalamine (vitamin B12) and the uptake of heme, the synthesis of a unique glycopolysaccharide with putative immunological properties, and the uptake and metabolism of 2-aminoethylphosphonate.

The strain of Bacteroides uniformis—like strain GGCC_0301 was deposited on 14 Oct. 2020 under the terms of the Budapest Treaty on the International Recognition of the Deposit of Microorganisms for the Purposes of Patent Procedure at the American Type Culture Collection (ATCC) in Manassas, Virginia, USA and bears the Patent Accession No. PTA-126872.

The strain of Bacteroides xylanisolvens—like strain GGCC_0124 was deposited on 14 Oct. 2020 under the terms of the Budapest Treaty on the International Recognition of the Deposit of Microorganisms for the Purposes of Patent Procedure at the American Type Culture Collection (ATCC) in Manassas, Virginia, USA and bears the Patent Accession No. PTA-126864.

The strain of Barnesiella species—like strain GGCC_0306 was deposited on 14 Oct. 2020 under the terms of the Budapest Treaty on the International Recognition of the Deposit of Microorganisms for the Purposes of Patent Procedure at the American Type Culture Collection (ATCC) in Manassas, Virginia, USA and bears the Patent Accession No. PTA-126874. This strain of Barnesiella was determined to be a new species, with its 16S rRNA gene having only 93% identity to the 16S rRNA genes of the closest related strains Barnesiella viscericola C46 and Barnesiella intestinihominis JCM 15079.

The strain of Bitterella massiliensis—like strain GGCC_0305 was deposited on 14 Oct. 2020 under the terms of the Budapest Treaty on the International Recognition of the Deposit of Microorganisms for the Purposes of Patent Procedure at the American Type Culture Collection (ATCC) in Manassas, Virginia, USA and bears the Patent Accession No. PTA-126873.

The strain of Clostridium butyricum—like strain GGCC_0151 was deposited on 14 Oct. 2020 under the terms of the Budapest Treaty on the International Recognition of the Deposit of Microorganisms for the Purposes of Patent Procedure at the American Type Culture Collection (ATCC) in Manassas, Virginia, USA and bears the Patent Accession No. PTA-126865.

The strain of Clostridium scindens—like strain GGCC_0168 was deposited on 14 Oct. 2020 under the terms of the Budapest Treaty on the International Recognition of the Deposit of Microorganisms for the Purposes of Patent Procedure at the American Type Culture Collection (ATCC) in Manassas, Virginia, USA and bears the Patent Accession No. PTA-126866.

The strain of Clostridium symbiosum—like strain GGCC_0272 was deposited on 14 Oct. 2020 under the terms of the Budapest Treaty on the International Recognition of the Deposit of Microorganisms for the Purposes of Patent Procedure at the American Type Culture Collection (ATCC) in Manassas, Virginia, USA and bears the Patent Accession No. PTA-126871.

The strain of Eubacterium callanderi—like strain GGCC_0197 was deposited on 14 Oct. 2020 under the terms of the Budapest Treaty on the International Recognition of the Deposit of Microorganisms for the Purposes of Patent Procedure at the American Type Culture Collection (ATCC) in Manassas, Virginia, USA and bears the Patent Accession No. PTA-126868.

The strain of Extibacter species—like strain GGCC_0201 was deposited on 14 Oct. 2020 under the terms of the Budapest Treaty on the International Recognition of the Deposit of Microorganisms for the Purposes of Patent Procedure at the American Type Culture Collection (ATCC) in Manassas, Virginia, USA and bears the Patent Accession No. PTA-126869. This strain of Extibacter was determined to be a new species, with its 16S rRNA gene having only 97% and 95% identity to the 16S rRNA genes of the closest related strains Extibacter muris strain 40cc-B-5824-ARE and Faecalicatena contorta strain DSM 3982, respectively.

The strain of Intestinimonas butyriciproducens—like strain GGCC_0179 was deposited on 14 Oct. 2020 under the terms of the Budapest Treaty on the International Recognition of the Deposit of Microorganisms for the Purposes of Patent Procedure at the American Type Culture Collection (ATCC) in Manassas, Virginia, USA and bears the Patent Accession No. PTA-126867.

The GUT-108 consortium significantly reduced inflammation in a clinically relevant mouse model of chronic immune-mediated colitis. EXAMPLE 2 herein describes evaluation of the efficacy of the GUT-108 consortium to treat an animal model of IBD characterized by chronic T-cell mediated inflammation. The preclinical study uses a validated animal model based on ex-germ-free (sterile) Il10^{-/-} 129SvEv mice where colitis is induced by inoculating the animals with human donor stool which results after 2 weeks in a moderate to severe chronic, bacterial antigen-specific Th1/Th17 driven inflammatory immune response. Mice were treated with GUT-108 consortium 2 weeks after administration of the human donor stool in a therapeutic protocol (i.e., delayed therapy after onset of disease). In addition, to evaluate the efficacy of the GUT-108 consortium to induce an anti-inflammatory IL-10 response, EXAMPLE 2 describes using a validated animal model based on eGFP-reporter (Il10⁺/^{eGFP}) C57BL/6J mice (also referred to as VertX reporter mice). In these mice, which have a wild-type IL-10 phenotype, induction of IL-10 synthesis can be visualized by expression of the eGFP reporter.

GUT-108 can be successfully established in gnotobiotic mice models. GUT-108 gavaged into two different germ-free mice models, Il-10⁺/^{eGFP} VertX reporter and Il10^{-/-} mice, established engraftment of all strains, as illustrated in FIGS. 1A and 1B.

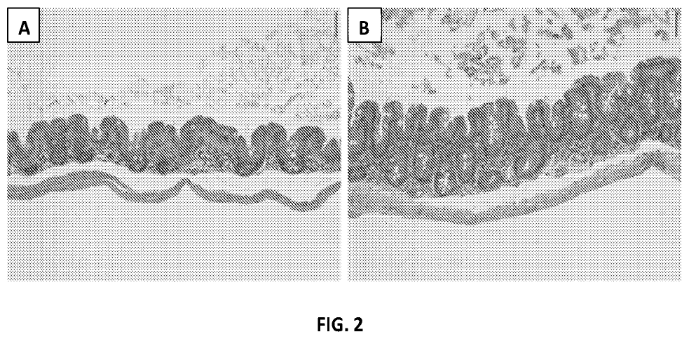

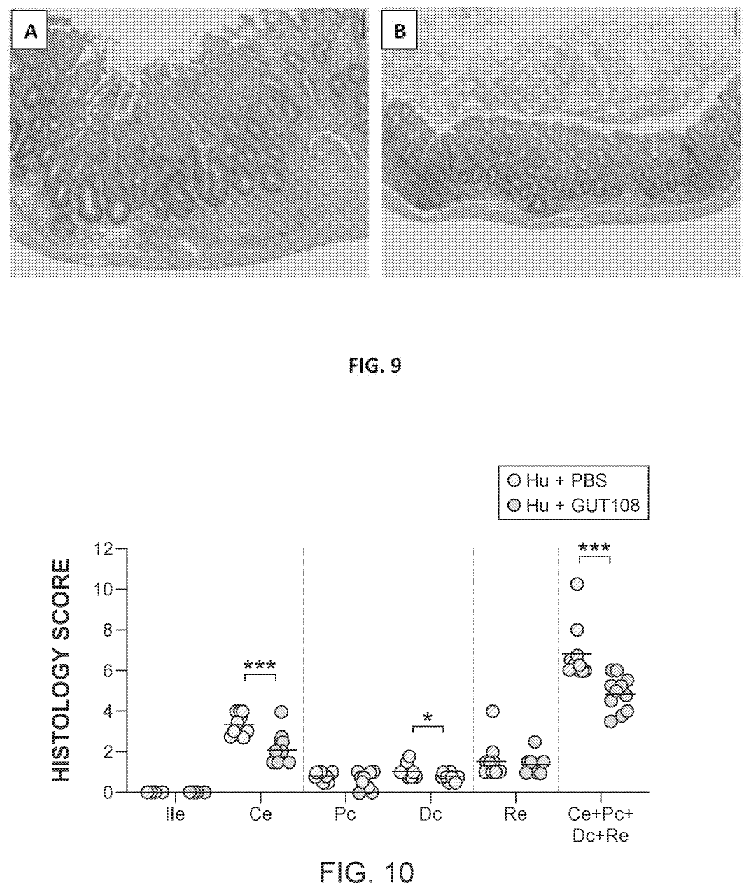

The therapeutic effect of GUT-108 is shown by visual evaluation of the degree of tissue inflammation in representative tissue samples taken from the cecum of the Il10^{-/-} knock-out mice. FIGS. 2A and 2B are representative histology images of cecum tissue from an ex-germ-free Il10^{-/-} mouse model of IBD treated with PBS as a control and with GUT 108, respectively, and show that treatment with GUT-108 did not induce colitis in the susceptible germ-free Il10^{-/-} mice. FIGS. 9A and 9B are representative distal colonic photomicrographs of H&E-stained tissue in an ex-germ-free Il10^{-/-} mouse model of IBD, where FIG. 9A is from a mouse that received PBS as a placebo two weeks after inoculation with human stool to induce colitis and FIG. 9B is from a mouse treated with GUT-108 two weeks after inoculation with human stool to induce colitis. The figures show the effectiveness of GUT-108 in reducing the induced inflammation in the cecum tissue relative to the PBS control.

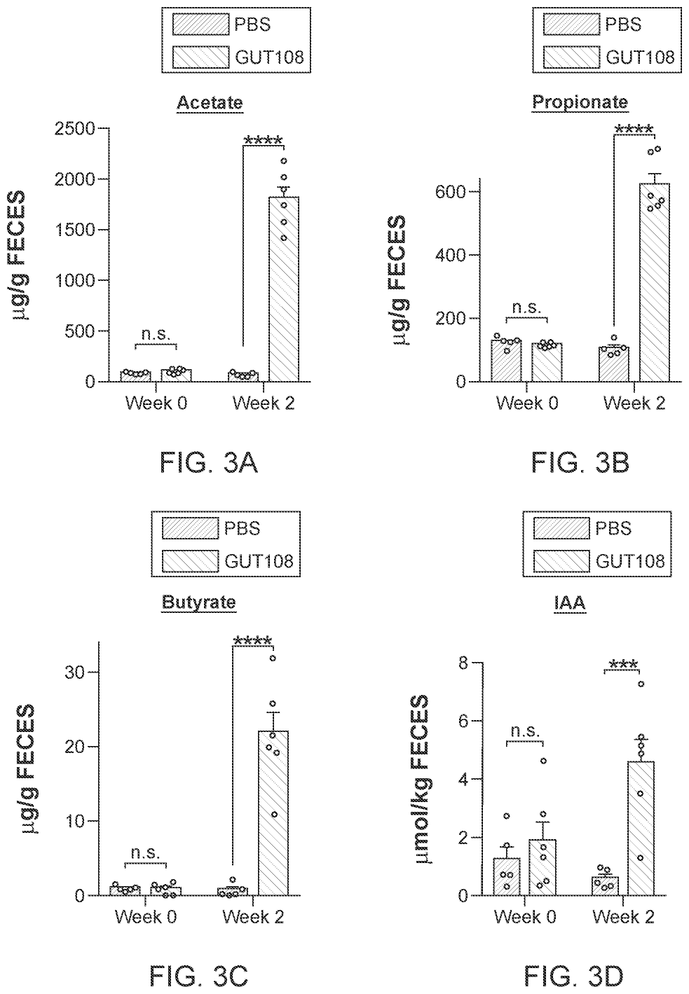

In addition to the observable reduced inflammation, GUT-108 colonization of gnotobiotic Il10^{-/-} knock-out mice promoted a healthy gut microbiome with key functionalities for short chain fatty acid (SCFA) synthesis, especially synthesis of acetate and propionate, and IAA synthesis being restored, as shown in FIGS. 3A-3D.

It is noted that four GUT-108 strains, Clostridium butyricum GGCC_0151, Intestinimonas butyriciproducens GGCC_0179, Bitterella massiliensis GGCC_0305 and Barnesiella sp. GGCC_0306 were established in the mouse gut at levels below 1% (see FIG. 1). The significance of the relatively low abundance of these four strains is unclear given the effectiveness of GUT-108 in reducing the induced inflammation in the mouse cecum tissue and promoting a healthy gut microbiome with key functionalities including SCFA synthesis. However, the key metabolic phenotypes of these four strains were likely also provided by other members of the GUT-108 consortium, due to the designed redundancies in metabolic interdependencies and therapeutic functions in GUT-108.

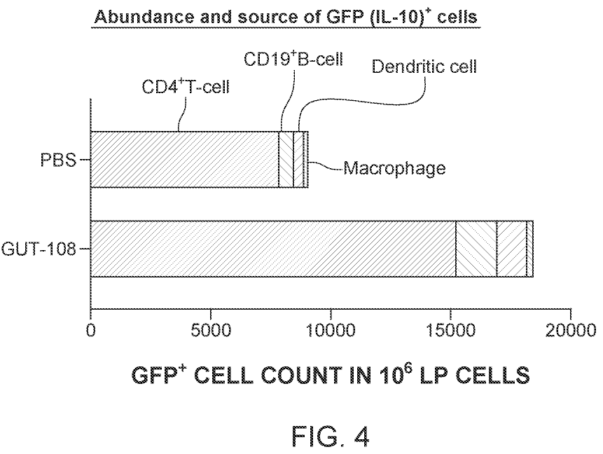

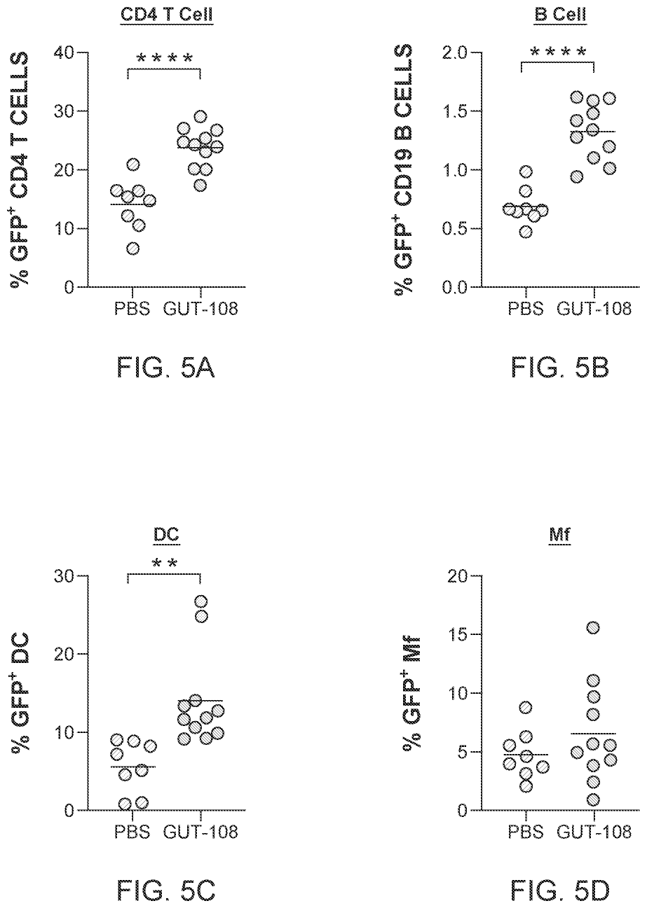

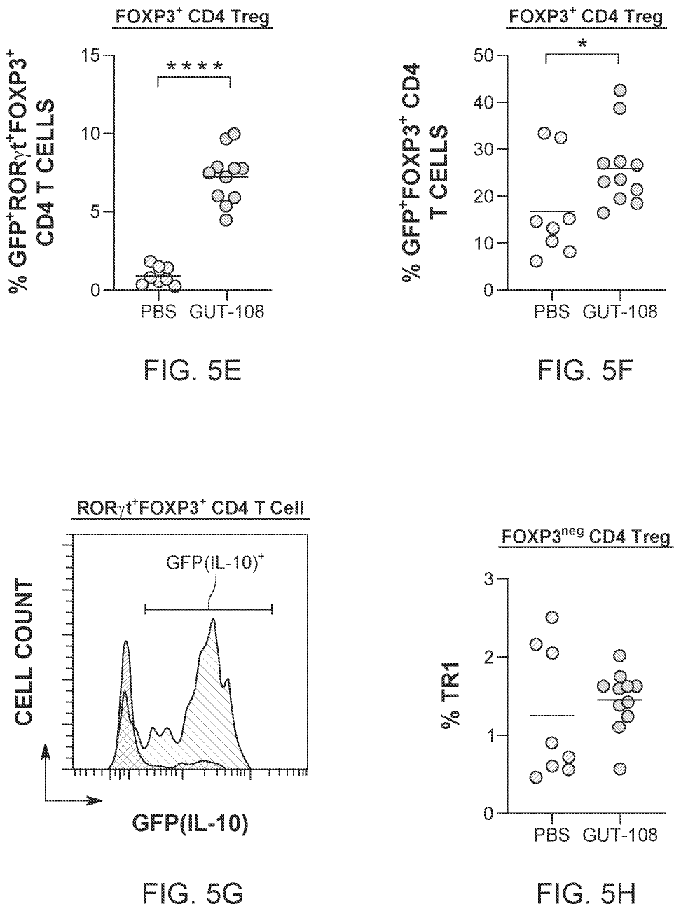

The results in FIGS. 4 and FIGS. 5A-5H show the ability of GUT-108 to induce the anti-inflammatory IL10 immune response by comparing the type and levels of IL-10 synthesizing immune cells in eGFP-reporter (Il10⁺/^{eGFP}) mice treated with either GUT-108 or PBS control. Mice treated with GUT-108 had increased levels of CD4+ GFP+(IL-10+) T cells and Treg cells (FIG. 4), especially FOXP3+RORγt+ GFP+(IL-10+) CD4+ Treg cells (FIGS. 5A-5H). The increase in IL-10+ producing CD4+ Treg cells is consistent with a low level of inflammation when germ free mice are exposed to microorganisms. In addition to different types of IL-10 synthesizing CD4+ Treg cells, elevated levels were observed of IL-10 synthesizing B cells, Dendritic Cells (DC), and Macrophages (FIGS. 4 and 5A-5H).

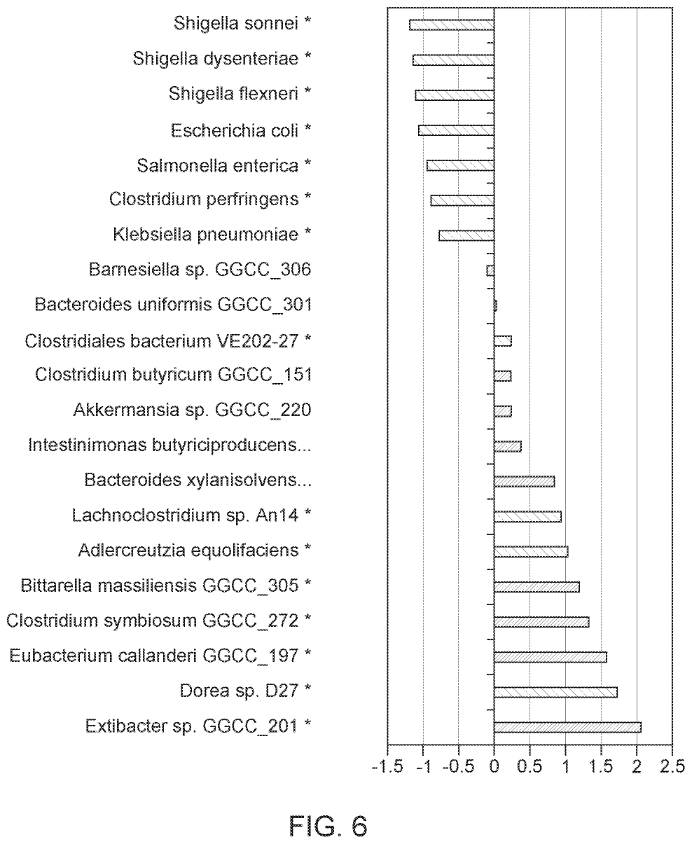

Metagenome analysis showed that therapeutic application of GUT-108 beginning 2 weeks after human fecal colonization to induce colitis resulted in engraftment of all GUT-108 strains except Clostridium scindens GGCC_0168 in the gut microbiome of the Il10^{-/-} mice. In addition and unexpectedly, therapeutic application of GUT-108 increased the abundance of beneficial resident Clostridium strains (Clusters IV and XIVa) that are not part of the GUT 108 consortium and decreased by 90% the abundance of pathogenic Enterobacteriaceae from 5% to below 0.5% (FIG. 6). An eight-fold decrease in the levels of the pathogenic Clostridium perfringens was also observed after treatment with GUT-108 (FIG. 6). These data indicate that administering GUT-108 can promote restoration of a healthy gut microbiome in a subject suffering from or at risk for an inflammatory condition. Thus, a method is provided that includes administering a composition of the present disclosure to a subject to restore a healthier gut microbiome, wherein a healthier gut microbiome can include one or both an increase in the abundance of beneficial Clostridium strains, especially Lachnospiraceae including Dorea species and Lachnoclostridium species that are not GUT-108 constituents, and a decrease in the abundance of pathogenic strains including enterohemorrhagic, enteroaggregative, enterotoxigenic and adherent invasive Escherichia coli, Klebsiella, Salmonella, and Shigella species and pathogenic Clostridium species such as, for example, Clostridium perfringens and Clostridium difficile.

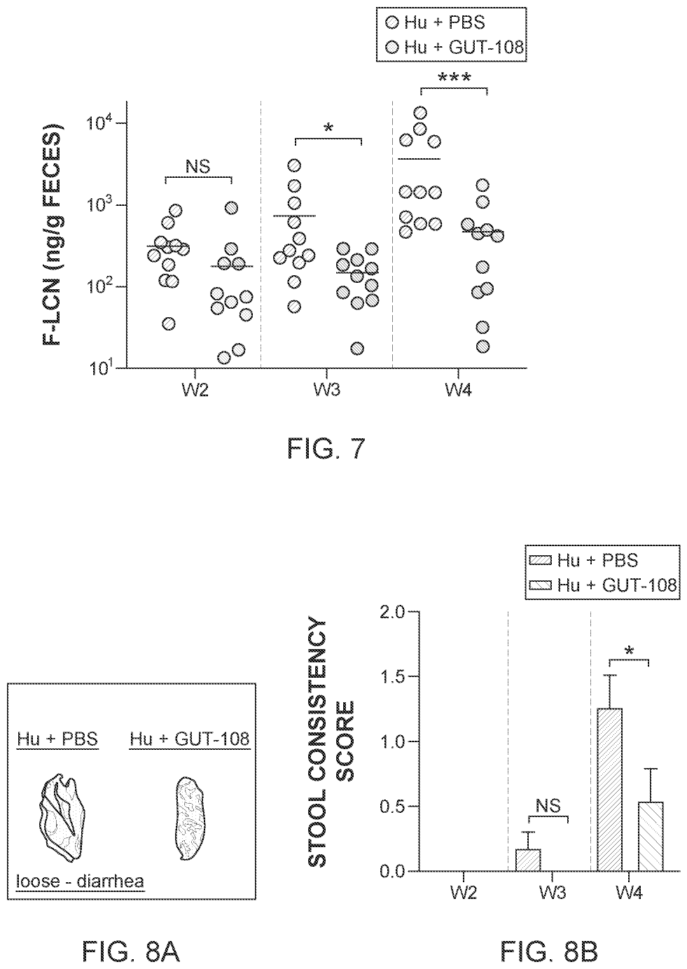

Progression of inflammation based on levels of lipocalin 2 secreted in the stool of the Il10^{-/-} knock-out mice as a function of time after gavage with human fecal material (Hu+PBS) and human fecal material plus the GUT-108 consortium in the therapeutic protocol (Hu+GUT-108) is illustrated in FIG. 7. The significant reduction after three and four weeks of the average lipocalin 2 levels in the stool of the Il10^{-/-} knock-out mice treated with GUT-108 indicates a decrease in colonic inflammation as compared to the control, showing the therapeutic effect of the GUT-108 consortium to treat chronic colitis. The therapeutic effect of GUT-108 is further confirmed by the reduction in stool consistency scores shown in FIGS. 8A and 8B for the Il10^{-/-} knock-out mice treated with GUT-108. Thus, a method is provided that includes administering a composition of the present disclosure to a subject to restore a healthier gut microbiome, wherein a healthier gut microbiome can result in a reduction in lipocalin 2 levels (or the human equivalent calprotectin where in clinical trials a decrease in calprotectin is considered an indication of a reduction in inflammation of the colonic tissue) and in a reduced stool consistency score.

The degree of colitis is shown by blinded histological scoring of sections of colon and cecum from the IL-10^{-/-} mice colonized with human fecal microbiota in FIG. 10. The results show a statistically significant decrease in blinded histology score for the mice treated with GUT-108, as compared to the mice that did not receive GUT-108. This further confirms that the application of GUT-108 two weeks after the initial gavage with human stool resulted in a reversal of established inflammation, showing the therapeutic effect of GUT-108 on colitis.

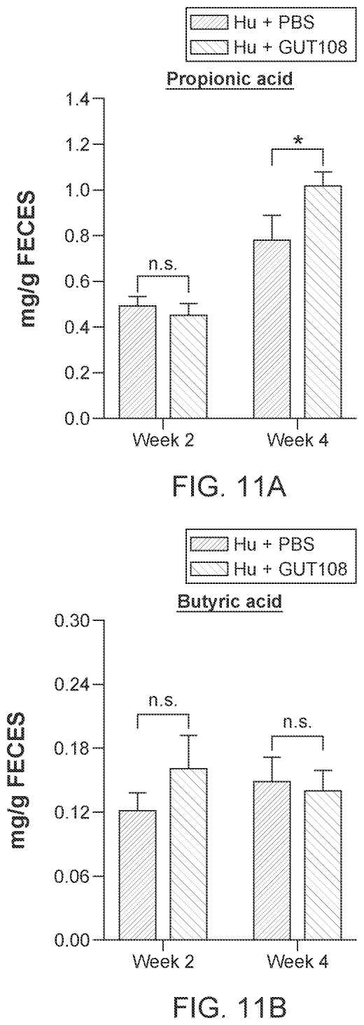

To confirm the restoration of normal gut microbiome functionality, levels of various metabolites were analyzed in the stool samples of the Il10^{-/-} knock-out mice colonized with human fecal microbiota. The results of FIGS. 11A-11B, 12A-12C, and 13A-13B show that compared to treatment with PBS, therapeutic treatment with GUT-108 (four applications over a two-week period after the onset of colitis induced by gavage with the human fecal material) resulted in the increase of levels of various secondary metabolites with beneficial anti-inflammatory properties. Specifically, levels of propionate, indole and its derivatives indole propionate (IPA) and indole acetate (IAA), and secondary bile acids deoxycholic acid (DCA) and lithocholic acid (LCA) were increased. Propionate is a primary nutrient source for colonic epithelial cells, as well as important for T cell induction. Indole propionate (IPA) is a potent AhR pathway agonist that is critical in controlling epithelial barrier integrity. Deconjugation and conversion of bile salts into secondary bile acids lithocholic acid and deoxycholic acid with therapeutic properties is essential to restore intestinal epithelial stem cell function, increase colonic RORγ+ Treg cell counts that ameliorate host susceptibility to colitis, and stimulate differentiation to Tregs and inhibit Th17 cells. Lithocholic acid has also been reported to have anti-aging properties due to its effect on mitochondrial lipid composition and energy processes, while deoxycholic acid is a strong antimicrobial with potent mode of action against microbial infections by pathogenic bacteria, including Clostridium perfringens and Clostridium difficile.

Increased levels of secondary metabolites in stool samples, more specifically the levels of propionate, indole and its derivatives indole propionate (IPA) and indole acetate (IAA), and secondary bile acids deoxycholic acid (DCA) and lithocholic acid (LCA), can be used as non-invasive biomarkers to monitor the progression of inflammation and to validate intervention strategies. In addition to their use to validate the establishment and performance of live biotherapeutic products, such as the GUT-108 consortium, these secondary metabolites have general applicability to monitor the long-term remission of patients treated with common therapeutics for conditions involving chronic inflammation, including corticosteroids and the therapeutics mentioned in Table 3. Thus, a method is provided that includes administering a composition of the present disclosure to a subject to restore a healthier gut microbiome, wherein a healthier gut microbiome can be characterized by an increase in the fecal levels of one or a combination of propionate, indole and its derivatives indole propionate (IPA) and indole acetate (IAA), and secondary bile acids deoxycholic acid (DCA) and lithocholic acid (LCA).

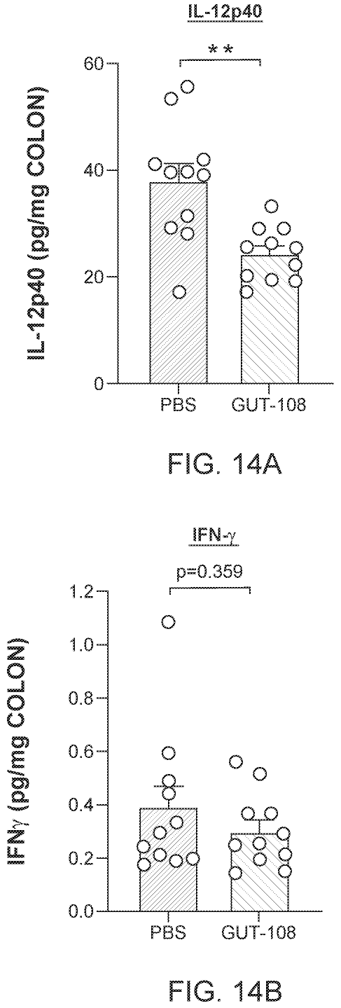

The spontaneous secretion levels of IL-12p40 and IFNγ are good indicators of the level of immune activation and inflammation of the colonic tissue. The results presented in FIGS. 14A and 14B show a decrease in IL-12p40 and IFNγ synthesis in colon tissue from gut cell cultures of the Il10^{-/-} knock-out mice colonized with human fecal microbiota and after treatment with GUT-108 further confirming the therapeutic effect of the GUT-108 consortium to treat chronic, immune-modulated colitis.

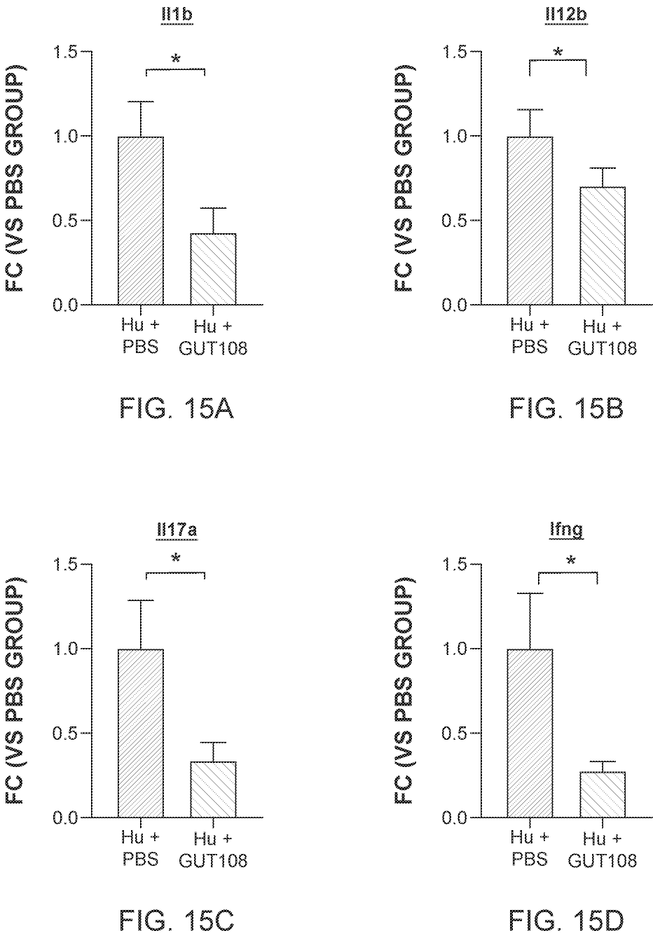

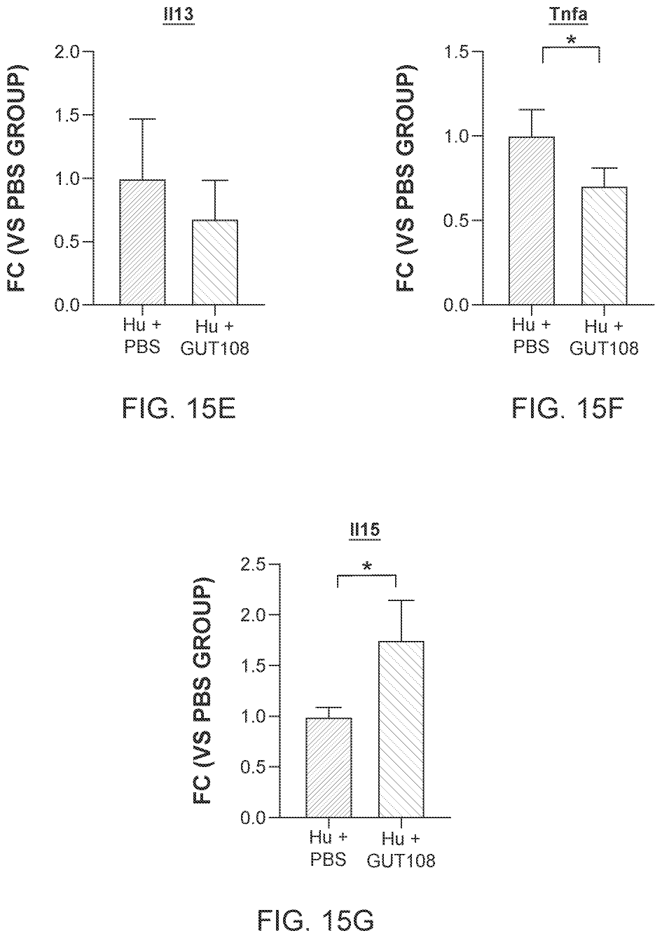

The results presented in FIGS. 15A-15F confirm the therapeutic effect of GUT-108 as shown by significant decrease in levels of expression of biosynthesis genes after treatment with GUT-108 for pro-inflammatory cytokines IL-1b, IL-12b, IL-13, IL-17α, IFNγ, and TNFα in the gut tissue of Il10^{-/-} knock-out mice colonized with human fecal microbiota; and after treatment with GUT-108 a significant increase in levels of expression of biosynthesis genes for homeostatic cytokine IL-15 in the gut tissue of Il10^{-/-} knock-out mice colonized with human fecal microbiota. Thus, a method is provided that includes administering a composition of the present disclosure to a subject to restore a healthier gut microbiome, wherein a healthier gut microbiome can be characterized by a decrease in the synthesis of proinflammatory cytokines.

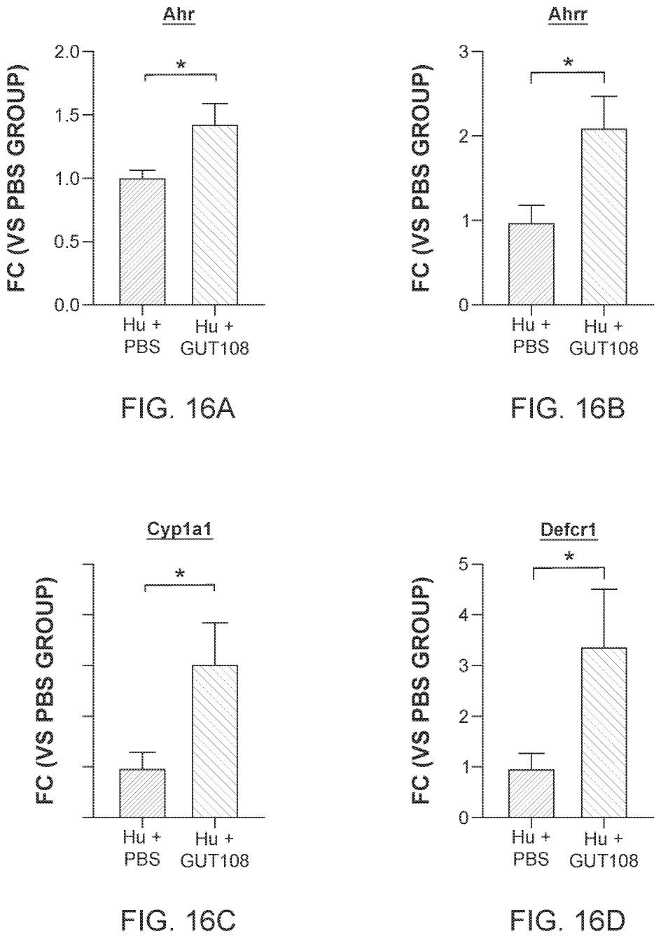

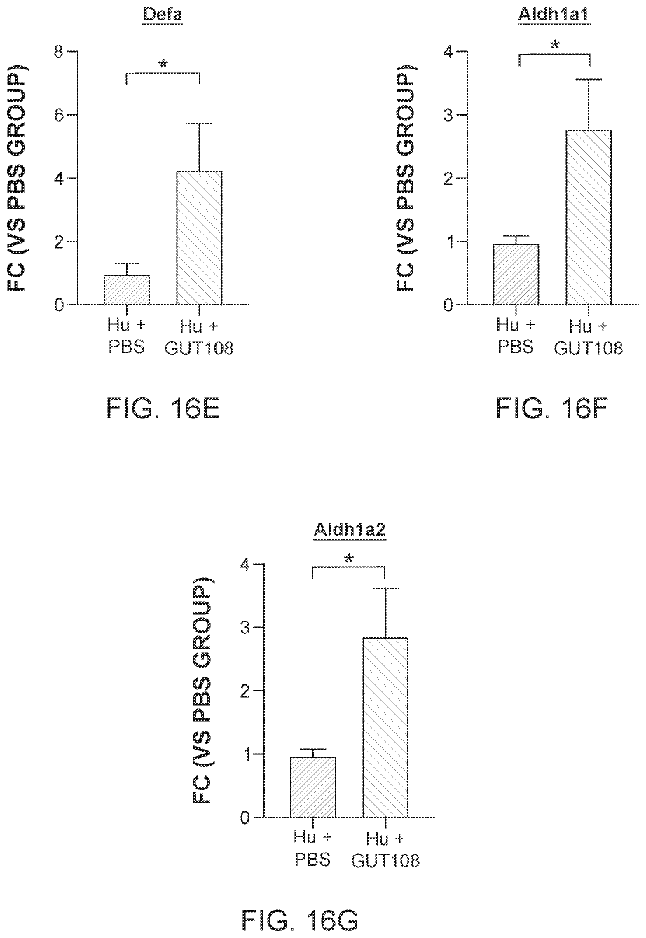

In addition to a decrease in the synthesis of proinflammatory cytokines, after treatment with GUT-108, increased colonic expression levels of genes for receptors and pathways implicated in mucosal healing was observed by Q-PCR in the gut tissue of Il10^{-/-} knock-out mice colonized with human fecal microbiota as shown in FIGS. 16A-16G. Unexpectedly, several protective pathway components that are decreased in IBD were upregulated in the Il10^{-/-} knock-out mouse gut tissue after treatment with GUT-108, including the Ahr pathway genes (the aryl hydrocarbon receptor (Ahr), the aryl hydrocarbon receptor repressor (AhrR) and the Cytochrome P450 Family 1 Subfamily A Member 1 (Cyp1A1) genes), the defensins DefCR1 and DefA, and the aldehyde dehydrogenases Aldh1A1 and Aldh1A2 that are part of the retinoic acid pathway, which is critical in regulating Wnt/β-catenin signaling. Thus, a method is provided that includes administering a composition of the present disclosure to a subject to restore a healthier gut microbiome, wherein a healthier gut microbiome can be characterized by an increase of beneficial functions associated with epithelial homeostasis and decreased IBD pathogenesis.

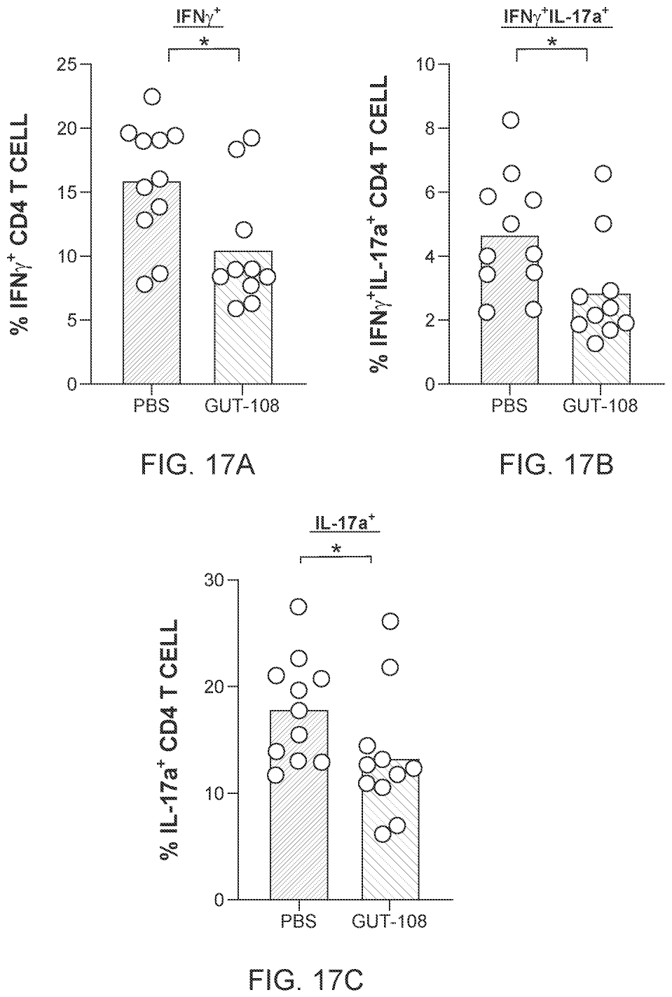

Evaluation of the anti-inflammatory effect of GUT-108 by determining the populations of CD4+ T cells after application of GUT-108 in the lamina propria of colon from the Il10^{-/-} knock-out mice colonized with human fecal microbiota is shown in FIGS. 17A-17C. Synthesis of pro-inflammatory cytokines IL-17α and IFNγ occurs in the lamina propria. The results in FIGS. 17A-17C show a significant decrease of IFN-γ+, IL-17α+ and IFN-γ+ IL-17α+ synthesizing CD4+ T cells after application of GUT-108 in the colonic lamina propria tissue of mice colonized with human fecal microbiota. These results further demonstrate the anti-inflammatory effect of GUT-108 to treat colitis, resulting in reversal of established inflammation. Thus, a method is provided that includes administering a composition of the present disclosure to a subject to restore a healthier gut microbiome, wherein a healthier gut microbiome can be characterized by a decrease in IFN-γ+, IL-17α+ and IFN-γ+ IL-17α+ synthesizing CD4+ T cells.

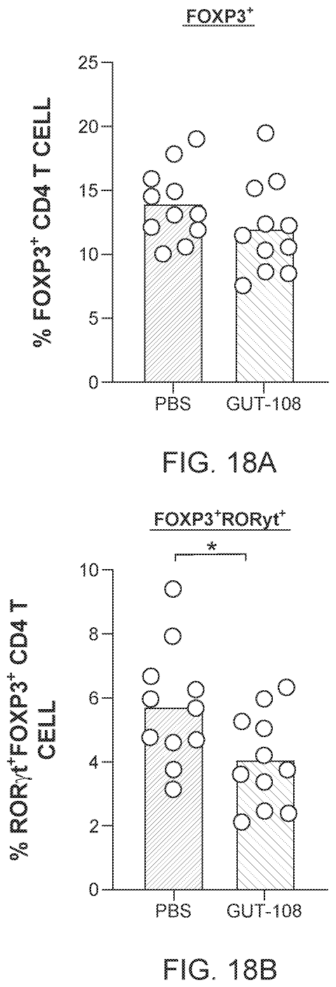

The data in FIGS. 18A-18B show an unexpected decrease after treatment with GUT-108 in FOXP3+ CD4+ T cells in colonic lamina propria cells from the Il10^{-/-} knockout mice colonized with human fecal microbiota. This result is unexpected because previously published studies, including the work on VE-202 by Atarashi et al (2013) in a similar Il10^{-/-} knockout mouse model of colitis, showed that introduction of a Live Biotherapeutic Product (LBP) resulted in an increase of FOXP3+ CD4+ T cells. This increase in FOXP3+ CD4+ T cells was defined as the key driver behind the inflammation control by these LBPs and part of homeostatic response to inflammation. It has been reported that there is an increased number of FOXP3+ CD4+ T cells in inflamed mucosal tissues in patients with active ulcerative colitis and Crohn's disease, and that levels of FOXP3+ CD4+ T cells go down to detection limits in non-inflamed tissues (Yu et al, 2007; Ban et al, 2008). Thus, the unexpected decrease in FOXP3+ CD4+ T cells observed after treatment with GUT-108 is indicative of superior control of inflammation by GUT-108 compared to other LBPs that have been tested in a similar mouse model. Thus, a method is provided that includes administering a composition of the present disclosure to a subject to restore a healthier gut microbiome, wherein a healthier gut microbiome can be characterized by a decrease in FOXP3⁺ CD4+ T cells indicative for reduced levels of epithelial inflammation.

The increased levels of various IL-10 synthesizing immune cell types points to a systemic anti-inflammatory response induced by GUT-108 that has broad applicability for treatment of a range of conditions characterized by a disease pathology including chronic inflammation of the gastrointestinal tract, such as Inflammatory Bowel Diseases (IBD), Irritable Bowel Syndrome (IBS), graft versus host disease, Parkinson's disease, ALS, systemic lupus erythematosus, asthma, multiple sclerosis, food allergies, fatty liver, hepatic encephalopathy, type-2 diabetes, metabolic syndrome and obesity, psoriasis, and well as aging related conditions caused by chronic inflammation, each of which have been shown to have a microbially-mediated inflammatory component. In addition to corticosteroid treatments, the current standard of care for autoimmune diseases including Ulcerative Colitis, Crohn's Disease, Ankylosing Spondylitis, Plaque Psoriasis, Psoriatic Arthritis, and Pouchitis, each of which have been shown to have an underlying microbially-mediated inflammatory component, are summarized in Table 3.

TABLE 3
Overview of commonly used drugs for the treatment of diseases of chronic inflammation. The disease condition,
the standard of care drugs for its treatment, and the mode of action of each drug are listed.

| Disease indication | Drug | Mode of action |
|---|---|---|
| Ulcerative colits | Humira (Abbvie) | mAb targets inflammatory pathway; anti TNFα |
| | Remicade (Janssen) | mAb targets inflammatory pathway; anti TNFα |
| | Entyvio (Takeda) | mAb blocking α4β7 integrin, which is recognized |
| | | by MAdCAM-1, thus blocking immune cell |
| | | recruitment in the gut epithelium |
| | Xeljanz (Pfizer) | Tofacitinib, sold under the tradename Xelianz, is a |
| | | JAK inhibitor that mainly interacts with JAK1 and |
| | | JAK3. JAK3 is the only JAK family member that |
| | | associates with just one cytokine receptor, |
| | | the common gamma chain, which is |
| | | exclusively used by the receptors for IL-2, |
| | | IL-4, IL-7, IL-9, IL-15 and IL-21. |
| | Methotrexate (MTX) - | A chemotherapy agent and immune system |
| | Trexall, | suppressant: |
| | Rheumatrex | https://www.ncbi.nlm.nih.gov/pmc/articles/PMC4133437/ |
| Crohn's | Humira (Abbvie) | anti TNFα |
| | Remicade (Janssen) | anti TNFα |
| | Entyvio (Takeda) | anti α4β7 integrin |
| | Methotrexate (MXT) | immune system suppressant |
| | Stelara (J&J) | mAb targets inflammatory pathway; anti |
| | | IL-12 and anti IL-23 |
| Ankylosing Spondylitis | Humira (Abbvie) | anti TNFα |
| | Remicade (Janssen) | anti TNFα |
| | naproxen (Naprosyn) | Nonsteroidal anti-inflammatory drug (NSAID) |
| | indomethacin (Indocin) | Nonsteroidal anti-inflammatory drug (NSAID) |
| Plaque Psoriasis | Humira (Abbvie) | anti TNFα |
| | Remicade (Janssen) | anti TNFα |
| | Taltz (Lilly) | mAb against IL-17 |
| | Otezla (Celgene) | selective inhibitor of the enzyme |
| | | phosphodiesterase 4 (PDE4) and inhibits |
| | | spontaneous production of TNFα |
| | Stelara (J&J) | anti IL-12 and anti IL-23 |
| Psoriatic Arthritis | Stelara (J&J) | anti IL-12 and anti IL-23 |
| | Humira (Abbvie) | anti TNFα |
| | Remicade (Janssen) | anti TNFα |
| | Methotrexate (MXT) | immune system suppressant |
| | Xeljanz (Pfizer) | JAK inhibitor that mainly interacts |
| | | with JAK1 and JAK3. |
| | Otezla (Celgene) | reduced TNFα |

| | |
|---|---|
| Pouchitis | Therapies used for pouchitis include antibiotics, budesonide |
| | enemas (a steroid drug), probiotics, biologic agents |
| | that target tumor necrosis factor (Humira, Remicade), |
| | glutamine suppositories, butyrate suppositories, bismuth |
| | enemas (diarrhea medication), allopurinol (a purine |
| | analogue drug), and tinidazole (an anti-parasitic drug). |
| | Overall objective is inflammation and infection control. |

In contrast to GUT-108, which targets the underlying causes of these diseases, especially intestinal infection and (gut epithelial) inflammation, the current standard of care drugs are targeting the downstream effects on immune pathways neutralizing a single cytokine or molecule, such as the elevated levels of the pro-inflammatory cytokines TNFα, IL-12 and IL-23, and IL-17. Furthermore, successful long-term remission by these drugs requires that dysbiosis of the gut microbiome, which is characteristic for these diseases, is corrected.

Based on the observed activity spectrum for GUT-108 and a comparison with current standard of care drugs that target the inflammatory response, GUT-108 can be used both preventatively and therapeutically for the treatment of ulcerative colitis, Crohn's disease, Ankylosing Spondylitis, Plaque Psoriasis, Psoriatic Arthritis and Pouchitis as a stand-alone therapeutic, lowering the expression of multiple pro-inflammatory cytokines; or as a companion therapy to corticosteroids and the drugs listed in Table 3 to reduce the treatment time to achieve remission, and to ensure long-term remission by correcting the underlying causes of the disease. Thus, a method is provided that includes administering a composition of the present disclosure to a subject to decrease inflammation, either as a stand-alone treatment or as an adjuvant therapy. The inflammation can be a result of one or more disorders including Inflammatory Bowel Diseases (IBD) like ulcerative colitis and Crohn's disease, Pouchitis, Irritable Bowel Syndrome (IBS), graft versus host disease, Parkinson's disease, ALS, multiple sclerosis, systemic lupus erythematosus, Ankylosing Spondylitis, asthma, food allergies, fatty liver, hepatic encephalopathy, Primary Sclerosing Cholangitis as a comorbidity for IBD, type-2 diabetes, metabolic syndrome and obesity, Plaque Psoriasis, Psoriatic Arthritis, and aging related conditions of chronic inflammation.

As shown in EXAMPLE 2, gavage with the GUT-108 consortium in a therapeutic protocol significantly lowered the level of inflammation in the Il10^{-/-} knockout mice selectively colonized with human donor stool. Ahr is identified as one of the functions whose gene expression levels can be significantly upregulated by the therapeutic application of GUT-108. Upregulation of Ahr has been associated with a positive outcome of various aging related conditions via its effect on various targets, including P-glycoprotein expression, fibroblast growth factors (Fgf15, Fgf21), various tight junction proteins (including occluding and claudin-5) and the differentiation and function of immune cells including Tcells, macrophages and dendritic cells. Ahr activation via the gut microbiome may activate these targets, which has been described to have a positive effect on the development of Alzheimer's and Non-Alzheimer's disease related dementia, Parkinson's disease, multiple sclerosis, Type-2 diabetes, obesity, hepatic steatosis, and aging related cardiovascular and neurologic conditions, all of which have been demonstrated as having an underlying gut microbiome component. Thus, in one embodiment, GUT-108 is provided as a therapeutic agent for the treatment of these condition. A method is provided that includes administering a composition of the present disclosure to a subject to treat one or more of Alzheimer's and Non-Alzheimer's disease related dementia, Parkinson's disease, multiple sclerosis, Type-2 diabetes, obesity, hepatic steatosis, and aging related cardiovascular and neurologic conditions.

The aldehyde dehydrogenases Aldh1A1 and Aldh1A2 were identified as one of the functions whose gene expression levels can be significantly upregulated by the therapeutic application of GUT-108. Upregulation of Aldh1A1 and Aldh1A2 has been associated with increased activity of the retinoic acid pathway, which is critical in regulating Wnt/β-catenin signaling. Studies indicate that reduced activity of the retinoic acid pathway relate to tumor development and cellular migration. Several cellular pathways, including Wnt/β-catenin signaling pathway, are related to cancer metastasis, and many reports have suggested that exaggerated Wnt signaling can lead to cancer initiation and progression in a wide range of human tissues, including colon cancer, which has higher occurrence in IBD patients compared to healthy individuals. Therefore, in one embodiment, a method is provided that includes administering a composition of the present disclosure to a subject having IBD to lower the risk of the subject developing colorectal cancer.

Without being bound to any particular mechanism of action, data indicate that GUT-108 may exert beneficial effects through a combination of multiple modes of action to treat the upstream causes of inflammation by correcting dysbiosis, activating various IL10 synthesizing immune cells, lowering inflammatory responses, and restoring normal bacterial metabolic profiles. These overlapping protective mechanisms may result in maintenance of long-term remission of IBD and other diseases characterized by a pro-inflammatory gut microbiome in a physiologic and safe manner. This is in contrast to most biologicals, which block downstream immune effector responses by neutralizing a single cytokine or molecule and induce immunosuppression that can be associated with increased infection and neoplasms.

The integrated protective mechanisms can make GUT-108 a promising therapy to treat a range of conditions having pathogenesis characterized by dysbiosis-mediated chronic intestinal inflammation and increased mucosal permeability. In addition to IBD, these conditions include Irritable Bowel Syndrome (IBS), graft versus host disease, Parkinson's disease, ALS, multiple sclerosis, systemic lupus erythematosus, asthma, food allergies, fatty liver, hepatic encephalopathy, type-2 diabetes, metabolic syndrome and obesity, psoriasis, as well as aging related conditions caused by chronic inflammation, each of which have been shown to have a microbially-mediated inflammatory component.

In other cases, conditions having pathogenesis characterized by dysbiosis-mediated chronic intestinal inflammation and increased mucosal permeability can be caused by infection with or overpopulation of pathogenic members of the Enterobacteriaceae and/or one or more pathogenic species of Clostridium including Clostridium difficile and Clostridium perfringens. Thus, treatment with GUT-108 can also be a promising therapy to treat infection and/or inflammation in a subject due to these causes.

In one embodiment, a composition is provided for treating inflammation in a subject or for improving the health of the microbiome of the subject, the composition comprising a biologically pure culture of each of: Akkermansia species—like strain GGCC_0220, Bacteroides uniformis—like strain GGCC_0301, Bacteroides xylanisolvens—like strain GGCC_0124, Barnesiella species—like strain GGCC_0306, Bitterella massiliensis—like strain GGCC_0305, Clostridium butyricum—like strain GGCC_0151, Clostridium scindens—like strain GGCC_0168, Clostridium symbiosum—like strain GGCC_0272, Eubacterium callanderi—like strain GGCC_0197, Extibacter species—like strain GGCC_0201 or Intestinimonas butyriciproducens—like strain GGCC_0179.

In one embodiment, a composition is provided for treating inflammation in a subject, or for improving the health of the microbiome of the subject, the composition comprising a biologically pure culture of two or more of: Akkermansia species—like strain GGCC_0220, Bacteroides uniformis—like strain GGCC_0301, Bacteroides xylanisolvens—like strain GGCC_0124, Barnesiella species—like strain GGCC_0306, Bitterella massiliensis—like strain GGCC_0305, Clostridium butyricum—like strain GGCC_0151, Clostridium scindens—like strain GGCC_0168, Clostridium symbiosum—like strain GGCC_0272, Eubacterium callanderi—like strain GGCC_0197, Extibacter species—like strain GGCC_0201 or Intestinimonas butyriciproducens—like strain GGCC_0179.

In one embodiment, a composition is provided for treating inflammation in a subject or for improving the health of the microbiome of the subject, the composition comprising a biologically pure culture of two or more of a bacterium having 97% sequence identity to the 16S rRNA gene sequence of: Akkermansia species—like strain GGCC_0220 (SEQ ID NO: 1), Bacteroides uniformis—like strain GGCC_0301 (SEQ ID NO: 2), Bacteroides xylanisolvens—like strain GGCC_0124 (SEQ ID NO: 3), Barnesiella species—like strain GGCC_0306 (SEQ ID NO: 4), Bitterella massiliensis—like strain GGCC_0305 (SEQ ID NO: 5), Clostridium butyricum—like strain GGCC_0151 (SEQ ID NO: 6), Clostridium scindens—like strain GGCC_0168 (SEQ ID NO: 7), Clostridium symbiosum—like strain GGCC_0272 (SEQ ID NO: 8), Eubacterium callanderi—like strain GGCC_0197 (SEQ ID NO: 9), Extibacter species—like strain GGCC_0201 (SEQ ID NO: 10) or Intestinimonas butyriciproducens—like strain GGCC_0179 (SEQ ID NO: 11).

In one embodiment, a composition is provided for treating inflammation in a subject or for improving the health of the microbiome of the subject, the composition comprising a biologically pure culture of two or more of: a) a bacterium having 97% sequence identity to the 16S rRNA gene sequence of Akkermansia species—like strain GGCC_0220 (SEQ ID NO: 1) and genetic material encoding for synthesis of propionate, synthesis of 4-amino-butyrate (gamma-aminobutyric acid; GABA), and synthesis of indole or indole-containing secondary metabolites; b) a bacterium having 97% sequence identity to the 16S rRNA gene sequence of Bacteroides uniformis—like strain GGCC_0301 (SEQ ID NO: 2) and genetic material encoding for synthesis of propionate, synthesis of GABA, and synthesis of indole or indole-containing secondary metabolites; c) a bacterium having 97% sequence identity to the 16S rRNA gene sequence of Bacteroides xylanisolvens—like strain GGCC_0124 (SEQ ID NO: 3) and genetic material encoding for synthesis of propionate, synthesis of GABA, and synthesis of indole or indole-containing secondary metabolites; d) a bacterium having 99% sequence identity to the 16S rRNA gene sequence of Barnesiella species—like strain GGCC_0306 (SEQ ID NO: 4) and genetic material encoding for synthesis of propionate and synthesis of GABA; e) a bacterium having 97% sequence identity to the 16S rRNA gene sequence of Bitterella massiliensis—like strain GGCC_0305 (SEQ ID NO: 5), and genetic material encoding for 3α-hydroxy steroid dehydrogenase and 3β-hydroxy steroid dehydrogenase; f) a bacterium 97% sequence identity to the 16S rRNA gene sequence of Clostridium butyricum—like strain GGCC_0151 (SEQ ID NO: 6) and genetic material encoding for synthesis of butyrate; g) a bacterium having 97% sequence identity to the 16S rRNA gene sequence of Clostridium scindens—like strain GGCC_0168 (SEQ ID NO: 7) and genetic material encoding for bile acid 7-alpha-dehydratase activity; h) a bacterium having 97% sequence identity to the 16S rRNA gene sequence of Clostridium symbiosum—like strain GGCC_0272 (SEQ ID NO: 8) and genetic material encoding for synthesis of butyrate; i) a bacterium having 97% sequence identity to the 16S rRNA gene sequence of Eubacterium callanderi-like strain GGCC_0197 (SEQ ID NO: 9) and genetic material encoding for synthesis of butyrate, synthesis of GABA, and bile acid 7-alpha-dehydratase activity; j) a bacterium having 97% sequence identity to the 16S rRNA gene sequence of Extibacter species—like strain GGCC_0201 (SEQ ID NO: 10) and genetic material encoding for bile acid 7-alpha-dehydratase activity; and k) a bacterium having 97% sequence identity to the 16S rRNA gene sequence of Intestinimonas butyriciproducens—like strain GGCC_0179 (SEQ ID NO: 11) and genetic material encoding for synthesis of butyrate.

In one embodiment of the present disclosure, a composition for use in a method of benefiting the health of animal or a human is provided comprising a biologically pure culture of each of: Akkermansia species—like strain GGCC_0220, Bacteroides uniformis—like strain GGCC_0301, Bacteroides xylanisolvens—like strain GGCC_0124, Clostridium butyricum—like strain GGCC_0151, Clostridium scindens—like strain GGCC_0168, Clostridium symbiosum—like strain GGCC_0272, Eubacterium callanderi—like strain GGCC_0197, Extibacter species—like strain GGCC_0201 or Intestinimonas butyriciproducens—like strain GGCC_0179.

In one embodiment of the present disclosure, a composition for use in a method of benefiting the health of animal or a human is provided comprising a biologically pure culture of each of a bacterium having 97% sequence identity to the 16S rRNA gene sequence of: Akkermansia species—like strain GGCC_0220 (SEQ ID NO: 1), Bacteroides uniformis—like strain GGCC_0301 (SEQ ID NO: 2), Bacteroides xylanisolvens—like strain GGCC_0124 (SEQ ID NO: 3), Clostridium butyricum—like strain GGCC_0151 (SEQ ID NO: 6), Clostridium scindens—like strain GGCC_0168 (SEQ ID NO: 7), Clostridium symbiosum—like strain GGCC_0272 (SEQ ID NO: 8), Eubacterium callanderi—like strain GGCC_0197 (SEQ ID NO: 9), Extibacter species—like strain GGCC_0201 (SEQ ID NO: 10) or Intestinimonas butyriciproducens—like strain GGCC_0179 (SEQ ID NO: 11).

In one embodiment of the present disclosure, a composition for use in a method of benefiting the health of animal or a human is provided comprising each of:
a) a biologically pure culture of a bacterium having 97% sequence identity to the 16S rRNA gene sequence of Akkermansia species—like strain GGCC_0220 (SEQ ID NO: 1), said bacterium comprising genetic material encoding for synthesis of propionate, synthesis of 4-amino-butyrate (gamma-aminobutyric acid; GABA), and synthesis of indole or indole-containing secondary metabolites;
b) a biologically pure culture of a bacterium having 97% sequence identity to the 16S rRNA gene sequence of Bacteroides uniformis—like strain GGCC_0301 (SEQ ID NO: 2), said bacterium comprising genetic material encoding for synthesis of propionate, synthesis of GABA, and synthesis of indole or indole-containing secondary metabolites;
c) a biologically pure culture of a bacterium having 97% sequence identity to the 16S rRNA gene sequence of Bacteroides xylanisolvens—like strain GGCC_0124 (SEQ ID NO: 3), said bacterium comprising genetic material encoding for synthesis of propionate, synthesis of GABA, and synthesis of indole or indole-containing secondary metabolites;
d) a biologically pure culture of a bacterium 97% sequence identity to the 16S rRNA gene sequence of Clostridium butyricum—like strain GGCC_0151 (SEQ ID NO: 6), said bacterium comprising genetic material encoding for synthesis of butyrate;
e) a biologically pure culture of a bacterium having 97% sequence identity to the 16S rRNA gene sequence of Clostridium scindens—like strain GGCC_0168 (SEQ ID NO: 7), said bacterium comprising genetic material encoding for bile acid 7-alpha-dehydratase activity;
f) a biologically pure culture of a bacterium having 97% sequence identity to the 16S rRNA gene sequence of Clostridium symbiosum—like strain GGCC_0272 (SEQ ID NO: 8), said bacterium comprising genetic material encoding for synthesis of butyrate;
g) a biologically pure culture of a bacterium having 97% sequence identity to the 16S rRNA gene sequence of Eubacterium callanderi—like strain GGCC_0197 (SEQ ID NO: 9), said bacterium comprising genetic material encoding for synthesis of butyrate, synthesis of GABA, and bile acid 7-alpha-dehydratase activity;
h) a biologically pure culture of a bacterium having 97% sequence identity to the 16S rRNA gene sequence of Extibacter species—like strain GGCC_0201 (SEQ ID NO: 10), said bacterium comprising genetic material encoding for bile acid 7-alpha-dehydratase activity; and
i) a biologically pure culture of a bacterium having 97% sequence identity to the 16S rRNA gene sequence of Intestinimonas butyriciproducens—like strain GGCC_0179 (SEQ ID NO: 11) said bacterium comprising genetic material encoding for synthesis of butyrate.

The compositions of the presently disclosed subject matter can be formulated as pharmaceutical compositions. In one example, the pharmaceutical compositions may comprise one or more pharmaceutically acceptable carriers.

The compositions of the presently disclosed subject matter can include three, four, five, six, seven, eight, nine, ten, or eleven of the biologically pure cultures of the bacterial strains of GUT-108.

In the compositions of the presently disclosed subject matter, the biologically pure cultures of bacterial strains can have complementary auxotrophies to provide metabolic interdependency between all of the strain members in the consortium.

In the compositions of the presently disclosed subject matter, the biologically pure cultures of microbial strains can be in the form of spores or vegetative cells or a combination thereof. The biologically pure cultures of microbial strains be in the form of freeze-dried spores or vegetative cells or a combination thereof.

In one embodiment, the presently disclosed bacterial strains are formulated in freeze-dried form (otherwise referred to as “lyophilized”), For example, the presently disclosed compositions may comprise granules or gelatin capsules, for example hard gelatin capsules, comprising the bacterial strains of the invention.

Alternatively, the presently disclosed compositions may comprise live, active bacterial cultures.

The compositions of the presently disclosed subject matter can be formulated for oral or rectal administration. The compositions can be formulated for delivery to the intestine. The compositions of the presently disclosed subject matter can be in the form of a dry powder, tablet, or capsule.

For formulating the compositions, and particularly for formulating the compositions for oral administration, it is prefer able to use a composition which enables an efficient delivery of the composition to the intestine.

In one embodiment, the compositions are encapsulated to enable delivery of the bacterial strains to the intestine. Encapsulation protects the composition from degradation until delivery at the target location through, for example, rupturing with chemical or physical stimuli such as pressure, enzymatic activity, or physical disintegration, which may be triggered by changes in pH. Any appropriate encapsulation method may be used. Exemplary encapsulation techniques include entrapment within a porous matrix, attachment or adsorption on solid carrier surfaces, self-aggregation by flocculation or with cross-linking agents, and mechanical containment behind a microporous membrane or a microcapsule.

Compositions and methods which enable delivery to the intestine are not particularly limited and known compositions or methods can be employed as appropriate. Examples thereof include pH sensitive compositions, more specifically, enteric polymers which release their contents when the pH becomes alkaline after the enteric polymers pass through the stomach. When a pH sensitive composition is used for formulating the composition, the pH sensitive composition is preferably a polymer whose pH threshold of the decomposition of the composition is 6.8 to 7.5. Such a numeric value range is a range where the pH shifts toward the alkaline side at a distal portion of the stomach, and hence is a suitable range for use in the delivery to the intestine.

Another example of the composition enabling the delivery to the intestine is a composition which ensures the delivery to the intestine by delaying the release of the contents by approximately 3 to 5 hours, which corresponds to the small intestinal transit time. In an example of formulating a composition for delaying the release, a hydrogel is used as a shell. The hydrogel is hydrated and swells upon contact with gastrointestinal fluid, so that the contents are effectively released. In addition, the delayed release dosage units include compositions formulated with a material which coats or selectively coats. Examples of such a selective coating material include in vivo degradable polymers, gradually hydrolyzable polymers, gradually water-soluble polymers, and/or enzyme degradable polymers. A preferred coating material for efficiently delaying the release is not particularly limited, and examples thereof include cellulose-based polymers such as hydroxypropyl cellulose, acrylic add polymers and copolymers such as methacrylic add polymers and copolymers, and vinyl polymers and copolymers such as polyvinylpyrrolidone.

Examples of the composition enabling the delivery to the intestine further include bioadhesive compositions which specifically adhere to the intestinal mucosal membrane, and compositions into which a protease inhibitor is incorporated for protecting particularly a composition in the gastrointestinal tracts from decomposition due to an activity of a protease.

An example of a composition formulated for delivery to the intestine is a composition designed for delivery by pressure change in such a way that the contents of the composition are released by utilizing pressure changes caused by goner anon of gas from bacterial fermentation at a distal portion of the stomach. Such a formulation for delivery by pressure change is not particularly limited, and a more specific example thereof is a capsule which has the contents dispersed in a suppository base and which is coated with a hydrophobic polymer (for example, ethyl cellulose).

Another example of enabling the delivery to the intestine is formulation of the composition for specific decomposition by an enzyme (for example, a carbohydrate hydrolase or a carbohydrate reductase) present in the intestine. Such a formulation is not particularly limited, and more specific examples thereof include compositions formulated to include food components such as non-starch polysaccharides, amylose, xanthan gum, and azopolymers.

The composition may be formulated as a probiotic.

In one embodiment, the presently disclosed compositions include a therapeutically effective amount of a bacterial strain. A therapeutically effective amount of a bacterial strain is sufficient to exert a beneficial effect upon a patient. A therapeutically effective amount of a bacterial strain may be sufficient to result in delivery to and/or partial or total colonization of the patient's intestine.

In one embodiment, the compositions of the present disclosure are administered to the subject on a daily basis. A suitable daily dose of the bacteria, for example for an adult human, may be from about 1×10³ to about 1×10¹² colony forming units (CFU); for example, from about 1×10⁷ to about 1×10¹⁰ CFU; in another example from about 1×10⁶ to about 1×10¹⁰ CFU.

In certain embodiments, the composition contains the bacterial strain in an amount of from about 1×10⁶ to about 1×10¹¹ CFU/g, with respect to the weight of the composition; for example, from about 1×10³ to about 1×10¹⁰ CFU/g. The dose may be, for example, 1 g, 3 g, 5 g, and 10 g.

Typically, a probiotic, such as the compositions of the presently disclosed subject matter, is optionally combined with at least one suitable prebiotic compound. A prebiotic compound is usually a non-digestible carbohydrate such as an oligo- or polysaccharide, or a sugar alcohol, which is not degraded or absorbed in the upper digestive tract, Known probiotics include commercial products such as inulin and transgalacto-oligosaccharides.

In one embodiment, a method is provided for treating an inflammatory disorder in a subject, comprising: administering to the subject a composition of the present disclosure, wherein the administering results in a decrease in inflammation.

In one embodiment, a method is provided for improving the health of the microbiome of a subject, comprising: administering to the subject a composition of the present disclosure, wherein the administering results in an improvement in the health of the subject's microbiome.

In one embodiment, a method is provided for treating an inflammatory disorder in a subject, comprising: administering to the subject a composition comprising a biologically pure culture of two or more of: Akkermansia species—like strain GGCC_0220, Bacteroides uniformis—like strain GGCC_0301, Bacteroides xylanisolvens—like strain GGCC_0124, Barnesiella species—like strain GGCC_0306, Bitterella massiliensis—like strain GGCC_0305, Clostridium butyricum—like strain GGCC_0151, Clostridium scindens—like strain GGCC_0168, Clostridium symbiosum—like strain GGCC_0272, Eubacterium callanderi—like strain GGCC_0197, Extibacter species—like strain GGCC_0201 or Intestinimonas butyriciproducens—like strain GGCC_0179.

In one embodiment, a method is provided for treating an inflammatory disorder in a subject, comprising: administering to the subject a composition a biologically pure culture of two or more of a bacterium having 97% sequence identity to the 16S rRNA gene sequence of: Akkermansia species—like strain GGCC_0220 (SEQ ID NO: 1), Bacteroides uniformis—like strain GGCC_0301 (SEQ ID NO: 2), Bacteroides xylanisolvens—like strain GGCC_0124 (SEQ ID NO: 3), Barnesiella species—like strain GGCC_0306 (SEQ ID NO: 4), Bitterella massiliensis—like strain GGCC_0305 (SEQ ID NO: 5), Clostridium butyricum—like strain GGCC_0151 (SEQ ID NO: 6), Clostridium scindens—like strain GGCC_0168 (SEQ ID NO: 7), Clostridium symbiosum—like strain GGCC_0272 (SEQ ID NO: 8), Eubacterium callanderi—like strain GGCC_0197 (SEQ ID NO: 9), Extibacter species—like strain GGCC_0201 (SEQ ID NO: 10) or Intestinimonas butyriciproducens—like strain GGCC_0179 (SEQ ID NO: 11).

In one embodiment, a method is provided for treating an inflammatory disorder in a subject, comprising administering to the subject a composition comprising a biologically pure culture of two or more of: a) a bacterium having 97% sequence identity to the 16S rRNA gene sequence of Akkermansia species—like strain GGCC_0220 (SEQ ID NO: 1) and genetic material encoding for synthesis of propionate, synthesis of 4-amino-butyrate (gamma-aminobutyric acid; GABA), and synthesis of indole or indole-containing secondary metabolites; b) a bacterium having 97% sequence identity to the 16S rRNA gene sequence of Bacteroides uniformis—like strain GGCC_0301 (SEQ ID NO: 2) and genetic material encoding for synthesis of propionate, synthesis of GABA, and synthesis of indole or indole-containing secondary metabolites; c) a bacterium having 97% sequence identity to the 16S rRNA gene sequence of Bacteroides xylanisolvens—like strain GGCC_0124 (SEQ ID NO: 3) and genetic material encoding for synthesis of propionate, synthesis of GABA, and synthesis of indole or indole-containing secondary metabolites; d) a bacterium having 99% sequence identity to the 16S rRNA gene sequence of Barnesiella species—like strain GGCC_0306 (SEQ ID NO: 4) and genetic material encoding for synthesis of propionate and synthesis of GABA; e) a bacterium having 97% sequence identity to the 16S rRNA gene sequence of Bitterella massiliensis—like strain GGCC_0305 (SEQ ID NO: 5) and genetic material encoding for 3α-hydroxy steroid dehydrogenase and 3β-hydroxy steroid dehydrogenase; f) a bacterium 97% sequence identity to the 16S rRNA gene sequence of Clostridium butyricum—like strain GGCC_0151 (SEQ ID NO: 6) and genetic material encoding for synthesis of butyrate; g) a bacterium having 97% sequence identity to the 16S rRNA gene sequence of Clostridium scindens—like strain GGCC_0168 (SEQ ID NO: 7) and genetic material encoding for bile acid 7-alpha-dehydratase activity; h) a bacterium having 97% sequence identity to the 16S rRNA gene sequence of Clostridium symbiosum—like strain GGCC_0272 (SEQ ID NO: 8) and genetic material encoding for synthesis of butyrate; i) a bacterium having 97% sequence identity to the 16S rRNA gene sequence of Eubacterium callanderi—like strain GGCC_0197 (SEQ ID NO: 9) and genetic material encoding for synthesis of butyrate, synthesis of GABA, and bile acid 7-alpha-dehydratase activity; j) a bacterium having 97% sequence identity to the 16S rRNA gene sequence of Extibacter species—like strain GGCC_0201 (SEQ ID NO: 10) and genetic material encoding for bile acid 7-alpha-dehydratase activity; and k) a bacterium having 97% sequence identity to the 16S rRNA gene sequence of Intestinimonas butyriciproducens—like strain GGCC_0179 (SEQ ID NO: 11) and genetic material encoding for synthesis of butyrate.

In the methods of the presently disclosed subject matter, the subject can be a human.

In the methods of the presently disclosed subject matter, the inflammatory disorder can include ulcerative colitis, Crohn's disease, Ankylosing Spondylitis, Plaque Psoriasis, Psoriatic Arthritis or Pouchitis. The composition can be administered in combination with one or both of a corticosteroid and one or a combination of the drugs listed in Table 3.

In the methods of the presently disclosed subject matter, the inflammatory disorder can include Alzheimer's and Non-Alzheimer's disease related dementia, Parkinson's disease, multiple sclerosis, Type-2 diabetes, obesity, hepatic steatosis, or aging related cardiovascular or neurologic conditions.

In the methods of the presently disclosed subject matter, the composition can be administered in combination with one or a combination of a food supplement or a prebiotic. The food supplement or prebiotic can include by way of nonlimiting example, one or more of almond skin, inulin, oligofructose, raffinose, lactulose, pectin, hemicellulose (such as xyloglucan and alpha-glucans), amylopectin, and resistant starch which are not decomposed in the upper gastrointestinal tract and promote the growth of intestinal microbes in the intestinal tract, as well as growth factors such as acetyl-Co A, biotin, beet molasses, and yeast extracts.

In one embodiment, a method is provided for monitoring systemic inflammation in a subject, including: measuring a level of one or a combination of a secondary metabolite biomarker in a stool sample of a subject, wherein the biomarker is selected from the group consisting of propionate, indole and its derivatives indole propionate (IPA) and indole acetate (IAA), and secondary bile acids deoxycholic acid (DCA) and lithocholic acid (LCA), wherein an increase in the level of the one or a combination of biomarkers is an indication of a decrease in systemic inflammation in the subject.

For example, the levels of the above-mentioned secondary metabolites in a patient's stool sample can be determined to create a baseline. The levels of one or a combination of these secondary metabolite biomarkers in subsequent stool samples are then indicative of efficacy of the intervention strategy and the progression of inflammation. In addition, monitoring the levels of one or a combination of these key metabolite biomarkers can be used to evaluate symptom-free long-term remission and to predict potential relapses.

In addition to use in validating the establishment and performance of live biotherapeutic products, such as the GUT-108 consortium, measuring the progression of the levels of one or a combination of these secondary metabolites in stool samples has general applicability to monitor the long-term remission of patients treated with common therapeutics for conditions involving chronic inflammation (e.g., corticosteroids, antibiotics, immunomodulators, REMICADE, HUMIRA, ENTYVIO, STELARA, TALTZ, XEUANZ, and biosimilars such as RENFLEXIS (Infliximab-abda; Samsung and marketing partner Merck). Therefore, in one embodiment, measuring the progression of the levels of one or a combination of these secondary metabolites in stool samples is provided as a non-invasive method to monitor the progression of inflammation and to validate intervention strategies for conditions involving chronic inflammation that are characterized by a microbiome component.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

Microbial Therapeutic Consortium for the Treatment of Chronic Inflammation

Bacterial strains were isolated from stool samples of healthy individuals. All manipulations of stool and bacteria were conducted under an anaerobic atmosphere comprised of 5% H₂, 10% CO₂ and 85% N₂ (v:v:v). Stool samples were freshly collected, and 10⁻⁴ and 10⁻⁵ dilutions (w:v) were prepared in 10 mM MgSO₄ solution that had been reduced under anaerobic conditions. Dilutions of stool samples were plated on reduced solid LYH-BHI medium (Brain-heart infusion medium supplemented with 0.5% yeast extract (Difco)), or solid Peptone Yeast Glucose broth medium (Anaerobe Systems). The plates were incubated at 37° C. under anaerobic conditions for 5 to 7 days. The plates were inspected daily for the appearance of bacterial colonies. Colonies with distinct morphologies were purified on the same medium they were isolated on. Once biologically pure cultures were obtained, their DNA was extracted, and the bacteria were subsequently identified by sequencing of their 16S rRNA gene. Using this approach, several hundred bacterial strains were isolated that had not been previously identified.

For functional characterization and to determine the presence of key functionalities, whole genome sequencing was performed. Bacterial genome sequencing libraries were generated using the ThruPLEX DNA-seq Kit (Rubicon Genomics). Individual strain libraries were combined in equimolar proportions in one pool and sequenced by 125 bp pair end read sequencing on the Illumina HiSeq2500. Onboard image processing and base calling were performed. The sequence data quality score (Q score) was used as a quality control metric with the specification that ≥80% of bases must have a Q score of ≥30. After trimming, sequencing reads were assembled with SPAdes (version 3.13.0) using default parameters and annotated using Prokka (version 1.14-dev) and the RAST server.

For deposition under the terms of the Budapest Treaty on the International Recognition of the Deposit of Microorganisms for the Purposes of Patent Procedure at the American Type Culture Collection (ATCC) in Manassas, Virginia, USA, 300 ml cultures of the 11 previously unidentified strains included in the GUT-108 consortium were prepared. All strains were grown in liquid LYH-BHI medium, except Bitterella massiliensis GGCC_0305 and Barnesiella sp. GGCC_0306, which were grown in liquid Peptone Yeast Glucose broth. Strains were cultivated at 37° C. under strict anaerobic conditions (H₂ (5%), CO₂ (10%) and N₂ (85%) v/v/v) for 7 days till reaching their stationary growth phase. The cells were harvested via centrifugation and the cell pellets were suspended in 30 ml of pre-reduced sterile Freezing Buffer, which contained 9 g NaCl and 150 ml glycerol in H₂O per liter (total volume). Per strain, 25 aliquots of 1 ml concentrated bacterial suspension were provided to ATCC.

Starting with a collection of over 300 previously unidentified bacterial strains isolated from stool samples of healthy individuals, an 11-strain consortium referred to as GUT-108 was rationally designed as described below (see TABLE 1). GUT-108 includes enhanced redundancy for synthesis of the therapeutic secondary bile acids LCA and DCA provided by strains Clostridium scindens—like GGCC_0168, Extibacter sp. GGCC_0201 and Eubacterium callanderi—like GGCC_0197; yersiniabactin synthesis for siderophore production provided by strain Clostridium symbiosum GGCC_0272; and functions antagonistic against opportunistic pathogens belonging to the Enterobacteriaceae provided by several strains.

More specifically, the 11-strain consortium GUT-108 is based on the presence of the key functionalities: synthesis of butyrate, propionate, indole and indole-containing secondary metabolites, GABA, siderophore synthesis or uptake of heterologous siderophores, and synthesis of antagonistic molecules, including bacteriocins, as well as deconjugation and conversion of bile salts into secondary bile acids and uptake of heterologous siderophore. The designated species of each of the strains is the closest relationship based on sequence homology to 16S rRNA gene sequence. Specifically, in GUT-108 comprises Akkermansia species—like strain GGCC_0220 (SEQ ID NO: 1), Bacteroides uniformis—like strain GGCC_0301 (SEQ ID NO: 2), Bacteroides xylanisolvens— like strain GGCC_0124 (SEQ ID NO: 3), Barnesiella species—like strain GGCC_0306 (SEQ ID NO: 4), Bitterella massiliensis—like strain GGCC_0305 (SEQ ID NO: 5), Clostridium butyricum—like strain GGCC_0151 (SEQ ID NO: 6), Clostridium scindens—like strain GGCC_0168 (SEQ ID NO: 7), Clostridium symbiosum—like strain GGCC_0272 (SEQ ID NO: 8), Eubacterium callanderi— like strain GGCC_0197 (SEQ ID NO: 9), Extibacter species—like strain GGCC_0201 (SEQ ID NO: 10), and Intestinimonas butyriciproducens—like strain GGCC_0179 (SEQ ID NO: 11). The genome annotation platform RAST was used to confirm the presence of the key functionalities in the strains (see TABLE 1) and their metabolic interdependencies based on auxotrophies (see TABLE 2).

Based on annotation and modeling it was concluded that Clostridium symbiosum—like strain GGCC_0272, which possesses a putative Yersiniabactin synthesis operon, can produce its own siderophore. Uptake of the heterologously produced siderophore aerobactin was identified for Bacteroides uniformis—like strain GGCC_0301, Bacteroides xylanisolvens—like strain GGCC_0124, and Barnesiella species—like strain GGCC_0306, while uptake of the heterologously produced siderophore enterobactin was identified for Barnesiella species—like strain GGCC_0306, Bitterella massiliensis—like strain GGCC_0305, Clostridium butyricum—like strain GGCC_0151, Clostridium symbiosum—like strain GGCC_0272, Eubacterium callanderi—like strain GGCC_0197, Extibacter species—like strain GGCC_0201, and Intestinimonas butyriciproducens—like strain GGCC_0179. The presence of these heterologous siderophore uptake systems can allow for these strains to compete against opportunistic pathogenic bacteria (including enterohemorrhagic, enteroaggregative, and adherent invasive Escherichia coli and Klebsiella species) that can thrive in the dysbiotic gut environment associated with IBD and other conditions of chronic inflammation that have a microbiome component associated with them.

Key functionalities for bile salt identified for the members of the eleven-strain GUT-108 consortium were confirmed using the genome annotation platform, RAST (Table 1). Based on the results it can be concluded that members of the eleven-strain rationally designed consortium for inflammation control cover a range of activities to deconjugate and subsequently modify bile salts into secondary bile acids. Through comparison of gene similarity with Clostridium scindens—like strain GGCC_0168, putative bile acid 7-alpha-dehydratase activity was identified for the strains Eubacterium callanderi—like strain GGCC_0197 and Extibacter species—like strain GGCC_0201. This result was unexpected given that this activity has not been previously reported for strains of Eubacterium callanderi or Extibacter species.

Key functionalities for bacteriocin and lantibiotic synthesis and resistance putatively identified for the members of the eleven-strain rationally designed consortium for inflammation control were confirmed using the genome annotation platforms RAST and antiSMASH (Table 1).

The strains were also evaluated for their putative synthesis of indole. Akkermansia species—like strain GGCC_0220, Bacteroides uniformis—like strain GGCC_0301, and Bacteroides xylanisolvens—like strain GGCC_0124 were found to possess a putative Tryptophanase (EC 4.1.99.1) gene that catalyzes the breakdown of tryptophan into indole, pyruvate and NH₃.

The strains were also evaluated for their putative synthesis of short chain fatty acids, especially propionate and butyrate. Propionate synthesis was identified for Akkermansia species—like strain GGCC_0220, Bacteroides uniformis—like strain GGCC_0301, Bacteroides xylanisolvens—like strain GGCC_0124, and Barnesiella species—like strain GGCC_0306, while butyrate synthesis was identified for Clostridium butyricum—like strain GGCC_0151, Clostridium symbiosum—like strain GGCC_0272, and Eubacterium callanderi— like strain GGCC_0197.

The strains were also evaluated for their putative synthesis of gamma-amino-butyric acid (GABA). Akkermansia species—like strain GGCC_0220, Bacteroides uniformis—like strain GGCC_0301, Bacteroides xylanisolvens—like strain GGCC_0124, Bamesiella species—like strain GGCC_0306, and Eubacterium callanderi—like strain GGCC_0197 were found to possess a putative Glutamate decarboxylase (EC 4.1.1.15) gene that catalyzes the breakdown of glutamate into GABA and CO₂.

Another advantageous feature of the microbial therapeutic consortium GUT-108 described herein is that its members by complementing each-others auxotrophies (Table 2) create a network of metabolic dependencies designed to promote stability of the consortium in the hostile dysbiotic environment of intestinal inflammation. To build the GUT-108 consortium model for metabolic interdependency, computational models were first built for the individual GUT-108 strains (Henry et al, 2010). All strain models required some degree of gap filling to ensure that they are capable of synthesizing or acquiring all the small molecule building blocks required to produce new biomass. This gap filling was performed in silico mimicking a specific growth condition; it is preferable to perform gap filling mimicking minimal medium composition. The initial in silico gap filling was therefore performed in glucose minimal medium. Thus, an auxotrophy analysis was performed to predict defined minimal media for each of the GUT-108 strains. In this analysis, the synthesis pathways for all amino acids, vitamins, and cofactors were computationally assigned and subsequently, as part of a quality control process, manually reviewed in a model-driven fashion to determine which pathways were likely incomplete for each genome. The output of this analysis revealed very substantial differences in auxotrophies across the GUT-108 strains (See Table 2).

### Example 2

### Evaluation of GUT-108 for the Treatment of Colitis

To evaluate the efficacy of the GUT-108 consortium to induce an anti-inflammatory IL-10 response, a study was performed using a validated animal model based on eGFP-reporter (Il10⁺/^{eGFP}) C57BL/6J mice, also referred to as VertX mice. In these mice, which have a wild-type IL-10 phenotype, induction of IL10 synthesis can be visualized by expression of the eGFP reporter. In addition, the ability of GUT-108 to become engrafted in the mouse intestinal tract was evaluated in two different germ-free mice models, the VertX reporter line and in ex-germ-free (sterile) Il10^{-/-} 129SvEv mice.

Further, to evaluate the efficacy of the GUT-108 consortium to treat IBD, a study was performed using a validated animal model based on the Il10^{-/-} 129SvEv mice where colitis was induced by inoculating the animals with 200 μl/mouse of diluted human donor stool (1:100 stool dilution with anaerobic PBS). Inoculation of Il10^{-/-} mice with the human donor stool results after 2 weeks in a moderate to severe chronic, bacterial antigen-specific Th1/Th17 driven inflammatory immune response. The design of the IBD experiment is provided below:
1. Gavage with human donor stool. As stool material from the same donor has been previously used, it has a known timeline for causing disease and is serving as a positive control;
2. GUT-108 consortium+human donor stool in a therapeutic protocol. The GUT-108 consortium was applied 2 weeks after administration of the human donor stool in a therapeutic protocol (delayed therapy after onset of disease).

The following experimental protocols were used:

Microbiology techniques. All bacterial strains were grown on LYH-BHI medium (Brain-heart infusion medium supplemented with 0.5% yeast extract (Difco)), except Bitterella massiliensis GGCC_0305 and Barnesiella sp. GGCC_0306 which were grown on Peptone Yeast Glucose broth (Anaerobe Systems). Strains were cultivated at 37° C. under strict anaerobic conditions (H₂ (5%), CO₂ (10%) and N₂ (85%) v/v/v). After growth, cultures were concentrated at 10⁺⁹ cfu/ml in phosphate solution (PBS) buffer. Cultures were tested for contamination by streaking them on LB medium, followed by incubation at 37° C. under aerobic conditions. A culture was considered pure if no growth was observed. As part of the quality control process, the 16S rRNA gene of each culture was amplified and sequenced.

DNA sequencing. Bacterial genome sequencing libraries were generated using the ThruPLEX DNA-seq Kit (Rubicon Genomics). Individual strain libraries were combined in equimolar proportions in one pool and sequenced by 125 bp pair end read sequencing on the Illumina HiSeq2500. Onboard image processing and base calling were performed. The sequence data quality score (Q score) was used as a quality control metric with the specification that ≥80% of bases must have a Q score of ≥30. After trimming, sequencing reads we assembled with SPAdes (version 3.13.0) and annotated using Prokka (version 1.14-dev) and the RAST server. Fecal DNA was extracted with AllPrep PowerViral DNA/RNA Kit (Qiagen). Sequence libraries were constructed using the KAPA Hyper DNA library prep. DNA libraries were multiplexed and loaded on an Illumina HiSeq4000 instrument per manufacturer's instructions. Sequencing was performed using a 2×150 paired-end configuration; image analysis and base calling were conducted by the HiSeq Control Software on the HiSeq instrument. Sequencing reads were trimmed, and mouse reads were filtered out using Trimmomatic (version v0.39). Species abundance was determined with Kaiju (version v1.7.2).

Quantitative (RT)-PCR of bacterial DNA. Q-PCR using strain specific primers against the single copy RpoB gene were used to quantify the composition of GUT-108 after gavage in gnotobiotic mice. The genomic DNA was extracted from fecal or culture media using AllPrep PowerViral DNA/RNA Kit (Qiagen). Q-PCR were performed with QuantStudio3 (Thermo Fisher Scientific, PA, USA) using SYBR No-ROX reagents (Bioline, TN, USA) with the following PCR setting: 95° C., 3 min; 95° C., 5 sec; 40 cycles of (60° C., 10 sec; 72° C., 20 sec); melting curve analysis: 95° C., 15 sec; 60° C., 15 sec; 95° C., 15 sec. The data were created by comparative Ct method (2^{-Ct}). Melting curve analysis confirmed the presence of single products with expected melting temperatures.

Mice. 8-12 week-age Germ-free (GF) 129SvEv background IL-10-deficient mice (Il10^{-/-}) were obtained from University of North Carolina (UNC) National Gnotobiotic Rodent Resource Center. Il10-eGFP-reporter (Il10⁺/^{eGFP}) mice based on C57BL/6J background mice were originally provided by Dr. C. L. Karp (Global Health, Bill & Melinda Gates Foundation, USA) and house-raised at the UNC National Gnotobiotic Rodent Resource Center. Mice chow is TD2020. Germ-free and gnotobiotic mice were maintained in positive-pressure isolators and housed in separate polycarbonate cages at constant room temperature (22° C.±10%), air humidity (50%±20%), and a light/dark cycle of 12 h. Mice had free access to food and water. Standard mouse chow (TD2020SX; Teklad Diets, Madison, WI) was sterilized by irradiation at 25 kGy. GF mice were colonized with human feces by oral gavage and 3-4 mice/cage were housed within gnotobiotic Trexler isolators (EER/GUT-103 experiments and gnotobiotic GUT-108 experiments) or in sterilized cages with autoclaved food and water (humanized/GUT-108 experiments).

Inoculation. Per condition 11 mice were used. For treatment human donor stool, 200 μl of diluted human donor stool (Donor-Y, 1:100 dilution with anaerobic PBS) was applied by oral gavage on day 1 to 129SvEv background IL-10-deficient (Il10^{-/-}) mice. For application of GUT-108, 300 μl resuspended GUT-108 strain mixture in anaerobic PBS was applied per mouse by oral gavage. All GUT-108 consortium strains were grown individually, subsequently mixed, and provided at a dose of 2.0×10⁺⁷ cfu per strain in a total volume of 300 The strain mixture was provided four times via oral gavage on days 15, 17, 22 and 25 (129SvEv background IL-10-deficient (Il10^{-/-}) mice model) or on days 1, 3, 8 and 11 (eGFP-reporter (Il10⁺/^{eGFP}) mice).

Fecal collection and consistency score. Fresh feces (2-5 pieces/mouse) were collected and immediately snap-frozen on dry ice and stored at −80° C. Stool consistency was scored by validated scoring system (Fitzpatrick et al, 2011).

Metabolites Analysis. The metabolite extraction and measurement methods were performed as described previously (Zheng et al, 2013). All samples were analyzed in an Agilent 7890A gas chromatography system with an Agilent 5975C inert XL El/CI mass spectrometric detector (MSD, Agilent Technologies, Santa Clara, CA). An HP-5 ms capillary column (Agilent J & W Scientific, Folsom, CA) was used to separate samples. 1 μL derivatives was injected in split mode (10:1), with a solvent delay time 2.2 min. The GC oven temperature gradient was programmed as following: initial oven temperature was set at 50° C., held for 2 min, and then increased temperature up to 70° C. by a rate of 10° C./min, and increased to 85° C. by a rate of 3° C./min, and then to 110° C. by a rate of 5° C./min, to 290° C. by a rate of ° C./min, and finally held at 290° C. for 8 min. The carrier gas used Helium with a 1 mL/min constant flow rate. The mass spectrometer operated at a full scan mode with m/z range from 30 to 600.

Cell isolation. Mesenteric lymph nodes (MLNs) were mechanically dissociated in RPMI1640 (Gibco/Life Technologies, CA, USA) containing 5% heat-inactivated fetal bovine serum (FBS) (Millipore-Sigma, MA, USA) and 100 U/ml penicillin-streptomycin (Gibco/Life Technologies). Red blood cells (RBCs) in spleen samples were lysed with red blood cell lysing buffer (Sigma-Aldrich). Cell preparations were filtrated through 70-μm nylon mesh (Fisher Scientific, PA, USA) to achieve single-cell suspensions. Colonic tissues were opened longitudinally, washed twice with 1×PBS, cut into 1 cm pieces and incubated with stirrer for 250 r.p.m. in HBSS (Corning) medium containing 2.5% FBS (Sigma-Aldrich), 1% penicillin-streptomycin (Gibco), 4 mM EDTA (Corning) and 10 mM dithiothreitol (Sigma) for 20 min at 37° C. to remove the epithelial layer. Denuded tissue samples were washed twice with HBSS containing 2.5% FBS and 1% penicillin-streptomycin and incubated with stirrer for 450 r.p.m. in HBSS containing 2.5% FBS, 0.5 mg/ml of collagenase (Sigma) for 30 min at 37° C. Cell preparations were filtrated through 100-μm nylon mesh to achieve single-cell suspensions. Lamina propria cells were purified using a 40-70% discontinuous Percoll gradient (GE Healthcare, 2,000 r.p.m., 20 min, room temperature) and washed with HBSS.

Staining cells for flow cytometry analysis. Single cells were stained for 20 min at 4° C. after FcγRII/III blocking with anti-CD16/CD32 monoclonal antibody. For intracellular staining, cells were re-stimulated with 50 ng/ml phorbol 12-myristate 13-acetate (PMA, Sigma) and 500 ng/ml ionomycin (Sigma) for 4 h at room temperature with 1 μl/ml protein transport inhibitor (GolgiStop, BD) during the last 3 h. After washing, cells were first surface stained, then fixed for 5 min at 37° C. using PBS containing 4% paraformaldehyde (Electron Microscopy Sciences) and 0.01% Tween20 (Fisher Scientific), permed using PBS containing 0.1% Triton X-100 (MP Biomedicals), 0.5% BSA (Sigma), 2 mM EDTA (Corning) for 45 min at room temperature, and stained overnight with indicated antibodies. Singlet live CD45⁺ cells were analyzed by FlowJo software version 10 (FlowJo, OR, USA) with the following gate strategy: B cell (B220⁺CD19⁺), CD4⁺ T cell (TCRβ⁺CD3⁺CD4⁺CD8^{neg}), macrophage (TCRβ^{neg}CD11b⁺CD64⁺) and dendritic cell (TCRβ^{neg}CD64^{neg}MHCII⁺CD11c⁺). For GFP-positive gate, GFP-negative colonic lamina propria cells from C57BL/6 wild-type mice were stained with all antibodies used in the experiment as a fluorescence-minus-one control.

Fecal/bacterial lysate. For in vitro cell stimulation with bacterial products, bacterial lysate was prepared from human donor stool or cultured GUT-108. Fecal or bacterial pellets were disrupted with glass microbeads (BioSpec Products, OK, USA) in MD solution containing 0.1 M magnesium chloride (Sigma-Aldrich, MO, USA), 0.1 mg/ml DNase I (Worthington Enzymes) by using a bead beater (TeSeE Precess48 homogenizer, 2 cycles of 6500 speed×45 sec, Bio-Rad Laboratories, CA, USA) and were centrifuged at 10,000 r.p.m. for 15 min at 4° C. The resulting supernatants were filtrated through filter (Genesee Scientific, CA, USA). Sterility of lysates was verified by YCFA agar culture at 37° C. for 5 days aerobically and anaerobically (Whitley MG500 workstation, N2:H2:CO2=80:10:10, Don Whitley Scientific, West Yorkshire, UK). Protein concentrations were measured according to the manufacturer's instructions (Bio-Rad Laboratories).

MLN culture with lysates. 1×10⁶ MLN cells were cultured in RPMI1640 containing 10% FBS, 100 U/ml penicillin-streptomycin, 55 μM 2-mercaptoethanol and 1 mM sodium pyruvate in 96-well round-bottom plates (Costar, MA, USA) for 24 hours at 37° C. with 5% CO₂ with human donor stool lysates or GUT-108 lysates. Following cell cultures, supernatants were collected for measurements of cytokines by ELISA, while cells were analyzed by flow cytometry.

Gut culture (tissue fragment culture). Colonic tissue fragment cultures were prepared from the large intestine and the cecum. Colonic tissues were thoroughly irrigated with PBS, shaken at room temperature in RPMI media containing 50 μg/ml gentamicin (Sigma) for 30 min at 250 r.p.m., cut into 1 cm fragments. Colonic tissue fragments were dried up with paper, weighed and distributed (50 mg/well) into 24-well plates (Costar) and incubated in 1 ml of RPMI media supplemented with 5% FBS, μg/ml gentamicin and 1% antibiotic/antimycotic (penicillin/streptomycin/amphotericin B; GIBCO) for h at 37° C. in CO₂ incubator. Supernatants were collected and stored at −20° C. before use for cytokine quantification. The data were normalized by tissue weight.

Histology and scoring. The intestines were fixed in 10% neutral-buffered formalin. Paraffin-embedded sections (5 μm) were stained with hematoxylin and eosin. Ileal-end, cecum, proximal-, distal-colon and rectum were quantitated in a blinded fashion by well validated histological scoring system (Liu et al, 2011).

Statistical analysis. GraphPad Prism8 software was used for statistical analysis.

The following results were obtained:

GUT-108 is successfully established in gnotobiotic mice models. GUT-108 gavaged into two different germ-free mice models, Il-10^{+/eGFP} VertX reporter and Il10^{-/-} mice, established engraftment of all strains. No significant differences were observed between ratios of GUT-108 strains in the Il10⁺/^{eGFP} VertX reporter (FIG. 1A) andIl10^{-/-} mice (FIG. 1B).

Visual evaluation of the degree of tissue inflammation from representative mice cecum tissue samples showed that colonization with GUT-108 did not induce inflammation. FIGS. 2A and 2B show that treatment with GUT-108 did not induce colitis in the susceptible germ-free Il10^{-/-} mice.

Importantly, GUT-108 colonization promoted a healthy gut microbiome with key functionalities for SCFA, especially synthesis of acetate and propionate, and IAA synthesis being restored (FIG. 3A-3D).

Four GUT-108 strains, Clostridium butyricum GGCC_0151, Intestinimonas butyriciproducens GGCC_0179, Bitterella massiliensis GGCC_0305 and Barnesiella sp. GGCC_0306 were established at levels below 1% (FIG. 1). While the significance of the relatively low abundance of these four strains is unclear, the key metabolic phenotypes of these four strains were likely also provided by other members of the GUT-108 consortium due to the designed redundancies in metabolic interdependencies and therapeutic functions in GUT-108 (see FIG. 3A-3D).

The ability of the GUT-108 consortium to stimulate regulatory (protective) IL-10-mediated immune response was determined by comparing the type and levels of IL-10 producing immune cells in Il10⁺/^{eGFP} VertX reporter mice as described above. Mice were either treated by gavage with PBS buffer solution (negative control) or GUT-108. The results are described below in FIG. 4 and FIGS. 5A-5H. Two weeks after being introduced, GUT-108 stimulated increased numbers of colonic lamina propria (LP) IL-10-producing CD4⁺ T cells, B cells and dendritic cells (DC) and increased numbers and percentages of regulatory T cells, including inducible Tregs (IL-10⁺ RoRγT⁺ FoxP3⁺ CD4⁺ cells), IL-10⁺ Tregs and FoxP3^{neg} IL-10⁺ T cells (TR₁) (FIG. 5A-5H). The increased levels of various IL10 synthesizing immune cell types points to a systemic anti-inflammatory response induced by GUT-108 that has broad applicability for treatment of a range of conditions characterized by a disease pathology including chronic inflammation of the gastrointestinal tract, such as Inflammatory Bowel Diseases (IBD), Irritable Bowel Syndrome (IBS), graft versus host disease, Parkinson's disease, ALS, systemic lupus erythematosus, asthma, food allergies, fatty liver, hepatic encephalopathy, type-2 diabetes, metabolic syndrome and obesity, psoriasis, and well as aging related conditions caused by chronic inflammation, each of which have been shown to have a microbially-mediated inflammatory component.

The ability of the GUT-108 consortium to therapeutically treat moderate to chronic, immune-mediated experimental colitis as a model of IBD was determined by comparing the level of inflammation in ex-germ-free IL-10−/− mice selectively colonized with human donor stool (positive control for the onset of colitis), and human donor stool plus GUT-108 (therapeutic protocol with GUT-108 applied 2 weeks after the onset of colitis induced by human stool application) as described herein above. The results are described below.

Therapeutic application of GUT-108 was tested in a clinically relevant experimental colitis model using gnotobiotic Il10^{-/-} mice (129SvEv background) humanized with fecal microbiota derived from a healthy human subject. Experimental colitis was induced by inoculating the mice with a human stool previously verified to induce aggressive colitis in gnotobiotic Il10^{-/-} mice. Therapeutic application of GUT-108 beginning 2 weeks after human fecal colonization resulted in changes in community composition. Metagenome analysis showed that therapeutic application of GUT-108 resulted in engraftment of all GUT-108 strains except Clostridium scindens GGCC_0168, restoring a healthy gut microbiome that promoted the abundance of beneficial resident Clostridium (Clusters IV and XIVa) strains (not part of the GUT 108 consortium) and decreased by 90% the abundance of Enterobacteriaceae from 5% to below 0.5%, including Klebsiella pneumoniae, Salmonella enterica, Escherichia coli and Shigella species (FIG. 6). An eight-fold decrease in the levels of the pathogenic Clostridium perfringens was also observed after treatment with GUT-108 (FIG. 6).

In the dysbiosic gut microbiome of Il10^{-/-} mice colonized with human fecal microbiota, beneficial Lachnospiraceae and Ruminococcaceae family members are decreased while opportunistic pathogens belonging to the Enterobacteriaceae are increased compared to the donor fecal community, as also reported for Crohn's disease patients (Vila et al, 2018). Therapeutic application of GUT-108 reduced levels of colitogenic Enterobacteriaceae and increased beneficial resident Clostridium (Clusters IV and XIVa) species, especially Lachnospiraceae including Dorea species and Lachnoclostridium species that are not GUT-108 constituents (FIG. 6).

The progression of inflammation based on the levels of lipocalin 2 secreted in the stool of selectively colonized gnotobiotic 129 Il10^{-/-} knock-out mice was evaluated in function of time after gavage with human fecal material and human fecal material plus the GUT-108 consortium in a therapeutic protocol (2 week delay in administering GUT-108 after gavage with human fecal material). Lipocalin 2 is a rapid indicator for distal intestinal inflammation; the higher the levels of lipocalin in the stool, the more severe the degree of gut inflammation. The results are presented in FIG. 7. Two weeks after the first gavage, an elevated level of lipocalin 2 in the stool samples was observed (compared to basal lipocalin 2 levels of approximately 3 ng/g fecal material) due to an immunological reaction and the onset of inflammation caused by the introduction of human fecal material in the gut of Il10^{-/-} knock-out mice that were previously germ free. After three and four weeks, the average lipocalin 2 levels in the stool of Il10^{-/-} knock-out mice treated with human fecal material were 723 ng/g and 3675 ng/g, respectively, this in contrast to the lipocalin 2 levels in the stool of Il10^{-/-} knock-out mice that two weeks after the initial gavage with human fecal material had received GUT-108, which were 91 ng/g and 531 ng/g (after 2 and 3 weeks, respectively; see FIG. 7). This indicates that the application of GUT-108 starting two weeks after the initial gavage with human fecal material resulted in a decrease of the level of colonic inflammation as compared to the control, showing the therapeutic effect of the GUT-108 consortium to treat chronic experimental colitis.

The therapeutic effect of GUT-108 was further confirmed by determining the stool score, which is an index for stool consistency, where a higher score is indicative for inflammation. The stool score was determined according to Gut Pathogens 2011; 3:16, as follows: Score 0: normal; Score 1: loose stool; Score 2: loose/some diarrhea; Score 3: diarrhea; and Score 4: severe watery diarrhea. The results are presented in FIGS. 8A and 8B. At week 4, Il10^{-/-} knock-out mice treated with human fecal material had a stool consistency score of 1.27, while Il10^{-/-} knock-out mice that two weeks after the initial gavage with human fecal material had received GUT-108 had a stool consistency score of 0.5. FIGS. 9A and 9B show the effectiveness of GUT-108 in reducing the induced inflammation in the cecum tissue relative to the PBS control.

The images in FIGS. 2A and 2B show that compared to PBS, four applications over a two-week period of GUT-108 via gavage to germ-free Il10^{-/-} mice did not result in the onset of inflammation in the cecum. However, gavage with human stool resulted in the onset of moderate to severe colitis (FIG. 9A). Compared to treatment with PBS, therapeutic treatment with GUT-108 (four applications over a two-week period after the onset of colitis induced by gavage with human fecal material) resulted in a significant decrease in the level of inflammation (FIG. 9B).

The degree of colitis was further examined by histological scoring (Sellon et al, 1998). Mice were killed four weeks after initial gavage with human fecal material. At necropsy, sections of colon (proximal, transverse, and distal) and cecum were fixed in 10% neutral buffered formalin. Duodenal and gastric tissue samples were taken from representative animals. The fixed tissue was embedded in paraffin and stained with H&E. The severity of inflammation was assessed blindly by a single individual and confirmed by an independent observer using a well-validated scale. Histological scores (0 to 12) were based on the degree of lamina propria and submucosal mononuclear cellular infiltration, crypt hyperplasia, goblet cell depletion, and architectural distortion. The results, presented in FIG. 10, show a statistically significant decrease in blinded histology score for the Il10^{-/-} mice that received GUT-108 two weeks after the application of human stool, as compared to Il10^{-/-} mice that didn't receive GUT-108. This further confirms that the application of GUT-108 two weeks after the initial gavage with human stool resulted in a reversal of established inflammation, showing the therapeutic effect of the GUT-108 consortium to treat experimental colitis.

As described in Example 1, the microbial composition of GUT-108 was designed to restore gut microbiome functionality. In order to confirm that the therapeutic application of GUT-108 resulted in the restoration of dysbiosis of gut microbiome functionality, levels of key therapeutic metabolites with anti-inflammatory activities (SCFA, indole and derivatives) or antagonistic activities (secondary bile acids) against pathogenic microorganisms were analyzed in the stool samples of Il10^{-/-} mice inoculated with human fecal material. Compared to treatment with PBS, therapeutic application with GUT-108 resulted in increased microbial synthesis of specific secondary metabolites with beneficial anti-inflammatory and antagonistic properties. The results are presented in FIGS. 11-13. Overall, levels of specific secondary metabolites encoded by the GUT-108 strains could be linked to restoration of functionality of the gut microbiome. Therefore, determining the levels of these metabolites in stool samples can be used as non-invasive biomarkers to monitor patients for the progression of inflammation and to validate the intervention strategies towards successful remission.

Butyrate synthesis and propionate synthesis have been described as important for modulation of the immune response. Unexpectedly, increased level of propionate (FIG. 11A) but not butyrate synthesis (FIG. 11B) was observed in mice that received therapeutic application of GUT-108 two weeks after gavage with human fecal material. Given these unexpected results, and without being limited to any specific mechanism of action, an explanation is provided in that propionate is a primary nutrient source for colonic epithelial cells, propionate is important for T cell induction, and increased levels of propionate can contribute to gut epithelial healing. Thus, increased levels of propionate may positively affect increased mucosal permeability that contributes to the severity of inflammatory disorders including ulcerative colitis, Crohn's disease, Ankylosing Spondylitis, Plaque Psoriasis, Psoriatic Arthritis and Pouchitis. The results show the importance of including non-Clostridium, propionate synthesizing strains in the GUT-108 composition.

Importantly, under pro-inflammatory conditions including increased mucosal permeability, butyrate synthesis can be based on the fermentation of amino acids, such as lysine, a process that contributes to inflammation associated with mucosal permeability. As such, compared to monitoring propionate levels, butyrate levels can be considered as a less reliable biomarker for monitoring gut microbiome dysbiosis and progression of inflammation linked to a broad spectrum of disease conditions with a gut microbiome component.

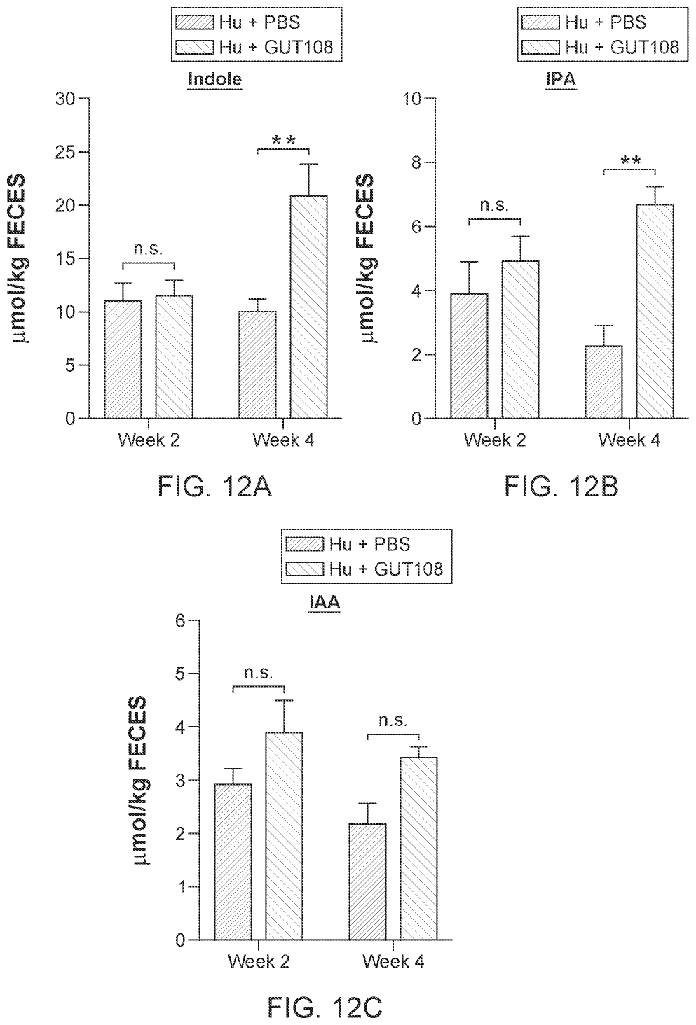

Increased synthesis of indole and its derivatives indole propionate (IPA) and indole acetate (IAA) was observed in mice humanized with a fecal transplant that received therapeutic application of GUT-108 (FIGS. 12A-12C). Especially IPA is considered a potent AhR pathway agonist that is critical in controlling epithelial barrier integrity. Thus, the increased synthesis of indole, IPA and IAA as the result of the therapeutic application of GUT-108 can positively contribute to gut epithelial healing, thus positively affecting the severity of inflammatory disorder including ulcerative colitis, Crohn's disease, Ankylosing Spondylitis, Plaque Psoriasis, Psoriatic Arthritis and Pouchitis. The results also show the importance of including non-Clostridium, indole synthesizing strains in the GUT-108 composition. The evolution of the levels of indole and its derivatives IPA and IAA provides a non-invasive biomarker to monitor the progression of inflammation and to validate intervention strategies that aim at addressing the increased mucosal permeability by restoring normal function to a dysbiotic gut microbiome.

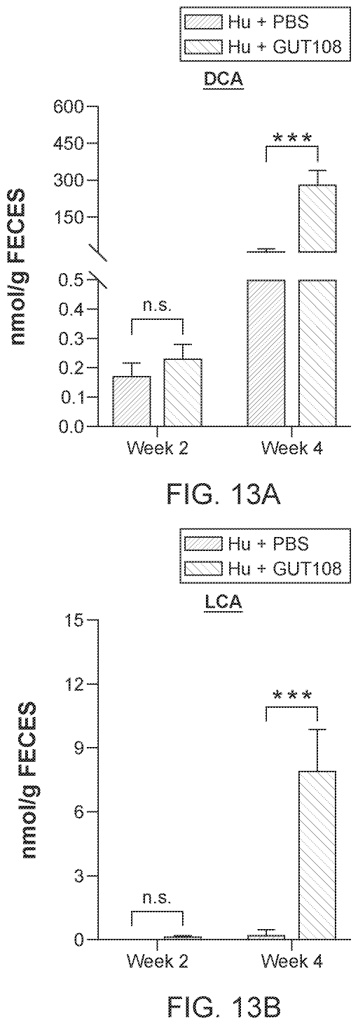

Metabolite analysis confirmed that the complex multi-strain pathway for the conversion of bile salts into therapeutic secondary bile acids did become engrafted and was functional in a humanized mouse model. This is shown for deoxycholic acid (FIG. 13A) and lithocholic acid (FIG. 13B), both of which have important therapeutic effects on health and infection control. When applied to Il10^{-/-} mice humanized with a fecal transplant, all GUT-108 strains except Clostridium scindens GGCC_0168 became established (FIG. 6). Clostridium scindens has been previously described as one of the essential strains necessary to convert primary bile acids into LCA and DCA. However, despite the absence of this strain, the established functional multi-strain network produced secondary bile acids, with Extibacter sp. GGCC_0201 providing the 7α-dehydratase activity required to convert CA and CDCA into the therapeutic secondary bile acids DCA and LCA, respectively. Normalizing the intestinal bile acid profile can restore intestinal epithelial stem cell function, and increase colonic RORγ+ Treg cell counts that ameliorate host susceptibility to colitis, while LCA stimulate differentiation to Tregs and inhibit Th17 cells consistent with GUT-108's ability to restore secondary bile acid metabolism (FIGS. 13A-13B) and activate inducible IL10+ RORγ FoxP3+ CD4+ Treg cells (FIGS. 5E, 5G). Lithocholic acid has also been reported to have an anti-aging effect due to its effect on mitochondrial lipid composition and energy processes, while deoxycholic acid is a strong antimicrobial with potent mode of action against microbial infections by pathogenic bacteria, including Clostridium perfringens and Clostridium difficile. The evolution of the levels of secondary bile acids, especially deoxycholic acid and lithocholic acid, provides a non-invasive biomarker to monitor and validate intervention strategies that aim at restoring a dysbiotic gut microbiome and to control infections by (opportunistic) pathogenic bacteria that thrive in the dysbiotic gut environment, including Clostridium perfringens and Clostridium difficile. The observed eight-fold decrease observed for the levels Clostridium perfringens in the mice treated with GUT-108 (FIG. 6) support the importance of increased levels of deoxycholic acid for pathogen control, including pathogenic Clostridium species such as, for example, Clostridium perfringens and Clostridium difficile. Overall, the levels of secondary metabolites in stool samples, more specifically the levels of one or a combination of propionate, indole and its derivatives indole propionate (IPA) and indole acetate (IAA), and secondary bile acids deoxycholic acid (DCA) and lithocholic acid (LCA), can be used as non-invasive biomarkers to monitor the progression of inflammation and to validate intervention strategies, including long-term remission of patients treated with common therapeutics for conditions involving chronic inflammation, including the therapeutics mentioned in Table 3.

The spontaneous secretion levels of IL-12p40 and IFNγ are very good indicators of the level of immune activation and inflammation of the colonic tissue. To determine spontaneous secretion levels of IL-12p40 and IFNγ, cultures of colon tissue fragments were prepared and cultured in 1 ml of complete medium containing antibiotics and an antimycotic agent. The cultures were incubated at 37° for 18 h with no stimulation. Culture supernatants were collected and stored at −20° C. until being assayed. IFNγ and IL-12p40 were measured by enzyme-linked immunosorbent assay (ELISA) with commercially available antibodies, similar to the IL-12p40 assay described by Sellon et al, (1998).

The results of this test are presented in FIGS. 14A and 14B and show that inoculation of germ-free strain 129 Il10^{-/-} knock-out mice with the human stool results in IL-12p40 and IFN-γ levels of 38 pg/mg colon tissue and 0.40 pg/mg colon tissue, respectively. After 2 weeks, therapeutic application of the GUT-108 consortium to strain 129 Il10^{-/-} knock-out mice that have an established human microbiome community and resulting inflammation in their gut results in a significant decrease in IL-12p40 (25 pg/mg colon tissue, compared to 38 pg/mg colon tissue) and IFNγ synthesis (0.32 pg/mg colon tissue, compared to 0.40 pg/mg colon tissue). These results further confirm the therapeutic effect of the GUT-108 consortium to treat chronic, immune-modulated colitis.

RT-Q-PCR was used to determine the therapeutic effect of GUT-108 on the levels of expression of the biosynthesis genes for the pro-inflammatory cytokines IL-1b, IL-6, IL-12b, IL-17α, IFNγ, IL-13, and TNFα, and the homeostatic cytokine IL-15 in the lamina propria of the colon of Il10^{-/-} knock-out mice humanized with a fecal transplant. Colonic lamina propria cells were isolated from mice that had either been inoculated with human donor stool or human donor stool plus GUT-108 in a therapeutic protocol, and total messenger RNA (mRNA) was isolated. Subsequently, mRNA was converted into cDNA, which was subsequently used in a Quantitative PCR (RT-Q-PCR) protocol to estimate levels of gene expression using gene specific primers. The results are presented in FIGS. 15A-15G and confirm the therapeutic effect of GUT-108, as illustrated by the significant decrease in levels of expression of the biosynthesis genes for the pro-inflammatory cytokines IL-1b, IL-12b, IL-17α, IFNγ, IL-13, TNFα; IL-6 synthesis was also reduced by approximately 60% (results not shown). In contrast, the level of expression of the biosynthesis gene for the homeostatic cytokine IL-15 was found to be significantly increased.

Interestingly, GUT-108 treatment increased expression of IL-15 mRNA (FIG. 15G), a homeostatic cytokine that controls T cell inflammatory responses, in gut tissue of Il10^{-/-} knock-out mice humanized with a fecal transplant. IL-15 is a homeostatic cytokine for T cells that controls inflammatory immune responses. Exogenous IL-15 treatment was found to result in decreased IL-17α expression by TH17 cells in vitro because of STATS enrichment at the IL-17 locus (Pandiyan et al, 2012). This is consistent with our observation of increased gene expression levels for IL-15 synthesis (FIG. 15G) and decreased gene expression levels for IL-17α synthesis (FIG. 15C) by colonic lamina propria cell.

Statistically significant decreased gene expression levels were also observed for the synthesis of the pro-inflammatory cytokines IL-1b, IL-12b, IFNγ, and TNFα (FIGS. 15A, 1513, 15D and 15F) and IL-6 (results not shown). In addition, decreased gene expression levels were observed for the synthesis of the pro-inflammatory cytokine IL-13 (FIG. 15F). These results further confirm that the therapeutic application of GUT-108 two weeks after the initial gavage with human stool resulted in a reversal of established inflammation, showing the therapeutic effect of the GUT-108 consortium to treat experimental colitis.

In addition to a decrease in the synthesis of proinflammatory cytokines, increased colonic expression levels of genes for receptors and pathways implicated in mucosal healing was observed by Q-PCR in gut tissue of Il10^{-/-} knock-out mice humanized with a fecal transplant (FIGS. 16A-16G).

Several protective pathway components that are decreased in IBD were upregulated in gut tissue of Il10^{-/-} knock-out mice humanized with a fecal transplant two weeks after application of GUT-108, further supporting its therapeutic role and providing mechanisms for protection (FIGS. 16A-16G). Expression of the Ahr pathway genes including the aryl hydrocarbon receptor (Ahr), the aryl hydrocarbon receptor repressor (AhrR) and the Cytochrome P450 Family 1 Subfamily A Member 1 (Cyp1A1) gene (Neavin et al, 2018) were significantly upregulated. Protective pathways with increased gene expression levels included the defensins DefCR1 and DefA, whose altered production is suggested to be integrally involved in IBD pathogenesis (Ramasundara et al, 2009), and the aldehyde dehydrogenases Aldh1A1 and Aldh1A2, whose expression is reduced in colonic macrophage and DC subsets of patients with ulcerative colitis regardless of inflammation (Magnusson et al, 2016). Increased Aldh1A1 and Aldh1A2 expression has been associated with increased activity of the retinoic acid pathway, which is critical in regulating Wnt/β-catenin signaling. Studies indicate that reduced activity of the retinoic acid pathway relate to tumor development and cellular migration. Several cellular pathways, including Wnt/β-catenin signaling pathway, are related to cancer metastasis, and many reports have suggested that exaggerated Wnt signaling can lead to cancer initiation and progression in a wide range of human tissues, including colon cancer, which has higher occurrence in IBD patients compared to healthy individuals. Therefore, increased expression of Aldh1A1 and Aldh1A2 as part of the retinoic acid pathway by GUT-108 has the potential to lower the risk of colorectal cancer in IBD patients.

Increased intestinal bacterial metabolism of tryptophan, especially indole and its derivatives IAA and IPA as observed in stool of Il10^{-/-} knock-out mice humanized with a fecal transplant two weeks after the therapeutic application of GUT-108 (FIGS. 12A-12C), activates the Ahr pathway. AHR acts as a sensor of the microbiota community and, through its established role of a modulating immune functions, maintains host-microbe homeostasis. IPA is also a pregnane X receptor (PXR) agonist mediating its responses through TLR4. GUT-108 therapy increased both IAA and IPA levels in stool (FIGS. 12B and 12C) and colonic Ahr gene expression (FIG. 16A). AHR is a critical mediator of anti-inflammatory responses to infection by bacterial pathogens and of the differentiation and function of immune cells including T cells, innate lymphoid cells, macrophages and DC. AHR promotes the expression of the anti-inflammatory cytokine IL-10 and inhibits macrophage apoptosis, decreases the expression of inflammatory cytokines (IL-6 and TNF-α) and inhibits activation of NF-κB. Therefore, the Ahr pathway is critical to protect from excessive inflammatory cytokine expression and septic shock. In addition, Ahr pathway activation protects the mucosa during inflammation.

Upregulation of Ahr has been associated with a positive outcome of various aging related conditions via its effect on various targets, including P-glycoprotein expression, fibroblast growth factor, tight junction proteins in the blood brain barrier, and the differentiation and function of immune cells, including T cells, macrophages and dendritic cells. Ahr activation via the gut microbiome will activate these targets, which has been described to have a positive effect on the development of Alzheimer's and Non-Alzheimer's disease related dementia, Parkinson's disease, multiple sclerosis, Type-2 diabetes, obesity, hepatic steatosis, and aging related cardiovascular and neurologic conditions, all of which have an underlying gut microbiome component. Therefore, in one embodiment, GUT-108 is provided as a therapeutic agent for the treatment of these conditions.

The anti-inflammatory effect of GUT-108 was also evaluated by determining the populations of CD4+ T cells in the lamina propria of the colon that synthesized the pro-inflammatory cytokines IL-17α and IFNγ. The results are presented in FIGS. 17A-17C and show a significant decrease of IFN-γ+, IL-17α+ and IFN-γ+ IL-17α+ synthesizing CD4+ T cells in the tissue of the colonic lamina propria of mice that had received GUT-108 in a therapeutic protocol after induction of moderate to severe colitis with human fecal material, as compared to mice that received the PBS placebo control. These results are in line with the decreased gene expression levels of genes for the synthesis of pro-inflammatory cytokines (FIGS. 15A-15F) in gut tissue of Il10^{-/-} knock-out mice humanized with a fecal transplant that receive therapeutic application of GUT-108, further confirm the anti-inflammatory effect of GUT-108 to treat experimental colitis in a therapeutic protocol, resulting in reversal of established inflammation.

Previously published studies, including the work on VE-202 by Atarashi et al (2013) in a similar Il10^{-/-} knockout mouse model of colitis, showed that introduction of a Live Biotherapeutic Product (LBP) resulted in an increase of FOXP3+ CD4+ T cells. This increase in FOXP3+ CD4+ T cells was defined as the key driver behind the inflammation control by these LBPs and part of homeostatic response to inflammation. Unexpectedly, in the current therapeutic intervention study with GUT-108 using ex-germ-free (sterile) Il10^{-/-} 129SvEv mice inoculated with human stool, a decrease was observed in FOXP3+ CD4+ T cells including FOXP3+RORγt+ CD4+ T cells (see FIGS. 18A and 18B) two weeks after the application of GUT-108 started. It has been previously reported that there is an increased number of FOXP3+ CD4+ T cells in inflamed mucosal tissues in patients with active ulcerative colitis and Crohn's disease, and that levels of FOXP3+ CD4+ T cells go down to detection limits in non-inflamed tissues (Yu et al, 2007; Ban et al, 2008). Thus, the unexpected decrease in FOXP3+ CD4+ T cells is indicative of superior control of inflammation by GUT-108 compared to other LBPs that have been tested in the same mouse model. This decrease in inflammation is further supported by the range of complementary data, including: the decrease of IFN-γ+, IL-17α+ and IFN-γ+ IL-17α+ synthesizing CD4+ T cells in the tissue of the colonic lamina propria; the decreased synthesis of pro-inflammatory IL-1b, IL-6IL-17α, IFNγ and TNFα; an improved blinded histology score; and decreased levels of Lipocalin-2 in the stool, a biomarker for inflammation, and increased levels of key therapeutic metabolites with anti-inflammatory activities (SCFA, indole and derivatives) or antagonistic activities (secondary bile acids) against pathogenic microorganisms.

### REFERENCES

Atarashi, K., T. Tanoue, K. Oshima, W. Suda, Y. Nagano, H. Nishikawa, S. Fukuda, T. Saito, S. Narushima, K. Hase, S. Kim, J. V. Fritz, P. Wilmes, S. Ueha, K. Matsushima, H. Ohno, B. Olle, S. Sakaguchi, T. Taniguchi, H. Morita, M. Hattori and K. Honda (2013). “Treg induction by a rationally selected mixture of Clostridia strains from the human microbiota.” Nature 500 (7461): 232-236.
Ban, A., M. Shioya, A. Nishida, T. Tsujikawa and Y. Fujiyama (2008). “Increased number of FoxP3+ CD4+ regulatory T cells in inflammatory bowel disease.” Molecular Medicine Reports 1: 647-650.
Bhattacharjee, S. and W. J. Lukiw (2013). “Alzheimer's disease and the microbiome.” Frontiers in Cellular Neuroscience 7: e153.
Boulangé, C. L., A. L. Neves, J. Chilloux, J. K. Nicholson and M.-E. Dumas (2016). “Impact of the gut microbiota on inflammation, obesity, and metabolic disease.” Genome Medicine 8:42.
Canani, R. B., M. Di Costanzo, G. Bedogni, A. Amoroso, L. Cosenza, C. Di Scala, V. Granata and R. Nocerino (2017). “Extensively hydrolyzed casein formula containing Lactobacillus rhamnosus GG reduces the occurrence of other allergic manifestations in children with cow's milk allergy: 3-year randomized controlled trial.” J Allergy Clin Immunol In press.
Canani, R. B., N. Sangwan, A. T. Stefka, R. Nocerino, L. Paparo, R. Aitoro, A. Calignano, A. A. Khan, J. A. Gilbert and C. R. Nagler (2016). “Lactobacillus rhamnosus GG-supplemented formula expands butyrate-producing bacterial strains in food allergic infants.” ISME Journal 10: 742-750.
De Souza, H. S. P. and C. Fiocchi (2016). “Immunopathogenesis of IBD: current state of the art.” Nature Rev. Gastroenterology Hepatology 13: 13-27.
Durand, G. A., P.-E. Fournier, D. Raoult and S. Edouard (2017). “‘Bittarella massiliensis’ gen. nov., sp. nov. isolated by culturomics from the gut of a healthy 28-year-old man.” New Microbe and New Infect. 16: 28-29
Eeckhaut, V., B. Flahou, C. Romero, S. Van DerHeyden, F. Pasmans, F. Haesebrouck, R. Ducatelle and F. Van Immerseel (2009). “The anaerobic butyrate-producing strain Butyricicoccus pullicaecorum decreases colonic inflammation and ulceration in a TNBS-induced colitis rat model.” In 5th Probiotics, Prebiotics and New Foods Congress, Rome, Italy. http://www.probiotics-prebiotics newfood.orepdf/5th_Probiotics_Prebiotics_Newfood.pdf.
Furusawa, Y., Y. Obata, S. Fukuda, T. A. Endo, G. Nakato, D. Takahashi, Y. Nakanishi, C. Uetake, K. Kato, T. Kato, M. Takahashi, N. N. Fukuda, S. Murakami, E. Miyauchi, S. Hino, K. Atarashi, S. Onawa, Y. Fujimura, T. Lockett, J. M. Clarke, D. L. Topping, M. Tomita, S. Hori, O. Ohara, T. Morita, H. Koseki, J. Kikuchi, K. Honda, K. Hase and H. Ohno (2013). “Commensal microbe-derived butyrate induces the differentiation of colonic regulatory T cells.” Nature 504: 446-450.
Gevers, D., S. Kugathasan, D. Knights, A. D. Kostic, R. Knight and R. J. Xavier (2017). “A Microbiome Foundation for the Study of Crohn's Disease.” Cell Host & Microbe 21: 301-304.
Halfvarson, J., C. J. Brislawn, R. Lamendella, Y. Vazquez-Baeza, W. A. Walters, L. M. Bramer, M. D'Amato, F. Bonfiglio, D. McDonald, A. Gonzalez, E. E. McClure, M. F. Dunklebarger, R. Knight and J. K. Jansson (2017). “Dynamics of the human gut microbiome in inflammatory bowel disease.” Nature Microbiol. 2: e17004.
Henry, C. S., M. DeJongh, A. A. Best, P. M. Frybarger, B. Linsay, R. L. Stevens (2010). “High-Throughput Generation, Optimization and Analysis of Genome-Scale Metabolic Models.” Nature Biotechnol. 28: 977-982.
Honda, K., K. Atarashi, K. Itoh and T. Tanoue (2015). Composition for inducing proliferation or accumulation of regulatory T cells. T. U. o. Tokyo. Japan. U.S. Pat. No. 9,415,079 B2.
Li, Q. and J.-M. Zhou (2016). “The microbiota-gut-brain axis and its potential therapeutic role in autism spectrum disorder” Neuroscience 324: 131-139.
Liu, B., S. L. Tonkonogy and R. B. Sartor (2011). Antigen-presenting cell production of IL-10 inhibits T-helper 1 and 17 cell responses and suppresses colitis in mice. Gastroenterology 141: 653-662.
Magnusson, M. K., S. F. Brynjolfsson, A. Dige, H. Uronen-Hansson, L. G. Börjesson, J. L. Bengtsson, S. Gudjonsson, L. Öhman, J. Agnholt, H. Sjövall, W. W. Agace, and M. J. Wick (2016). “Macrophage and Dendritic Cell Subsets in IBD: ALDH+ Cells Are Reduced in Colon Tissue of Patients with Ulcerative Colitis Regardless of Inflammation.” Mucosal Immunol. 9: 171-182.
Moos, W. H., D. V. Faller, D. N. Harpp, I. Kanara, J. Pernokas, W. R. Powers and K. Steliou (2016). “Microbiota and Neurological Disorders: A Gut Feeling.” BioResearch Open Access 5: 137-145.
Narushima, S., Y. Sugiura, K. Oshima, K. Atarashi, M. Hattori, M. Suematsu and K. Honda (2014). “Characterization of the 17 strains of regulatory T cell-inducing human-derived Clostridia.” Gut Microbes 5(3): 333-339.
Neavin, D. R., D. Liu, B. Ray, and R. M. Weinshilboum (2018). “The Role of the Aryl Hydrocarbon Receptor (AHR) in Immune and Inflammatory Diseases.” Int. J. Mol. Sci. 19: 3851-3866.
Pandiyan, P., X. P. Yang, S. S. Saravanamuthu, et al. (2012). “The role of IL-15 in activating STATS and fine-tuning IL-17A production in CD4 T lymphocytes.” J. Immunol. 189: 4237-4246
Paun, A. and J. S. Danska (2016). “Modulation of type 1 and type 2 diabetes risk by the intestinal microbiome.” Pediatr Diabetes 17(7): 469-477.
Ramasundara, M., S. T. Leach, D. A. Lemberg, and A. S. Day (2009). “Defensins and inflammation: The role of defensins in inflammatory bowel disease.” J. Gastroenterology Hepatology 24: 202-208.
Rogers, G. B., D. J. Keating, R. L. Young, M.-L. Wong, J. J Licinio and S. Wesselingh (2016). “From gut dysbiosis to altered brain function and mental illness: mechanisms and pathways.” Molecular Psychiatry 21: 738-748.
Sartor, B. R. and G D. Wu (2017). “Roles for intestinal bacteria, viruses, and fungi in pathogenesis of inflammatory bowel diseases and therapeutic approaches”. Gastroenterology 152 (2): 327-339.e4.
Schwabe, R. F. and C. Jobin (2013). “The microbiome and cancer.” Nature Reviews Cancer 13: 800-812.
Seekatz, A. M. R., K. Santhosh, K. Young, V. B. (2016). “Dynamics of the fecal microbiome in patients with recurrent and nonrecurrent Clostridium difficile infection.” Genome Medicine 8:47.
Sellon, R. K., S. Tonkonogy, M. Schultz, L. A. Dieleman, W. Grenther, E. Balish, D. A. Rennick, and R. B. Sartor (1998). “Resident enteric bacteria are necessary for development of spontaneous colitis and immune system activation in interleukin-10-deficient mice.” Infection Immunity 66: 5224-5231.
Sokol, H., B. Pigneur, L. Watterlot, O. Lakhdari, L. G. Bermúdez-Humarán, J. J. Gratadoux, S. Blugeon, C. Bridonneau, J. P. Furet, G. Corthier, C. Grangette, N. Vasquez, P. Pochart, G. Trugnan, G. Thomas, H. M. Blottière, J. Doré, P. Marteau, P. Seksik and P. Langella (2008). “Faecalibacterium prausnitzii is an anti-inflammatory commensal bacterium identified by gut microbiota analysis of Crohn disease patients.” Proc Natl Acad Sci USA 105(43): 16731-16736.
Sun, J. and I. Kato (2016). “Gut microbiota, inflammation and colorectal cancer.” Genes & Diseases 3: 130-143.
Van Immerseel, F., R. Ducatelle, M. De Vos, N. Boon, T. Van De Wiele, K. Verbeke, P. Rutgeerts, B. Sas, P. Louis and H. J. Flint (2010). “Butyric acid-producing anaerobic bacteria as a novel probiotic treatment approach for inflammatory bowel disease.” Journal of Medical Microbiology 59: 142-143.
Vila, A. V., F. Imhann, V. Collij, S. A. Jankipersadsing, T. Gurry, Z. Mujagic, A. Kurilshikov, M. J. Bonder, X. Jiang, E. F. Tigchelaar, J. Dekens, V. Peters, M. D. Voskuil, M. C. Visschedijk, H. M. van Dullemen, D. Keszthelyi, M. A. Swertz, L. Franke, R. Alberts, E. A. M. Festen, G. Dijkstra, A. A. M. Masclee, M. H. Hofker, R. J. Xavier, E. J. Alm, J. Fu, C. Wijmenga, D. M. A. E. Jonkers, A. Zhernakova, R. K. Weersma (2018). “Gut Microbiota Composition and Functional Changes in Inflammatory Bowel Disease and Irritable Bowel Syndrome.” Sci. Transl. Med. 10, eaap8914.
Vuong, H. E. H., and Hsiao, E. Y. (2017). “Emerging Roles for the Gut Microbiome in Autism Spectrum Disorder.” Biological Psychiatry 5: 411-423.
Yu, Q. T., M. Saruta, A. Avanesyan, P. R. Fleshner, A. H. Banham, and K. A. Papadakis (2007). “Expression and Functional Characterization of FOXP3+CD4+ Regulatory T Cells in Ulcerative Colitis”. Inflamm. Bowel Dis. 13: 191-199.
Zheng, X., Y. Qiu, W. Zhong, S. Baxter, M. Su, Q. Li, G. Xie, B. M. Ore, S. Qiao, M. D. Spencer, S. H. Zeisel, Z. Zhou, A. Zhao, W. Jia (2013). “A Targeted Metabolomic Protocol for Short-Chain Fatty Acids and Branched-Chain Amino Acids”. Metabolomics 9: 818-827.

1-24. (canceled)

25. A composition for use in a method of benefiting the health of an animal or a human comprising a biologically pure culture of two or more of a bacterium having 97% sequence identity to the 16S rRNA gene sequence of: Akkermansia species—like strain GGCC_0220 (SEQ ID NO: 1), Bacteroides uniformis—like strain GGCC_0301 (SEQ ID NO: 2), Bacteroides xylanisolvens—like strain GGCC_0124 (SEQ ID NO: 3), Barnesiella species—like strain GGCC_0306 (SEQ ID NO: 4), Bitterella massiliensis—like strain GGCC_0305 (SEQ ID NO: 5), Clostridium butyricum—like strain GGCC_0151 (SEQ ID NO: 6), Clostridium scindens—like strain GGCC_0168 (SEQ ID NO: 7), Clostridium symbiosum—like strain GGCC_0272 (SEQ ID NO: 8), Eubacterium callanderi—like strain GGCC_0197 (SEQ ID NO: 9), Extibacter species—like strain GGCC_0201 (SEQ ID NO: 10) and Intestinimonas butyriciproducens—like strain GGCC 0179 (SEQ ID NO: 11).

26. The composition of claim 25, comprising a biologically pure culture of each of: Akkermansia species—like strain GGCC_0220, Bacteroides uniformis—like strain GGCC_0301, Bacteroides xylanisolvens—like strain GGCC_0124, Barnesiella species—like strain GGCC_0306, Bitterella massiliensis—like strain GGCC_0305, Clostridium butyricum—like strain GGCC_0151, Clostridium scindens—like strain GGCC_0168, Clostridium symbiosum—like strain GGCC_0272, Eubacterium callanderi—like strain GGCC_0197, Extibacter species—like strain GGCC_0201 and Intestinimonas butyriciproducens—like strain GGCC 0179.

27. The composition of claim 25, comprising a biologically pure culture of two or more of: Akkermansia species—like strain GGCC_0220, Bacteroides uniformis—like strain GGCC_0301, Bacteroides xylanisolvens—like strain GGCC 0124, Barnesiella species—like strain GGCC_0306, Bitterella massiliensis—like strain GGCC_0305, Clostridium butyricum—like strain GGCC 0151, Clostridium scindens—like strain GGCC 0168, Clostridium symbiosum—like strain GGCC 0272, Eubacterium callanderi—like strain GGCC 0197, Extibacter species—like strain GGCC 0201 and Intestinimonas butyriciproducens—like strain GGCC 0179.

28. The composition of claim 25, wherein:
a) the biologically pure culture of the bacterium having 97% sequence identity to the 16S rRNA gene sequence of Akkermansia species—like strain GGCC 0220 (SEQ ID NO: 1) further comprises genetic material encoding for synthesis of propionate, synthesis of 4-amino-butyrate (gamma-aminobutyric acid; GABA), and synthesis of indole or indole-containing secondary metabolites;
b) the biologically pure culture of the bacterium having 97% sequence identity to the 16S rRNA gene sequence of Bacteroides uniformis—like strain GGCC_0301 (SEQ ID NO: 2) further comprises genetic material encoding for synthesis of propionate, synthesis of GABA, and synthesis of indole or indole-containing secondary metabolites;
c) the biologically pure culture of the bacterium having 97% sequence identity to the 16S rRNA gene sequence of Bacteroides xylanisolvens—like strain GGCC 0124 (SEQ ID NO: 3) further comprises genetic material encoding for synthesis of propionate, synthesis of GABA, and synthesis of indole or indole-containing secondary metabolites;
d) the biologically pure culture of the bacterium having 99% sequence identity to the 16S rRNA gene sequence of Barnesiella species—like strain GGCC_0306 (SEQ ID NO: 4) further comprises genetic material encoding for synthesis of propionate and synthesis of GAB A;
e) the biologically pure culture of the bacterium having 97% sequence identity to the 16S rRNA gene sequence of Bitterella massiliensis—like strain GGCC_0305 (SEQ ID NO: 5) further comprises genetic material encoding for 3α-hydroxy steroid dehydrogenase and 3β-hydroxy steroid dehydrogenase;
f) the biologically pure culture of the bacterium having 97% sequence identity to the 16S rRNA gene sequence of Clostridium butyricum—like strain GGCC 0151 (SEQ ID NO: 6) further comprises genetic material encoding for synthesis of butyrate;
g) the biologically pure culture of the bacterium having 97% sequence identity to the 16S rRNA gene sequence of Clostridium scindens—like strain GGCC 0168 (SEQ ID NO: 7) further comprises genetic material encoding for bile acid 7-alpha-dehydratase activity;
h) the biologically pure culture of the bacterium having 97% sequence identity to the 16S rRNA gene sequence of Clostridium symbiosum—like strain GGCC 0272 (SEQ ID NO: 8) further comprises genetic material encoding for synthesis of butyrate;
i) the biologically pure culture of the bacterium having 97% sequence identity to the 16S rRNA gene sequence of Eubacterium callanderi—like strain GGCC 0197 (SEQ ID NO: 9) further comprises genetic material encoding for synthesis of butyrate, synthesis of GABA, and bile acid 7-alpha-dehydratase activity;
j) the biologically pure culture of the bacterium having 97% sequence identity to the 16S rRNA gene sequence of Extibacter species—like strain GGCC 0201 (SEQ ID NO: 10) further comprises genetic material encoding for bile acid 7-alpha-dehydratase activity; and
k) the biologically pure culture of the bacterium having 97% sequence identity to the 16S rRNA gene sequence of Intestinimonas butyriciproducens—like strain GGCC 0179 (SEQ ID NO: 11) further comprises genetic material encoding for synthesis of butyrate.

29. The composition of claim 25, wherein the composition comprises three, four, five, six, seven, eight, nine, ten, or eleven of the biologically pure cultures.

30. The composition of claim 25, comprising a biologically pure culture of each of: Akkermansia species—like strain GGCC 0220, Bacteroides uniformis—like strain GGCC 0301, Bacteroides xylanisolvens—like strain GGCC 0124, Clostridium butyricum—like strain GGCC_0151, Clostridium scindens—like strain GGCC_0168, Clostridium symbiosum—like strain GGCC_0272, Eubacterium callanderi—like strain GGCC_0197, Extibacter species—like strain GGCC_0201 and Intestinimonas butyriciproducens—like strain GGCC_0179.

31. The composition of claim 25, comprising a biologically pure culture of each of a bacterium having 97% sequence identity to the 16S rRNA gene sequence of: Akkermansia species—like strain GGCC_0220 (SEQ ID NO: 1), Bacteroides uniformis—like strain GGCC_0301 (SEQ ID NO: 2), Bacteroides xylanisolvens—like strain GGCC 0124 (SEQ ID NO: 3), Clostridium butyricum—like strain GGCC_0151 (SEQ ID NO: 6), Clostridium scindens—like strain GGCC_0168 (SEQ ID NO: 7), Clostridium symbiosum—like strain GGCC_0272 (SEQ ID NO: 8), Eubacterium callanderi—like strain GGCC_0197 (SEQ ID NO: 9), Extibacter species—like strain GGCC_0201 (SEQ ID NO: 10) and Intestinimonas butyriciproducens—like strain GGCC_0179 (SEQ ID NO: 11).

32. The composition of claim 31, wherein:
a) the biologically pure culture of the bacterium having 97% sequence identity to the 16S rRNA gene sequence of Akkermansia species—like strain GGCC_0220 (SEQ ID NO: 1) further comprises genetic material encoding for synthesis of propionate, synthesis of 4-amino-butyrate (gamma-aminobutyric acid; GABA), and synthesis of indole or indole-containing secondary metabolites;
b) the biologically pure culture of the bacterium having 97% sequence identity to the 16S rRNA gene sequence of Bacteroides uniformis—like strain GGCC_0301 (SEQ ID NO: 2) further comprises genetic material encoding for synthesis of propionate, synthesis of GABA, synthesis of indole or indole-containing secondary metabolites, and deconjugation and conversion of bile salts into secondary bile acids;
c) the biologically pure culture of the bacterium having 97% sequence identity to the 16S rRNA gene sequence of Bacteroides xylanisolvens—like strain GGCC 0124 (SEQ ID NO: 3) further comprises genetic material encoding for synthesis of propionate, synthesis of GABA, and synthesis of indole or indole-containing secondary metabolites;
d) the biologically pure culture of the bacterium 97% sequence identity to the 16S rRNA gene sequence of Clostridium butyricum—like strain GGCC 0151 (SEQ ID NO: 6) further comprises genetic material encoding for synthesis of butyrate;
e) the biologically pure culture of the bacterium having 97% sequence identity to the 16S rRNA gene sequence of Clostridium scindens—like strain GGCC 0168 (SEQ ID NO: 7) further comprises genetic material encoding for bile acid 7-alpha-dehydratase activity;
f) the biologically pure culture of the bacterium having 97% sequence identity to the 16S rRNA gene sequence of Clostridium symbiosum—like strain GGCC 0272 (SEQ ID NO: 8) further comprises genetic material encoding for synthesis of butyrate;
g) the biologically pure culture of the bacterium having 97% sequence identity to the 16S rRNA gene sequence of Eubacterium callanderi—like strain GGCC 0197 (SEQ ID NO: 9) further comprises genetic material encoding for synthesis of butyrate, synthesis of GABA, and bile acid 7-alpha-dehydratase activity;
h) the biologically pure culture of the bacterium having 97% sequence identity to the 16S rRNA gene sequence of Extibacter species—like strain GGCC 0201 (SEQ ID NO: 10), further comprises genetic material encoding for bile acid 7-alpha-dehydratase activity; and
i) the biologically pure culture of the bacterium having 97% sequence identity to the 16S rRNA gene sequence of Intestinimonas butyriciproducens—like strain GGCC 0179 (SEQ ID NO: 11) further comprises genetic material encoding for synthesis of butyrate.

33. The composition of claim 25, wherein the composition is formulated for delivery to the intestine.

34. The composition of claim 25, further comprising one or more pharmaceutically acceptable carriers.

35. The composition of claim 25, wherein the biologically pure cultures of bacterial strains are in the form of spores or vegetative cells or a combination thereof.

36. The composition of claim 25, wherein the biologically pure cultures of bacterial strains are in the form of freeze-dried spores or vegetative cells or a combination thereof.

37. The composition of claim 25, wherein the biologically pure cultures of bacterial strains comprise complementary auxotrophies to provide metabolic interdependency.

38. A method of treating an inflammatory disorder in a subject, comprising:
administering to the subject a composition of claim 25,
wherein the administering results in a decrease in inflammation.

39. A method of improving the health of the microbiome of a subject, comprising:
administering to the subject a composition of claim 25;
wherein the administering results in an improvement in the health of the subject's microbiome.

40. The method of claim 38, wherein the subject is a human and the administering to the subject is on a daily basis.

41. The method of claim 38, wherein the inflammatory disorder comprises Inflammatory Bowel Disease (IBD), ulcerative colitis, Crohn's disease, Ankylosing Spondylitis, Plaque Psoriasis, Psoriatic Arthritis or Pouchitis.

42. The method of claim 38, wherein the inflammatory disorder comprises Alzheimer's and Non-Alzheimer's disease related dementia, Parkinson's disease, multiple sclerosis, Type-2 diabetes, obesity, hepatic steatosis, Primary Sclerosing Cholangitis as comorbidity for IBD, or aging related cardiovascular or neurologic conditions.

43. The method of claim 38, wherein the inflammatory disorder comprises infection or overpopulation with one or a combination of pathogenic species of the Enterobacteriaceae or pathogenic species of Clostridium.

44. The method of claim 38, comprising administering the composition in combination with one or both a corticosteroid and one or a combination of the drugs listed in Table 3.
